(19) United States
(12) Patent Application Publication
(10) Pub. No.: US 2024/0127961 A1
(43) Pub. Date: Apr. 18, 2024

(54) SYSTEM AND METHOD FOR ADJUSTING HYPOXIA-INDUCIBLE FACTOR STABILIZER TREATMENT BASED ON ANEMIA MODELING

(71) Applicants: Fresenius Medical Care Holdings, Inc., Waltham, MA (US); Fresenius Medical Care Deutschland GmbH, Bad Homburg (DE)

(72) Inventors: Doris Fuertinger, Long Island City, NY (US); David Joerg, Bad Homburg (DE); Gudrun Schappacher-Tilp, Waltham, MA (US); Peter Kotanko, New York, NY (US)

(21) Appl. No.: 17/960,305

(22) Filed: Oct. 5, 2022

(51) Int. Cl. G16H 50/50; G16H 10/20; G16H 20/10; G16H 20/40; G16H 50/70

(52) U.S. Cl. CPC G16H 50/50; G16H 10/20; G16H 20/10; G16H 20/40; G16H 50/70

(57) ABSTRACT

A method for determining a next hypoxia-inducible factor prolyl hydroxylase inhibitor (HIF-PHI) dosage for a first patient using a patient HIF-PHI model is provided. The method includes obtaining population patient data indicating HIF-PHI dosages and hemoglobin measurements for the patients. Then, virtual patient avatars are generated based on the population patient data. Each of the virtual patient avatars indicates a set of personalized model parameters for a HIF-PHI model. A plurality of HIF-PHI models are determined for the virtual patient avatars. Using the HIF-PHI models, one or more HIF-PHI treatment schemes for administering the HIF-PHI dosages is determined. Subsequently, the HIF-PHI treatment schemes along with a hematocrit and/or hemoglobin concentration for a patient are used to determine a next HIF-PHI dosage for the patient, and the next HIF-PHI dosage is administered for the patient.

### BACKGROUND

Red blood cells (erythrocytes) are essential for the transport of oxygen through the body. An understanding of the regulation of red blood cell production, called erythropoiesis, is important for the treatment of patients in a variety of clinical situations. Patients may be prescribed hypoxia-inducible factor prolyl hydroxylase (HIF-PH) inhibitors (HIF-PHIs) or HIF stabilizers, which are members of a class of drugs that act by inhibiting HIF-PH that are responsible to break down the HIF under conditions of normal oxygen concentrations. Such patients include, but are not limited to chronic kidney disease patients, patients scheduled for surgery, dialysis patients, and/or other patients. However, the dose and frequency of administration of the HIF stabilizer treatments are often determined based on the prior experience of the physician and guidance such as manufacturer guidance and/or professional guidance, because predictive models of HIF-PHIs are not readily available.

### SUMMARY

This summary is provided to introduce certain exemplary embodiments that are further described below. This summary is not intended to be an identification of key features or essential features of the present disclosure.

In an embodiment, a method of determining a next hypoxia-inducible factor prolyl hydroxylase inhibitor (HIF-PHI) dosage for a first patient using a patient HIF-PHI model is provided. The method comprises: obtaining population patient data associated with a plurality of patients, wherein the population patient data indicates previous HIF-PHI dosages and hemoglobin measurements for the plurality of patients; generating a plurality of virtual patient avatars based on the population patient data, wherein each of the plurality of virtual patient avatars indicates a set of personalized model parameters; determining a plurality of HIF-PHI models for the plurality of virtual patient avatars based on the set of personalized model parameters; determining one or more HIF-PHI treatment schemes for administration of HIF-PHI dosages based on the plurality of HIF-PHI models; and administering the next HIF-PHI dosage for the first patient, of the plurality of patients, based on using the one or more determined HIF-PHI treatment schemes and a hematocrit and/or hemoglobin concentration for the first patient.

In some instances, the method further comprises: obtaining individualized patient data for a second patient, of the plurality of patients, wherein the individualized patient data indicates a previous HIF-PHI dosage and a hemoglobin measurement for the second patient; determining a patient HIF-PHI model for the second patient based on the previous HIF-PHI dosage, the hemoglobin measurement, and a mathematical model for hydroxylase inhibitor (HIF) stabilizer treatment, wherein the HIF-PHI model indicates a set of individualized model parameters for the second patient; based on the second patient's hemoglobin measurement being outside of a patient threshold, employing the patient HIF-PHI model to determine a next HIF-PHI dosage for the second patient; and administering the next HIF-PHI dosage to the second patient to adjust the hemoglobin concentration to be within the patient threshold.

In some examples, administering the next HIF-PHI dosage comprises: causing display of the next HIF-PHI dosage on a display device.

In some variations, the method further comprises: obtaining a mathematical model for hydroxylase inhibitor (HIF) stabilizer treatment; wherein determining the plurality of HIF-PHI models for the plurality of virtual patient avatars comprises generating the plurality of HIF-PHI models by inserting the set of personalized model parameters into the mathematical model.

In some instances, each of the one or more HIF-PHI treatment schemes indicates a decision tree comprising a plurality of branches indicating different HIF-PHI dosages based on hemoglobin concentrations.

In some examples, determining the one or more HIF-PHI treatment schemes comprises: generating a plurality of HIF-PHI treatment schemes for performing HIF stabilizer treatment; simulating a plurality of virtual trials using the plurality of HIF-PHI treatment schemes and the plurality of HIF-PHI models for the plurality of virtual patient avatars; and determining the one or more HIF-PHI treatment schemes based on simulating the plurality of virtual trials.

In some variations, determining the one or more HIF-PHI treatment schemes is based on a number of patients, from the plurality of patients, within a target hemoglobin threshold and an amount of HIF-PHI medication administered to a plurality of simulated patients within the plurality of virtual trials.

In some instances, administering the next HIF-PHI dosage for the first patient comprises causing display of the next HIF-PHI dosage for the first patient on a display device.

In some examples, the set of personalized model parameters comprises a HIF-PHI bioavailability parameter, a red blood cell (RBC) lifespan parameter, and a hemoglobin set point parameter, and wherein generating the plurality of virtual patient avatars comprises determining the HIF-PHI bioavailability parameter, the red blood cell (RBC) lifespan parameter, and the hemoglobin set point parameter for each of the plurality of virtual patient avatars.

In some variations, the set of personalized model parameters further comprises a basal erythropoietin (EPO) synthesis rate parameter, a HIF signal threshold parameter, and a hepcidin decay rate parameter, and wherein generating the plurality of virtual patient avatars further comprises determining the basal EPO synthesis rate parameter, the HIF signal threshold parameter, and the hepcidin decay rate parameter for each of the plurality of virtual patient avatars.

In some instances, determining the plurality of HIF-PHI models for the plurality of virtual patient avatars is further based on the following equation:

$$\frac{dh}{dt} = {β}_{h} ( θ ) [ {φ}_{{a}_{h} , {b}_{h} , {q}_{h , 0}} ( {k}_{h} [ {c}_{hgb} - {c}_{hgb}^{*} ] ) + {E}_{s} {ϕ}^{+} ( {[ \frac{s}{\overline{s}} ]}^{{m}_{h}} ) ] - {k}_{h} h ,$$

where dh/dt at indicates a rate of change of HIF signaling activity, βₕ(θ) indicates a formation rate of HIF complexes, φₐ_{ₕ}_{,b}_{ₕ}_{,q}_{_{h,0}} indicates a hypoxic upregulation of a HIF signal in response to decreases in blood hemoglobin concentration, kₕ indicates a decay rate of the HIF signal, c_{hgb} indicates a blood hemoglobin concentration measured by kidneys, c*_{hgb} indicates a physiological hemoglobin set point, Eₛ indicates a HIF-PHI responsiveness,

$${ϕ}^{+} ( {[ \frac{s}{\overline{s}} ]}^{{m}_{h}} )$$

indicates upregulation of the HIF signal in response to HIF-PHI administrations, and h indicates the HIF signal.

In some examples, determining the plurality of HIF-PHI models for the plurality of virtual patient avatars is further based on the following equation:

$$\frac{de}{dt} = {β}_{e} ( θ ) + {β}_{e} ( θ ) {φ}_{{a}_{e} , {b}_{e} , {q}_{e , 0}} ( {k}_{e} h ) - {k}_{e} e ,$$

where de/dt indicates a EPO concentration rate of change, βₑ(θ) indicates basal erythropoietin (EPO) synthesis under steady-state conditions, βₑ(θ)φₐ_{ₑ}_{,b}_{ₑ}_{,q}_{_{e,0}}(kₑh) indicates additional EPO synthesis due to activation by a HIF signal, and kₑe indicates EPO decay with a decay rate kₑ.

In some variations, determining the plurality of HIF-PHI models for the plurality of virtual patient avatars is further based on the following equation:

$$\frac{{dn}_{ery}}{dt} = {2}^{{d}_{pre}} {J}_{pre \to ery} - {K}_{ery} - {L}_{bleeding} + {L}_{donation} , where \frac{{dn}_{ery}}{dt}$$

indicates a rate of change of the total amount of red blood cells, 2^{d}^{ₚᵣₑ} J_{pre→ery} indicates a differentiation flux from a precursors population to an erythrocytes population, K_{ery} indicates cell fluxes due to apoptosis, L_{bleeding} indicates cell fluxes due to bleeding events, and L_{donation} indicates cell fluxes due to blood donation events.

In another embodiment, a method of adjusting a patient's hematocrit and/or hemoglobin concentration using a patient hypoxia-inducible factor prolyl hydroxylase inhibitor (HIF-PHI) model is provided. The method comprises: obtaining individualized patient data for the patient, wherein the individualized patient data indicates a previous HIF-PHI dosage and a hemoglobin measurement for the patient; determining a patient HIF-PHI model for the patient based on the previous HIF-PHI dosage, the hemoglobin measurement, and a mathematical model for hydroxylase inhibitor (HIF) stabilizer treatment, wherein the HIF-PHI model indicates a set of individualized model parameters for the patient; based on the patient's hematocrit and/or hemoglobin concentration being outside of a patient threshold, employing the patient HIF-PHI model to determine a next HIF-PHI dosage for the patient; and administering the next HIF-PHI dosage to the patient to adjust the hematocrit and/or the hemoglobin concentration to be within the patient threshold.

In some instances, administering the next HIF-PHI dosage comprises: causing display of the next HIF-PHI dosage on a display device.

In some examples, the set of individualized model parameters comprises a HIF-PHI bioavailability parameter, a red blood cell (RBC) lifespan parameter, and a hemoglobin set point parameter, and wherein determining the next HIF-PHI dosage for the patient is based on using the HIF-PHI bioavailability parameter, the red blood cell (RBC) lifespan parameter, and the hemoglobin set point parameter.

In some variations, the set of individualized model parameters further comprises a basal erythropoietin (EPO) synthesis rate parameter, a HIF signal threshold parameter, and a hepcidin decay rate parameter, and wherein determining the next HIF-PHI dosage for the patient is further based on the basal EPO synthesis rate parameter, the HIF signal threshold parameter, and the hepcidin decay rate parameter.

In some instances, employing the patient HIF-PHI model to determine the next HIF-PHI dosage for the patient comprises: inputting a plurality of next HIF-PHI dosages into the HIF-PHI model to determine a plurality of outputs indicating expected hematocrit or hemoglobin concentrations; and selecting the next HIF-PHI dosage from the plurality of next HIF-PHI dosages based on an output, of the plurality of outputs, being within the patient threshold.

In some examples, employing the patient HIF-PHI model to determine the next HIF-PHI dosage for the patient comprises: inputting a plurality of next HIF-PHI dosages into the HIF-PHI model to determine a plurality of outputs indicating expected hematocrit or hemoglobin concentrations; determining a subset of next HIF-PHI dosages, of the plurality of next HIF-PHI dosages, based on one or more outputs, of the plurality of outputs, associated with the subset of next HIF-PHI dosages being within the patient threshold; and selecting the next HIF-PHI dosage from the subset of next HIF-PHI dosages based on the next HIF-PHI dosage being a lowest amount of HIF-PHI dosage within the subset of next HIF-PHI dosages.

In another embodiment, a computing device is provided. The computing device comprises: a display configured to display information associated with a patient hypoxia-inducible factor prolyl hydroxylase inhibitor (HIF-PHI) model; one or more processors; and a non-transitory computer-readable medium having processor-executable instructions stored thereon, wherein the processor-executable instructions, when executed by the one or more processors, facilitate: obtaining population patient data associated with a plurality of patients, wherein the population patient data indicates previous HIF-PHI dosages and hemoglobin measurements for the plurality of patients; generating a plurality of virtual patient avatars based on the population patient data, wherein each of the plurality of virtual patient avatars indicates a set of personalized model parameters; determining a plurality of HIF-PHI models for the plurality of virtual patient avatars based on the set of personalized model parameters; determining one or more HIF-PHI treatment schemes for administration of HIF-PHI dosages based on the plurality of HIF-PHI models; and displaying the next HIF-PHI dosage for the first patient, of the plurality of patients, on the display based on using the one or more determined HIF-PHI treatment schemes and a hematocrit and/or hemoglobin concentration for the first patient.

Further features and aspects are described in additional detail below with reference to the figures.

### BRIEF DESCRIPTION OF THE DRAWINGS

FIG. 1 is a schematic diagram of an exemplary medical treatment system according to one or more examples of the present application.

FIG. 2 is a simplified block diagram depicting an exemplary computing environment in accordance with one or more examples of the present application according to one or more examples of the present application.

FIG. 3 is a simplified block diagram of one or more devices or systems within the exemplary environment of FIG. 2 according to one or more examples of the present application.

FIG. 4 is a flowchart of an exemplary process for generating HIF-PHI models according to one or more examples of the present application.

FIG. 5A is a flowchart of an exemplary process for determining a next HIF-PHI dosage for a patient based on using a treatment scheme according to one or more examples of the present application.

FIG. 5B is an exemplary treatment scheme according to one or more examples of the present application.

FIG. 6 is a flowchart of an exemplary process for adjusting a patient's hematocrit and/or hemoglobin concentration using a HIF-PHI model according to one or more examples of the present application.

FIG. 7 is a block diagram of the exemplary mathematical model for the HIF stabilizer treatment according to one or more examples of the present application.

FIGS. 8A-8D are block diagrams describing blocks within the exemplary mathematical model of FIG. 7 according to one or more examples of the present application.

FIG. 9 is a data flowchart of an exemplary process for obtaining and using the population patient data according to one or more examples of the present application.

FIGS. 10A-10L show graphical representations for modeling HIF-PHI administrations according to one or more examples of the present application.

FIGS. 11A-11L show graphical representations for modeling HIF-PHI administrations according to one or more examples of the present application.

FIGS. 12A-12L show graphical representations for modeling HIF-PHI administrations according to one or more examples of the present application.

FIGS. 13A-13L show graphical representations for modeling HIF-PHI administrations according to one or more examples of the present application.

FIGS. 14A-14L show graphical representations for modeling HIF-PHI administrations according to one or more examples of the present application.

FIGS. 15A-15L show graphical representations for modeling HIF-PHI administrations according to one or more examples of the present application.

FIGS. 16A-16L show graphical representations for modeling HIF-PHI administrations according to one or more examples of the present application.

FIGS. 17A-17L show graphical representations for modeling HIF-PHI administrations according to one or more examples of the present application.

### DETAILED DESCRIPTION

Exemplary embodiments of the present application provide for generating HIF-PHI models for a plurality of patients and using the HIF-PHI models to administer HIF-PHI dosages to one or more patients to adjust the patients' hematocrit and/or hemoglobin concentrations. Examples of modeling erythropoiesis (including using iron homeostasis) with a selected erythropoiesis stimulating agent (ESA) administration regimen, predicting a patient's hematocrit and/or hemoglobin concentration with the initially selected ESA administration regimen, employing the model to determine one or more different ESA administration regimens such that the model predicts the patient's hematocrit and/or hemoglobin concentrations are within the desired range, and administering the ESA to the patient with the determined ESA administration regimen are described in U.S. Pat. No. 10,319,478, titled “SYSTEM AND METHOD OF MODELING ERYTHROPOIESIS AND ITS MANAGEMENT,” and U.S. Pat. No. 9,679,111, titled “SYSTEM AND METHOD OF MODELING ERYTHROPOIESIS INCLUDING IRON HOMEOSTATIS”, which are incorporated by reference in their entirety herein. However, while modeling erythropoiesis with ESA administration regimens have been described previously, HIF-PHIs are a new type of drug or medication that may be different from ESAs. For instance, HIF-PHIs may stimulate the HIF signaling pathway, which also mediates the body's endogenous response to hypoxic conditions, leading to enhanced production of endogenous erythropoietin (EPO). In contrast, ESAs directly mimic the action of EPOs, without directly targeting the HIF pathway. Furthermore, the time interval between consecutive ESA administrations is typically weeks or months apart whereas HIF-PHIs are typically administered multiple times a week. Accordingly, as will be explained in further detail below, the present disclosure describes a system and method for generating HIF-PHI models and using the HIF-PHI models to determine/adjust HIF-PHI dosages for one or more patients.

Among other advantages, the present disclosure uses HIF-PHI models to provide more accurate HIF-PHI dosages for patients undergoing HIF-PHI stabilizer treatment. For instance, because every patient is different, especially when reacting to different types of medications, the HIF-PHI models are used to predict the patient's bodily reactions to HIF-PHI dosages, which in turn is capable of determining a more accurate HIF-PHI dosage that causes the patient to better reach a desired hematocrit and/or hemoglobin concentration range while also alleviating insufficient erythropoiesis and preventing excessively high HIF-PHI dose levels that raise the patient's blood pressure and increase the patient's risk of stroke and cardiovascular disease. Additionally, and/or alternatively, the HIF-PHI dosages from the HIF-PHI models may lead to accelerated recovery of a patient's hemoglobin levels from events such as when the patient encounters bleeding episodes.

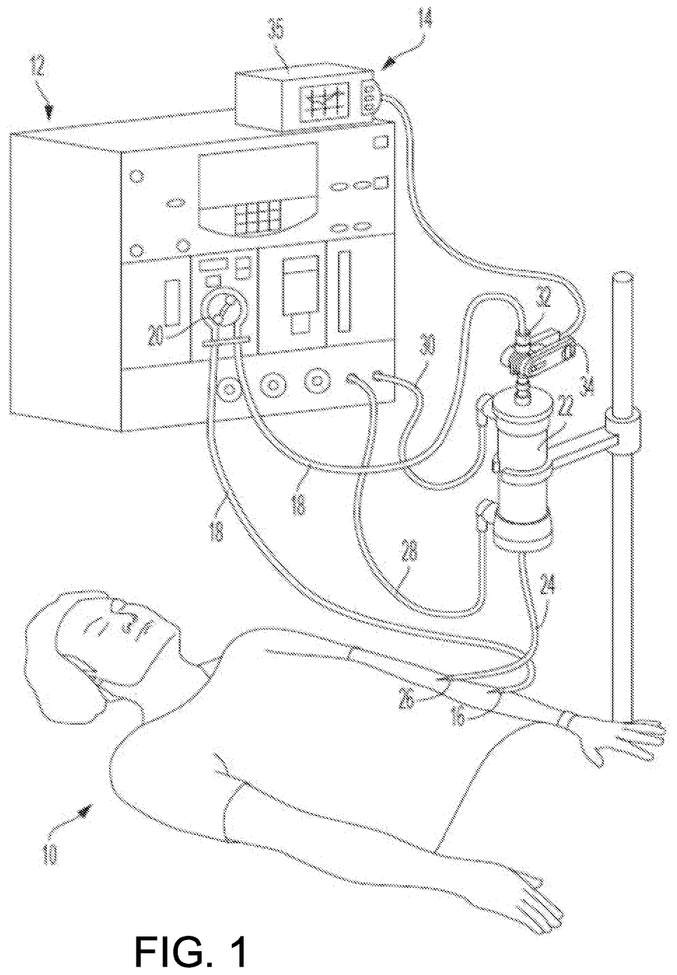

FIG. 1 is a schematic diagram of an exemplary medical system (e.g., a dialysis system) according to one or more examples of the present application. By way of example, the medical system shown in FIG. 1 is a hemodialysis system; however, other medical systems are contemplated. The hemodialysis system may be used to measure/determine hematocrit (HCT), hemoglobin (HGB), and/or absolute blood volumes (ABV) of a patient 10. For example, FIG. 1 depicts a patient 10 undergoing hemodialysis treatment using a hemodialysis machine 12. The hemodialysis system further includes an optical blood monitoring system 14.

An inlet needle or catheter 16 is inserted into an access site of the patient 10, such as in the arm, and is connected to extracorporeal tubing 18 that leads to a peristaltic pump 20 and to a dialyzer 22 (or blood filter). The dialyzer 22 removes toxins and excess fluid from the patient's blood. The dialyzed blood is returned from the dialyzer 22 through extracorporeal tubing 24 and return needle or catheter 26. In some parts of the world, the extracorporeal blood flow may additionally receive a heparin drip to prevent clotting. The excess fluids and toxins are removed by clean dialysate liquid which is supplied to the dialyzer 22 via tube 28, and waste liquid is removed for disposal via tube 30. A typical hemodialysis treatment session takes about 3 to 5 hours in the United States.

The optical blood monitoring system 14 includes a display device 35 and a sensor device 34. The sensor device 34 may, for example, be a sensor clip assembly that is clipped to a blood chamber 32, wherein the blood chamber 32 is disposed in the extracorporeal blood circuit. A controller (e.g., processor) of the optical blood monitoring system 14 may be implemented in the display device 35 or in the sensor clip assembly 34, or both the display device 35 and the sensor clip assembly 34 may include a respective controller for carrying out respective operations associated with the medical system.

The blood chamber 32 may be disposed in line with the extracorporeal tubing 18 upstream of the dialyzer 22. Blood from the peristaltic pump 20 flows through the tubing 18 into the blood chamber 32. The sensor device 34 includes emitters that emit light at certain wavelengths and detectors for receiving the emitted light after it has passed through the blood chamber 32. For example, the emitters may include LED emitters that emit light at approximately 810 nm, which is isobestic for red blood cells, at approximately 1300 nm, which is isobestic for water, and at approximately 660 nm, which is sensitive for oxygenated hemoglobin, and the detectors may include a silicon photodetector for detecting light at the approximately 660 and 810 nm wavelengths, and an indium gallium arsenide photodetector for detecting light at the approximately 1300 nm wavelength. The blood chamber 32 includes lenses or viewing windows that allows the light to pass through the blood chamber 32 and the blood flowing therein.

An example of an optical blood monitoring system having a sensor clip assembly configured to measure hematocrit and oxygen saturation of extracorporeal blood flowing through a blood chamber is described in U.S. Pat. No. 9,801,993, titled “SENSOR CLIP ASSEMBLY FOR AN OPTICAL MONITORING SYSTEM,” which is incorporated by reference in its entirety herein.

A controller of the optical blood monitoring system 14 uses the light intensities measured by the detectors to determine HCT values for blood flowing through the blood chamber 32. The controller calculates HCT, HGB, oxygen saturation, and change in blood volume (e.g., ABV) associated with blood passing through the blood chamber 32 to which the sensor device 34 is attached using a ratiometric model. The intensity of the received light at each of the various wavelengths is reduced by attenuation and scattering from the fixed intensity of the visible and infrared light emitted from each of the LED emitters. Beer's Law, for each wavelength of light, describes attenuation and scattering as follows:

iₙ=I₀₋ₙ [symbol] e^{-ε}^{ₚ}^{X}^{ₚ}^{d}^{ₚₜ} [symbol] e^{-ε}^{ₕ}^{X}^{ₕ}^{d}^{ₕ} [symbol] e^{-ε}^{ₚ}^{X}^{ₚ}^{d}^{ₚᵣ} Eq. (A)

where iₙ=received light intensity at wavelength n after attenuation and scattering; I₀₋ₙ=transmitted light intensity at wavelength n incident to the measured medium; e=the natural exponential term; ε=the extinction coefficient for the measured medium (p—blood chamber polycarbonate, b—blood); X=the molar concentration of the measured medium (p—blood chamber polycarbonate, b—blood); and d=the distance through the measured medium (pt—transmitting blood chamber polycarbonate, b—blood, pr—receiving blood chamber polycarbonate).

Since the properties of the polycarbonate blood chamber do not change, the first and third exponential terms in the above Eq. (A) are constants for each wavelength. Mathematically, these constant terms are multiplicative with the initial constant term I₀₋ₙ which represents the fixed intensity of the radiation transmitted from a respective LED emitter. For simplification purposes, Eq. (A) can be rewritten in the following form using bulk extinction coefficients and a modified initial constant I′₀₋ₙ as follows:

iₙ=I′₀₋ₙ [symbol] e^{-α}^{_{b}}^{d}^{_{b}} Eq. (B)

where iₙ=received light intensity at wavelength “n” after attenuation and scattering as though the detector were at the receive blood boundary; α=the bulk extinction coefficient (α_{b}=ε_{b}X_{b}) and I′₀₋ₙ=the equivalent transmitted light intensity at wavelength n as if applied to the transmit blood boundary accounting for losses through the blood chamber. Note that the term I′₀₋ₙ is the light intensity incident on the blood with the blood chamber losses included.

Using the approach defined in Eq. (B) above, the 810 nm wavelength which is isobestic for red blood cells and the 1300 nm wavelength which is isobestic for water can be used to determine the patient's hematocrit. The ratio of the normalized amplitudes of the measured intensity at these two wavelengths produces the ratio of the composite extinction values α for the red blood cells and the water constituents in the blood chamber, respectively. A mathematical function then defines the measured HCT value:

$$H C T = f [ \frac{ln ( \frac{{i}_{810}}{{I}_{0 ‐ 810}} )}{ln ( \frac{{i}_{1300}}{{I}_{0 ‐ 1300}} )} ] \qquad Eq . ( C )$$

where i₈₁₀ is the light intensity of the photo receiver at 810 nm, i₁₃₀₀ is the infrared intensity of the photodetector at 1300 nm and I₀₋₈₁₀ and I₀₋₁₃₀₀ are constants representing the intensity incident on the blood accounting for losses through the blood chamber. The above equation holds true assuming that the flow of blood through the blood chamber 32 is in steady state, i.e. steady pressure and steady flow rate.

The preferred function f[ ] is a second order polynomial having the following form:

$$H C T = f [ \frac{ln ( \frac{{i}_{810}}{{I}_{0 ‐ 810}} )}{ln ( \frac{{i}_{1300}}{{I}_{0 ‐ 1300}} )} ] = {A [ \frac{ln ( \frac{{i}_{810}}{{I}_{0 ‐ 810}} )}{ln ( \frac{{i}_{1300}}{{I}_{0 ‐ 1300}} )} ]}^{2} + B [ \frac{ln ( \frac{{i}_{810}}{{I}_{0 ‐ 810}} )}{ln ( \frac{{i}_{1300}}{{I}_{0 ‐ 1300}} )} ] + C . \qquad Eq . ( D )$$

A second order polynomial is normally adequate as long as the infrared radiation incident at the first and second wavelengths is substantially isobestic.

After the HCT value has been determined by a controller at the sensor device 34 or at the display device 35, the display device may be used to output the determined HCT value. Further, the controller may further determine an HGB concentration value based on the determined HCT value, with the HGB concentration value also being output on the display device 35.

For instance, the HGB for a blood sample corresponds to the mass of protein (e.g., in grams) for the blood sample, and an HGB concentration value corresponds to a protein mass per unit of blood sample volume. The HGB concentration value may be determined based on multiplying an HCT value and a mean corpuscular hemoglobin concentration (MCHC) value. It will be appreciated that the HCT value corresponds to the volume of red blood cells (RBCs) in a blood sample divided by the total volume of the blood sample, and that the MCHC value corresponds to an average mass of HGB per RBC divided by an average volume per RBC. It will further be appreciated that the MCHC value corresponds to mean corpuscular hemoglobin (MCH) divided by mean corpuscular volume (MCV), wherein MCH corresponds to an average mass of HGB per RBC of a patient (e.g., in picograms), and wherein MCV corresponds to an average volume per RBC of a patient (e.g., in femtoliters). Thus, when the HCT value is multiplied by the MCHC value, the HGB concentration value that is determined corresponds to a protein mass per unit of blood sample volume.

The medical system (e.g., hemodialysis system) depicted in FIG. 1 may be one of a plurality of medical systems in a dialysis clinic and/or in ICUs. Patients may come into the dialysis clinic for treatments at regular intervals, for example, on a Monday-Wednesday-Friday schedule or a Tuesday-Thursday-Saturday schedule. In some instances, the medical system depicted in FIG. 1 may be within a patient's home.

It will be appreciated that the medical system depicted in FIG. 1 is merely exemplary. The principles discussed herein may be applicable to other medical systems in which blood monitoring operations are performed.

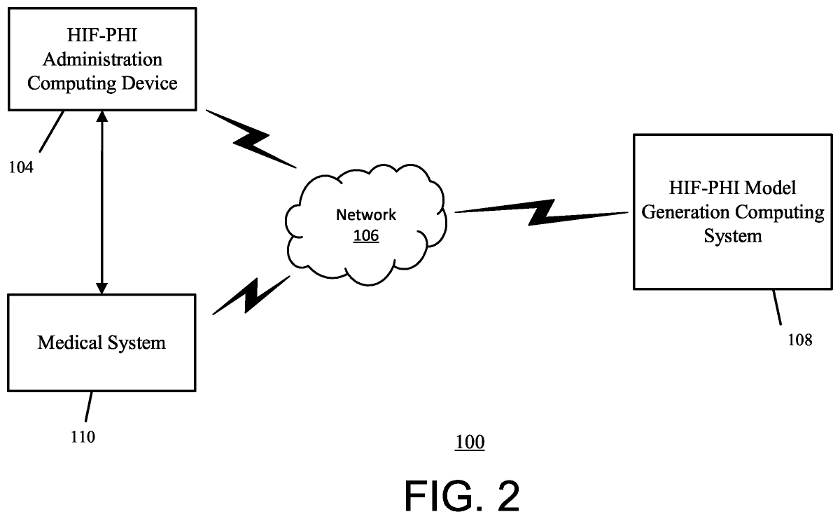

FIG. 2 is a simplified block diagram depicting an exemplary computing environment in accordance with one or more examples of the present application. The environment 100 includes a HIF-PHI administration computing device 104, a network 106, a HIF-PHI model generation computing system 108, and a medical system 110. Although the entities within environment 100 may be described below and/or depicted in the FIGs. as being singular entities, it will be appreciated that the entities and functionalities discussed herein may be implemented by and/or include one or more entities.

The entities within the environment 100 such as the HIF-PHI administration computing device 104, the HIF-PHI model generation computing system 108, and the medical system 110 may be in communication with other systems within the environment 100 via the network 106. The network 106 may be a global area network (GAN) such as the Internet, a wide area network (WAN), a local area network (LAN), or any other type of network or combination of networks. The network 106 may provide a wireline, wireless, or a combination of wireline and wireless communication between the entities within the environment 100. Additionally, and/or alternatively, one or more entities within the environment 100 may be in communication with each other without using the network 106. For instance, the HIF-PHI administration computing device 104 and the medical system 110 may be in communication with each other via one or more wireless protocols (e.g., WI-FI) and/or wired connections.

The medical system 110 may be the medical system depicted in FIG. 1 (e.g., the medical system 110 may be or include a dialysis/hemodialysis machine that performs dialysis treatment). The medical system 110 may provide and/or receive information from other entities within the environment 100 (e.g., the back-end computing system 108 and the user device 104).

In some instances, the medical system 110 may be and/or include another type of medical device such as another type of dialysis system. For instance, the medical system 110 may be a system for peritoneal dialysis in which a patient's peritoneal cavity is periodically infused with dialysate, and for which the membranous lining of the patient's peritoneum acts as a natural semi-permeable membrane that allows diffusion and osmosis exchanges to take place between the solution and the blood stream. In some examples, the medical system 110 may be optional. In such examples, the environment 100 may only include the HIF-PHI administration computing device 104 and the HIF-PHI model generation computing system 108.

The HIF-PHI model generation computing system 108 is a computing system that generates one or more HIF-PHI models. For example, the HIF-PHI model generation computing system 108 includes one or more computing devices, computing platforms, systems, servers, and/or other apparatuses capable of performing functions and/or actions such as generating one or more HIF-PHI models for HIF stabilizer treatment.

In some instances, the HIF-PHI model generation computing system 108 may, for example, communicate with the HIF-PHI administration computing device 104 and/or the medical system 110. For instance, the HIF-PHI model generation computing system 108 may provide the one or more generated HIF-PHI models to the HIF-PHI administration computing device 104 and/or the medical system 110. In some examples, the computing system 108 may include a display device that is configured to display information associated with the HIF stabilizer treatment.

The HIF-PHI model generation computing system 108 may be implemented using one or more computing platforms, devices, servers, and/or apparatuses. In some examples, the computing system 108 may include and/or be connected to a display device that is configured to display information associated with the HIF stabilizer treatment. In some variations, the HIF-PHI model generation computing system 108 may be implemented as engines, software functions, and/or applications. In other words, the functionalities of the HIF-PHI model generation computing system 108 may be implemented as software instructions stored in storage (e.g., memory) and executed by one or more processors.

The HIF-PHI administration computing device 104 may be and/or include, but is not limited to, a desktop, laptop, tablet, mobile device (e.g., smartphone device, or other mobile device), processor, controller, smart watch, an internet of things (IOT) device, or any other type of computing device that generally comprises one or more communication components, one or more processing components, and one or more memory components.

The HIF-PHI administration computing device 104 may receive the generated HIF-PHI models from the HIF-PHI model generation computing system 108. The HIF-PHI administration computing device 104 may use the generated HIF-PHI models to administer HIF stabilizer treatment. For example, initially a HIF-PHI dosage (e.g., a previous HIF-PHI dosage) may be prescribed to the patient. The HIF-PHI administration computing device 104 may obtain patient data for the patient, which includes previous HIF-PHI dosages. The HIF-PHI administration computing device 104 may input the patient data including the previous HIF-PHI dosage into a HIF-PHI model from the HIF-PHI model generation computing system 108 to determine whether the patient's hematocrit and/or hemoglobin concentrations are outside of a patient threshold. Based on the patient's hematocrit and/or hemoglobin concentrations being outside of the patient threshold, the HIF-PHI administration computing device 104 may determine a next HIF-PHI dosage for the patient using the HIF-PHI model. Then, the HIF-PHI administration computing device 104 may administer the next HIF-PHI dosage to the patient to adjust the patient's hematocrit and/or hemoglobin concentrations to be within the patient threshold. For example, the HIF-PHI administration computing device 104 may include and/or be connected to a display device and display the next HIF-PHI dosage to the anemia manager, the physician, the patient, and/or other operators that may issue a new prescription for the patient. For instance, the HIF-PHI medication may be administered orally. The computing system 108 and the computing device 104 may be configured to use a HIF-PHI model to determine a next HIF-PHI dosage to the patient and display the next HIF-PHI dosage. The patient may then orally take the HIF-PHI medication based on the next HIF-PHI dosage.

Additionally, and/or alternatively, the HIF-PHI model generation computing system 108 may determine one or more treatment schemes (e.g., decision trees) based on using one or more HIF-PHI models. The HIF-PHI model generation computing system 108 may provide the one or more treatment schemes to the HIF-PHI administration computing device 104. The HIF-PHI administration computing device 104 may determine and/or administer the next HIF-PHI dosage to the patient based on the received one or more treatment schemes. For example, the HIF-PHI administration computing device 104 may display the decision tree and/or the next HIF-PHI dosage for the patient on a display device associated with the HIF-PHI administration computing device 104.

In some instances, a singular entity may perform the functionalities of the HIF-PHI administration computing device 104 and the HIF-PHI model generation computing system 108. For instance, the HIF-PHI model generation computing system 108 may generate the HIF-PHI models and use the HIF-PHI models to determine the next HIF-PHI dosage as well as display the next HIF-PHI dosage to patients.

It will be appreciated that the exemplary environment depicted in FIG. 2 is merely an example, and that the principles discussed herein may also be applicable to other situations—for example, including other types of institutions, organizations, devices, systems, and network configurations.

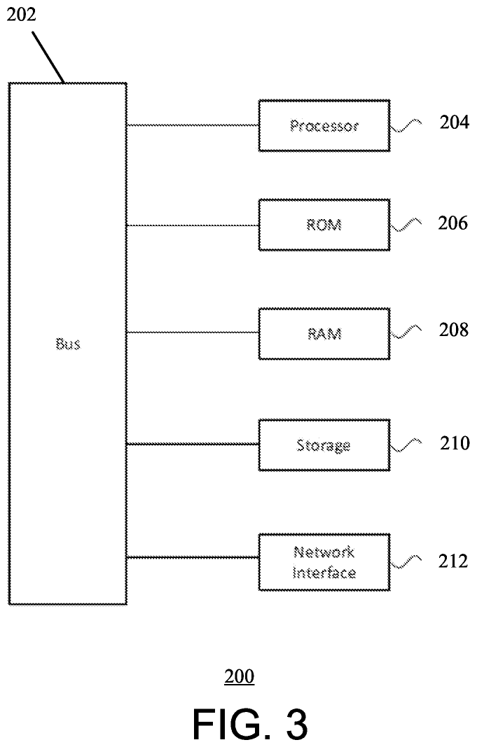

FIG. 3 is a simplified block diagram of one or more devices or systems within the exemplary environment of FIG. 2 according to one or more examples of the present application. For instance, the device/system 200 may be the HIF-PHI model generation computing system 108 and/or the HIF-PHI administration computing device 104 of FIG. 2. The device/system 200 includes a processor 204, such as a central processing unit (CPU), controller, and/or logic, that executes computer executable instructions for performing the functions, processes, and/or methods described herein. In some examples, the computer executable instructions are locally stored and accessed from a non-transitory computer readable medium, such as storage 210, which may be a hard drive or flash drive. Read Only Memory (ROM) 206 includes computer executable instructions for initializing the processor 204, while the random-access memory (RAM) 208 is the main memory for loading and processing instructions executed by the processor 204. The network interface 212 may connect to a wired network or cellular network and to a local area network or wide area network, such as the network 106. The device/system 200 may also include a bus 202 that connects the processor 204, ROM 206, RAM 208, storage 210, and/or the network interface 212. The components within the device/system 200 may use the bus 202 to communicate with each other. The components within the device/system 200 are merely exemplary and might not be inclusive of every component, server, device, computing platform, and/or computing apparatus within the device/system 200. Additionally, and/or alternatively, the device/system 200 may further include components that might not be included within every entity of environment 100.

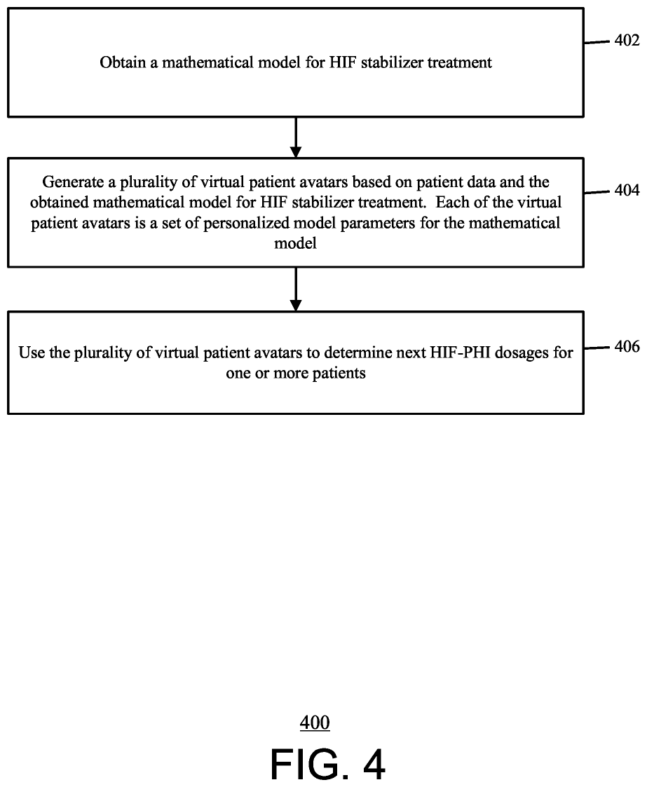

FIG. 4 is a flowchart of an exemplary process 400 for generating HIF-PHI models according to one or more examples of the present application. The process may be performed by a computing system such as the HIF-PHI model generation computing system 108 depicted in FIG. 2. However, it will be recognized that any of the following blocks may be performed in any suitable order, and that the process 400 may be performed in any suitable environment and by any suitable device or system. The descriptions, illustrations, and processes of FIG. 4 are merely exemplary and the process 400 may use other descriptions, illustrations, and processes for generating the HIF-PHI models.

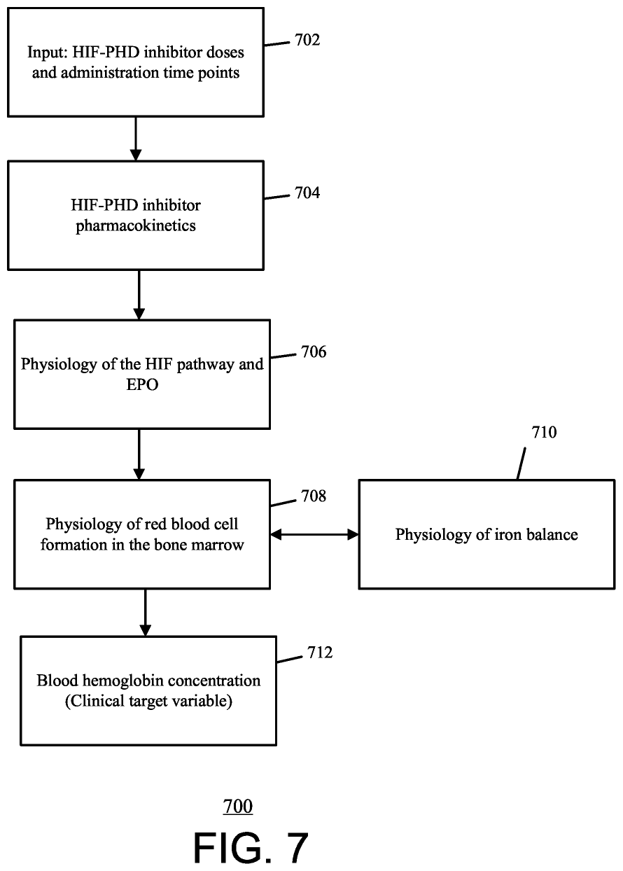

At block 402, the HIF-PHI model generation computing system 108 obtains a mathematical model for HIF stabilizer treatment. For example, the computing system 108 may receive the mathematical model from another entity (e.g., a server or computing system associated with an enterprise organization). Additionally, and/or alternatively, the mathematical model for the HIF stabilizer treatment may be stored in memory (e.g., external memory or memory within the computing system 108) and the computing system 108 may retrieve the mathematical model from the memory. FIG. 7 shows an exemplary mathematical model 700 for HIF stabilizer treatment that may be obtained by the HIF-PHI model generation computing system 108, and will be described in further detail below.

At block 404, the HIF-PHI model generation computing system 108 generates a plurality of virtual patient avatars based on population patient data (e.g., historical data and/or simulated data for a plurality of patients) and the obtained mathematical model for the HIF stabilizer treatment. Each of the virtual patient avatars is a set of personalized model parameters for the mathematical model. The population patient data may be historical data of a plurality of patients and/or simulated data of a plurality of patients. For example, the population patient data may include, but is not limited to, hemoglobin, hematocrit, HIF-PHI dosages (e.g., previous HIF-PHI dosages taken by the patients), iron doses, serum ferritin, transferrin saturation (TSAT), gender, height, weight, clinical parameters such as oxygen saturation, treatment time, bleeding events, blood transfusions, hospitalizations, and so on for a plurality of patients. The population patient data may be over a specified time window (e.g., a certain time period).

The computing system 108 may generate virtual patient avatars, which are a set of personalized model parameters, for the obtained mathematical model for the HIF stabilizer treatment. For example, the mathematical model, which is described in further detail in FIG. 7, includes a plurality of mathematical equations and patient parameters (e.g., variables). The computing system 108 may use the population patient data to determine patient parameters (e.g., variables) for the mathematical model. To put it another way, each patient and/or group of patients (e.g., a group of patients with similar patient characteristics such as weight, height, gender, and so on) may react slightly differently to HIF-PHI treatment. As such, a first patient and/or group of patients taking a certain HIF-PHI dosage may have different impacts (e.g., different hematocrit/hemoglobin concentration levels) from another patient and/or group of patients. The mathematical model may be associated with a first set of variables that are not patient/group specific and a second set of variables that are patient/group specific. The second set of variables may be the set of personalized model parameters that are indicated by the virtual patient avatars. Accordingly, the computing system 108 generates virtual patient avatars by inputting the population patient data into the mathematical model to determine the set of personalized model parameters.

The set of personalized model parameters associated with each virtual patient avatar may include, but are not limited to, the HIF-PHI bioavailability, the RBC lifespan, the parameters indicating patient-specific magnitude of the hemoglobin response to a given HIF-PHI dose, parameters indicating the patient's base hemoglobin levels in the absence of a pharmacologic therapy for anemia, and/or iron related parameters. The iron related parameters may include, but are not limited to parameters indicating the patient's hepcidin dynamics and its influence on iron availability and/or parameters indicating the patient's absorption of specific iron containing drugs.

For instance, the HIF-PHI bioavailability (e.g., the bioavailable fraction (f)) may be described in Eq. (1.5) below. And may be used to parameterize bioavailability of a patient. In particular, the computing system 108 may determine the bioavailable fraction (f) based on the absorption rate (α) and the clearance rate (η) in the gastrointestinal (GI) tract (before the HIF-PHI can be absorbed). The HIF-PHI bioavailability may indicate a fraction of the administered HIF-PHI dose that is bioactive (e.g., is not cleared from the body before absorption in the GI tract).

The RBC lifespan (κ_{ery}⁻¹) may be described in Eq. (1.17) below. The RBC lifespan may indicate the lifespan of red blood cells for the patients. For instance, the computing system 108 may determine the RBC lifespan based on the apoptosis rates for the cell population, the EPO for downregulation of progenitor apoptosis, and/or the rate of neocytolysis regulated by the hormone EPO. The RBC lifespan may indicate the average life span of a red blood cell.

The hemoglobin (HB) set point (c*_{hgb}) may be described in Eq. (1.6) and Eq. (1.7) below. For instance, the computing system 108 may determine the HB set point based on the blood hemoglobin concentration as measured by the kidneys, the number of erythrocytes, activation and inhibition functions, the hypoxic upregulation of the HIF signal in response to decreased in the blood hemoglobin concentration, the upregulation of the HIF signal in response to HIF-PHI administrations, the decay rate of the HIF signal, and/or other factors. The HB set point may be defined as the blood HB concentration at which the activity of endogenous feedbacks such as upregulating or downregulating RBC generation is minimal.

The basal EPO synthesis rate (βₑ) may be described in Eq. (1.8) below. For instance, the computing system 108 may determine the basal EPO synthesis rate based on the serum EPO concentration, the EPO synthesis, and/or the EPO decay rate. The basal EPO synthesis rate may indicate the rate at which EPO is synthesized in the body.

The HIF signal threshold (s̅) may be described in Eq. (1.6) below. For instance, the computing system 108 may determine the HIF signal threshold based on the blood hemoglobin concentration as measured by the kidneys, the number of erythrocytes, activation and inhibition functions, the hypoxic upregulation of the HIF signal in response to decreased in the blood hemoglobin concentration, the upregulation of the HIF signal in response to HIF-PHI administrations, the decay rate of the HIF signal, and/or other factors. The HIF signal threshold may indicate the amount of bioactive HIF-PHI needed to elicit half its maximum effect on gain in HIF signaling activity, and the HIF signal in the model is an abstract variable indicating activity of the HIF signaling pathway.

The hepcidin decay rate (κₚ) may be described in Eq. (1.28) and/or Eq. (1.29) below. For instance, the computing system 108 may determine the hepcidin decay rate based on the residual renal function of the patient. The hepcidin decay rate may indicate the decay rate of hepcidin within a patient's body.

As such, each virtual patient avatar may indicate a set of personalized model parameters, and the computing system 108 may generate a plurality of these virtual patient avatars. To generate the virtual patient avatars, the computing system 108 may systematically fit (e.g., assign) a specified set of model parameters to the population patient data using inputs (e.g., previous HIF-PHI dosages). Then, the computing system 108 determines the “goodness” of a fit based on deviations between the simulated and measured hemoglobin/hematocrit concentrations (e.g., between the hemoglobin/hematocrit concentrations output by the HIF-PHI model using the assigned set of model parameters and the actual hemoglobin/hematocrit concentrations from the population patient data). The computing system 108 may use a cost function to define the goodness of the fit, and the fitting procedure may be performed using one or more parameter fit algorithms. In some examples, other pairs of simulated and measured data elements (e.g., HIF-PHI serum concentrations, erythropoietin serum concentrations, iron plasma concentrations, and so on) may also be used to determine the goodness of the fit.

In other words, the computing system 108 may determine a subset of population patient data based on certain characteristics (e.g., height, weight, gender, and so on). The computing system 108 may assign values to the set of model parameters described above (e.g., the HIF-PHI bioavailability, RBC lifespan, hemoglobin set point, basal EPO synthesis rate, HIF signal threshold, hepcidin decay rate, and/or additional model parameters). Subsequently, the computing system 108 may determine previous HIF-PHI dosages and hemoglobin/hematocrit concentrations within the subset of population patient data. Then, the computing system 108 may input the previous HIF-PHI dosages (e.g., via time-dependent administrations of HIF-PHIs, Γₒᵣₐₗ^{s}(t) from Eq. (1.1) below where Γₒᵣₐₗ^{s}(t)=ΣᵢHᵢδ(t−tᵢ) and i refers to the i-th dose administration, Hᵢ is the administered dose, δ is the Dirac delta function, and tᵢ is the time point of administration) and the assigned set of model parameters into the mathematical model to determine simulated hemoglobin/hematocrit concentrations. The computing system 108 may compare the simulated hemoglobin/hematocrit concentrations with the actual hemoglobin/hematocrit concentrations to determine deviations between the two. Afterwards, the computing system 108 may assign new values to the set of model parameters and repeat the process. The computing system 108 may continuously assign new values to the set of model parameters and determine deviations between the simulated hemoglobin/hematocrit concentrations with the actual hemoglobin/hematocrit concentrations. For instance, the computing system 108 may use the previously computed deviations to determine which new values to try in the next iteration. In other words, the computing system 108 may use one or more parameter fit algorithms (e.g., optimization and/or other types of comparison algorithms such as distance algorithms that compare distances between the simulated and actual concentrations) to determine the best set of model parameters for the subset of population patient data. The computing system 108 may determine the set of model parameters for the virtual patient avatar as the best set of model parameters. For instance, the computing system 108 may compare the distance between the simulated and actual hemoglobin/hematocrit concentrations and use the set of model parameters that produced the smallest distance between the simulated and actual concentrations.

The computing system 108 may determine a plurality of virtual patient avatars based on using subsets of the population data and each of the plurality of virtual patient avatars may be associated with certain patient characteristics (e.g., gender, weight, height) and a set of personalized model parameters.

In some examples, in addition to inputting the previous HIF-PHI dosages and the assigned set of model parameters into the mathematical model, the computing system 108 may further input ferritin concentrations into the mathematical model to determine simulated hemoglobin/hematocrit concentrations. For instance, the computing system 108 may obtain ferritin concentrations from the subset of the population patient data, and input the obtained ferritin concentrations into the mathematical model via Eq. (1.27) below. Additionally, and/or alternatively, the mathematical model may include a ferritin equation (e.g., Eq. (1.37) below) and the computing system 108 may compute the ferritin concentrations using the ferritin equation.

In some instances, the computing system 108 may perform pre-calibrating functions prior to generating the virtual patient avatars. For instance, the computing system 108 may determine blood volumes based on the subset of population patient data. For example, using the gender, height, weight, and/or other information from the subset of population patient data as well as Eq. (1.38) below, the computing system 108 may determine a blood volume for the virtual patient avatar. Then, using the blood volume, the computing system 108 may determine rates such as formation rates, synthesis rates, and/or other rates for the virtual patient avatar. For instance, the computing system 108 may determine the HIF signal gain rate (shown as βₕ in Eq. (1.6) below), the EPO synthesis rate (shown as βₑ in Eq. (1.8) below), the progenitor formation rate (shown as β_{prog} in Eq. (1.9) below), and/or the hepcidin synthesis rate (shown as βₚ in Eqs. (1.25) and (1.26) below). The computing system 108 may use the determined rates, the ferritin concentrations, the assigned set of model parameters, the previous HIF-PHI dosages, the mathematical model, and/or other data to determine simulated hemoglobin/hematocrit concentrations. Then, based on the simulated hemoglobin/hematocrit concentrations, the computing system 108 may determine a best set of model parameters to use for the virtual patient avatar.

In some variations, in addition to the simulated hemoglobin/hematocrit concentrations, the computing system 108 may further use the mathematical model, the previous HIF-PHI dosages, and/or other information (e.g., ferritin concentrations) to determine simulated serum levels for the HIF-PHI (shown and described as the variable “s” in Eqs. (1.1)-(1.4) below), the EPO (shown as the variable “e” in Eq. (1.8)), the hepcidin (shown as the variable “p” in Eqs. (1.25) or (1.26), the iron, and/or other simulated data points. The computing system 108 may further obtain the actual serum levels for the HIF-PHI, the EPO, the hepcidin, the iron, and/or other data points from the subset of population patient data. The computing system 108 may compare the simulated and actual data points, and use the one or more parameter fit algorithms to determine the best set of model parameters to use for the virtual patient avatar.

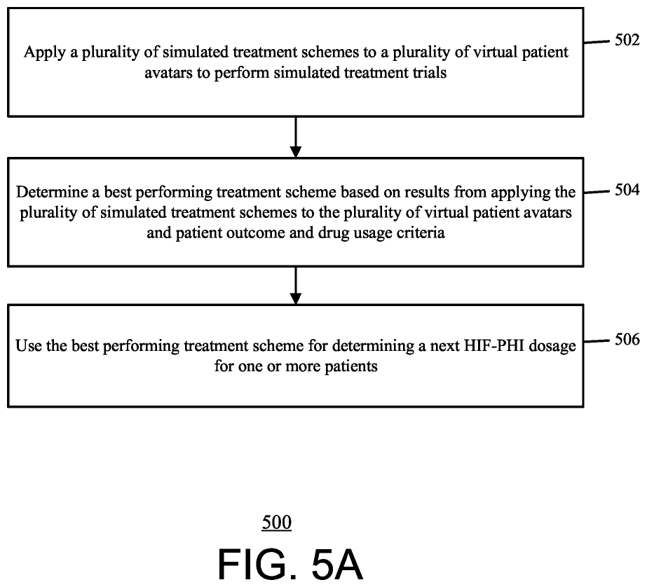

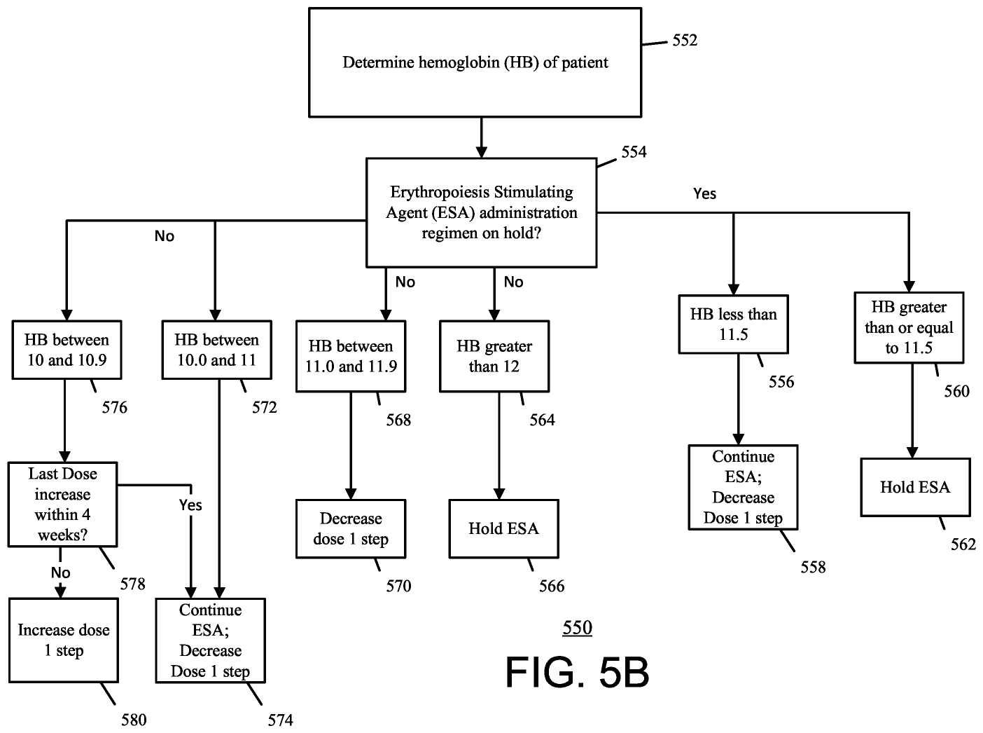

At block 406, the HIF-PHI model generation computing system 108 uses the plurality of virtual patient avatars to determine next HIF-PHI dosages for one or more patients. For example, the computing system 108 may determine a next HIF-PHI dosage indicating an amount of HIF-PHI medication for the patient to take based on the virtual patient avatars. In some instances, the computing system 108 may determine one or more treatment schemes or protocols based on the virtual patient avatars. The treatment scheme may indicate a next HIF-PHI dosage based on a patient's hemoglobin concentration. Each virtual patient avatar may be associated with a particular treatment scheme or a particular treatment scheme may be associated with two or more patient avatars. A treatment scheme relates to a fully-fledged general HIF-PHI treatment algorithm for patients. In some instances, the treatment scheme may be a decision tree indicating how to change a current HIF-PHI prescription based on recent measurements (e.g., “increase dose due to falling hemoglobin levels”). Additionally, and/or alternatively, the treatment scheme may indicate different drug doses (HIF-PHI and/or iron), different treatment schedules (e.g. daily, 3×/week, weekly), and/or different administration routes (e.g. oral vs intravenous). FIGS. 5A and 5B will describe generating treatment schemes and determining next HIF-PHI dosages for patients using the generated treatment schemes.

FIG. 5A is a flowchart of an exemplary process for determining a next HIF-PHI dosage for a patient based on using a treatment scheme according to one or more examples of the present application. The process may be performed by a computing device such as the HIF-PHI administration computing device 104 and/or the HIF-PHI model generation computing system 108 depicted in FIG. 2. However, it will be recognized that any of the following blocks may be performed in any suitable order, and that the process 500 may be performed in any suitable environment and by any suitable device or system. The descriptions, illustrations, and processes of FIG. 5A are merely exemplary and the process 500 may use other descriptions, illustrations, and processes for adjusting a patient's hematocrit and/or hemoglobin concentrations.

At block 502, the computing system 108 applies a plurality of simulated treatment schemes to a plurality of virtual patient avatars to perform simulated treatment trials. For instance, as mentioned above, the computing system 108 may generate a plurality of virtual patient avatars (e.g., a set of personalized model parameters for the mathematical model for the HIF stabilizer treatment) and store the plurality of virtual patient avatars. The computing system 108 may use the plurality of virtual patient avatars to conduct virtual clinical trials and assess one or more treatment schemes.

For instance, a treatment scheme relates to a fully-fledged general HIF-PHI treatment algorithm for patients. In some instances, the treatment scheme may be a decision tree indicating how to change a current HIF-PHI prescription based on recent measurements (e.g., “increase dose due to falling hemoglobin levels”). FIG. 5B shows an exemplary treatment scheme according to one or more examples of the present application. In particular, FIG. 5B shows a treatment scheme 550 that uses the hemoglobin concentrations of the patient to determine the next HIF-PHI dosage for the patient.

For example, at block 552, hemoglobin (HB) of the patient is determined. At block 554, whether erythropoiesis stimulating agent (ESA) administration regimen on hold is determined. If yes, treatment scheme 550 moves to block 556 or 560. If HB is less than 11.5 then, treatment scheme 550 moves to block 558. If HB is greater than or equal to 11.5, then treatment scheme 550 moves to block 562. At block 562, the treatment scheme 550 holds the ESA. In other words, the prescription of the patient indicates to hold the ESA regimen. At block 558, the treatment scheme 550 continues ESA (e.g., the ESA regimen is continued for the patient) and the dosage for HIF-PHI is decreased by one step. For instance, the HIF-PHI dosage may be based on a plurality of steps (e.g., 11 steps). At step 1, the HIF-PHI dosage is on hold (e.g., no HIF-PHI dosage for the patient). At step 2, the HIF-PHI dosage may be indicated as 10 milligrams (mg) daily. At step 3, the HIF-PHI dosage may be 20 mg daily. At step 4, the HIF-PHI dosage may be 30 mg daily. At step 5, the HIF-PHI dosage may be 40 mg daily. At step 6, the HIF-PHI dosage may be 50 mg daily. At step 7, the HIF-PHI dosage may be 60 mg daily. At step 8, the HIF-PHI dosage may be 80 mg daily. At step 9, the HIF-PHI dosage may be 100 mg daily. At step 10, the HIF-PHI dosage may be 130 mg daily. At step 11, the HIF-PHI dosage may be 150 mg daily. As such, at block 558 (e.g., based on the ESA being on hold and HB less than 11.5), the treatment scheme 550 may indicate to decrease the HIF-PHI dosage by 1 step (e.g., if the patient was at step 7, then the treatment scheme 550 may indicate to decrease the patient to step 6 and the next HIF-PHI dosage for the patient may be 50 mg daily.

If the ESA is not on hold, then the treatment scheme 550 checks blocks 564, 568, 572, and 576. Based on the HB being greater than 12, then the treatment scheme 550 indicates to hold the ESA at block 566. Based on the HB being between 11.0 and 11.9, then the treatment scheme 550 indicates to decrease dose 1 step at block 570. Based on the HB being between 10.0 and 11, the treatment scheme 550 indicates to continue ESA and decrease the HIF-PHI dosage 1 step. Based on the HB between 10 and 10.9, the treatment scheme 550 checks whether the last dose increase was within 4 weeks at block 578. If yes, then the treatment scheme 550 moves to block 574 and the ESA is continued and the HIF-PHI dosage is decreased 1 step. If no, then the treatment scheme 550 moves to block 580 and the HIF-PHI dosage is increased by 1 step.

The computing system 108 may generate a plurality of simulated treatment schemes such as treatment scheme 550. As mentioned above, treatment scheme 550 is merely exemplary and the computing system 108 may generate a plurality of different treatment schemes for block 502. For instance, the plurality of treatment schemes may include treatment scheme 550 as well as other treatment schemes such as by removing blocks from treatment scheme 550 (e.g., removing block 578 and having block 576 connect directly to block 580), adding additional blocks to treatment scheme 550 (e.g., adding new branches or decision blocks prior to determining to increase or decrease the HIF-PHI dosage), and/or modifying the blocks from treatment scheme 550 (e.g., changing the ranges for the HB within blocks 556, 560, 564, 568, 572, and/or 576). Additionally, and/or alternatively, the treatment scheme blocks may indicate and/or be associated with recommendations in certain or special situations such as, but not limited to, severe declines or inclines in HGB (e.g., HGB changes of greater than 1 gram/deciliter (dl) in two weeks), situations of very low HGB (e.g., less than 8 gram/dl), ferritin or TSAT below or above certain thresholds, or exceptions such as patient recently returned from hospital, no HGB measurements available for a certain number of weeks, incorrect dose administration, and/or patient's therapy was on hold for a certain number of weeks.

In some instances, the computing system 108 may generate the treatment schemes automatically and/or based on received user input. For example, a user may provide input to create the treatment schemes. Additionally, and/or alternatively, the computing system 108 may generate the entire treatment schemes and/or portions of the treatment scheme.

After obtaining the plurality of simulated treatment schemes (e.g., treatment scheme 550), the computing system 108 may apply the treatment schemes to the virtual patient avatars to perform simulated treatment trials. For instance, each virtual patient avatar includes and/or is associated with a set of personalized model parameters (e.g., the HIF-PHI bioavailability, the RBC lifespan, the parameters indicating patient-specific magnitude of the hemoglobin response to a given HIF-PHI dose, parameters indicating the patient's base hemoglobin levels in the absence of a pharmacologic therapy for anemia, parameters indicating the patient's hepcidin dynamics and its influence on iron availability, and/or parameters indicating the patient's absorption of specific iron containing drugs). The computing system 108 may use the personalized model parameters for the mathematical model described in FIG. 7. For example, the virtual patient avatar may indicate values for HIF-PHI bioavailability, the RBC lifespan, and other personalized model parameters. The computing system 108 may determine a HIF-PHI model based on those values and the mathematical model. In other words, the HIF-PHI model may be mathematical model shown in FIG. 7 with values (e.g., an RBC average lifespan) indicated by the virtual patient avatar (e.g., values for the HIF-PHI bioavailability and/or the RBC lifespan). Furthermore, the computing system 108 may use the population patient data and the HIF-PHI model for the virtual patient avatar to assess the treatment scheme 550. For instance, based on the HB concentration of the patient, the computing system 108 may determine the next HIF-PHI dosage for the patient (e.g., maintain, increase, or decrease the HIF-PHI dosage indicated by blocks 580, 574, 570, 566, 558, 562). The computing system 108 may then use the next HIF-PHI dosage along with the HIF-PHI model to determine the expected hematocrit/hemoglobin concentration for the patient. For instance, the computing system 108 may determine an output of the HIF-PHI model based on the next HIF-PHI dosage indicated by the treatment scheme (e.g., use Eq. (1.7) to determine the expected hematocrit/hemoglobin concentrations for the patient).

The computing system 108 may continue to determine expected hematocrit/hemoglobin concentrations using the same treatment scheme, the HIF-PHI model, and the population patient data. Also, the computing system 108 may assess different treatment schemes (e.g., determine expected hematocrit/hemoglobin concentrations) based on using the population patient data and the HIF-PHI model. Each simulated treatment trial may be associated with a virtual patient avatar (e.g., a HIF-PHI model) and a particular treatment scheme (e.g., treatment scheme 550). The results of the simulated treatment trials be based on the results from the HIF-PHI model (e.g., the expected hematocrit/hemoglobin concentrations output by the HIF-PHI model) and/or the simulated therapy (e.g., a time series of administered HIF-PHI doses as determined by the simulated treatment scheme. The results of the simulated treatment trials may indicate and/or be associated with a HB time series and/or a HIF-PHI administration time series (e.g., the results may be based on the decisions of the tested treatment algorithm and the HB dynamics of the virtual patient avatars).

At block 504, the computing system 108 determines a best performing treatment scheme based on results from applying the plurality of simulated treatment schemes to the plurality of virtual patient avatars and patient outcome and drug usage criteria. For instance, after applying the treatment schemes to the virtual patient avatars, the computing system 108 may determine a plurality of results (e.g., the expected hematocrit/hemoglobin concentrations output by the HIF-PHI model). The computing system 108 may then assess which treatment schemes performed best given the physiological heterogeneity of the virtual patient avatar. For instance, the computing system 108 may evaluate the outputs (e.g., results) by performing statistical analysis. For example, the computing system 108 may determine the best treatment scheme according to specific criteria (e.g., the treatment scheme leading to the longest average time spent within a set hemoglobin target). In some instances, in order to perform such simulated trials, real-world aspects affecting the performance of a treatment algorithm (e.g., compliance, random events like missed treatments, lab sample shipment delays, and so on) may be considered as well and may be part of the simulated therapy. This is described further in U.S. patent application Ser. No. 14/974,861, titled “SYSTEM AND METHOD OF CONDUCTING IN SILICO CLINICAL TRIALS”, which is incorporated by reference herein in its entirety.

In some variations, the specific criteria to evaluate the “best treatment” scheme include, but are not limited to, the number of patients within a target threshold, time patients spend within target, number of patients outside target, number of patients with very high/or very low hemoglobin levels, number of patients that would require a blood transfusion, amount of drug administered (HIF and/or iron), hemoglobin variability (inter- and intra individual), average HIF-PHI usage during a time interval, and/or other criteria.

In some examples, each virtual patient avatar may be associated with a particular treatment scheme. In other examples, two or more virtual patient avatars, including all of the patient avatars, may be associated with a treatment scheme. For example, one or more medical clinics and/or the entire enterprise organization may use a single treatment scheme. The computing system 108 may assess the plurality of treatment schemes by applying a plurality of HIF-PHI models associated with a plurality of virtual patient avatars to determine results from the simulated treatment trials. Based on the results, the computing system 108 may determine a best performing treatment scheme and use the best performing treatment scheme for the medical clinic and/or the entire enterprise organization. For instance, treatment scheme 550 may be determined to be the best performing treatment scheme and used for the medical clinic and/or the entire enterprise organization.

In some variations, the computing system 108 may assess each of the plurality of treatment schemes over a time period or time window. In other words, the computing system 108 may perform multiple iterations of a treatment scheme to determine results from the treatment scheme (e.g., multiple hematocrit and/or hemoglobin concentrations for the patients and the HIF-PHI dosages given to the patients during each iteration). The computing system 108 may determine the best treatment scheme (e.g., the number of patients within target, time patients spend within target, number of patients outside target, number of patients with very high/or very low hemoglobin levels, and/or average HIF-PHI usage during the time interval) based on assessing the treatment schemes over the time period or window.

At block 506, the computing system 108 uses the best performing treatment scheme for determining a next HIF-PHI dosage for one or more patients. For example, the computing system 108 may administer the next HIF-PHI dosage by determining the next HIF-PHI dosage based on a hematocrit and/or hemoglobin concentration of the patient. For instance, the computing system 108 may determine the best performing treatment scheme is treatment scheme 550 and use the patient's hematocrit and/or hemoglobin concentration and/or one or more previous HIF-PHI dosages of the patient to determine the next HIF-PHI dosage (e.g., increase or decrease dose 1 step or maintain dose). The computing system 108 may be connected to and/or include a display device that displays the next HIF-PHI dosage.

In some instances, the computing system 108 may provide the best performing treatment scheme to the HIF-PHI administration computing device 104, and the computing device 104 may administer the next HIF-PHI dosage by determining the next HIF-PHI dosage and displaying the next HIF-PHI dosage.

In some examples, the computing system 108 may update and/or continuously update the best performing treatment schemes. For instance, based on the best performing treatment scheme (e.g., treatment scheme 550), the computing system 108 may generate additional treatment schemes. For example, the computing system 108 may modify, add, or remove blocks from the best performing treatment scheme to determine a plurality of new treatment schemes and then use process 500 to assess the new treatment schemes. The computing system 108 may then determine the best performing treatment scheme from the new treatment schemes.

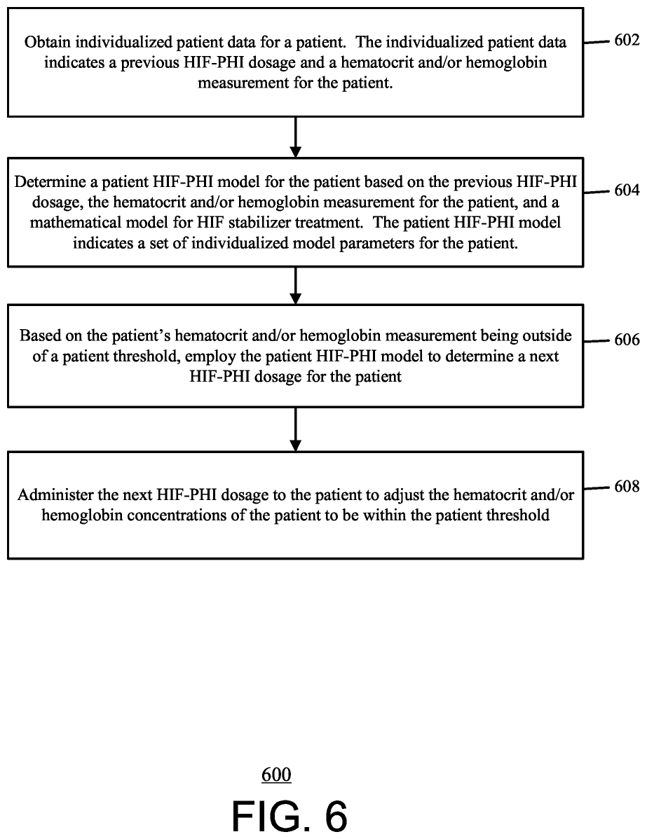

FIG. 6 is a flowchart of an exemplary process for adjusting a patient's hematocrit and/or hemoglobin concentration using a HIF-PHI model according to one or more examples of the present application. The process may be performed by a computing device such as the HIF-PHI administration computing device 104 and/or the HIF-PHI model generation computing system 108 depicted in FIG. 2. However, it will be recognized that any of the following blocks may be performed in any suitable order, and that the process 600 may be performed in any suitable environment and by any suitable device or system. The descriptions, illustrations, and processes of FIG. 6 are merely exemplary and the process 600 may use other descriptions, illustrations, and processes for adjusting a patient's hematocrit and/or hemoglobin concentrations.

For example, in some variations, a computing device (e.g., the computing system 108 and/or the computing device 104) may obtain individualized patient data for one or more patients (e.g., a particular patient's previous HIF-PHI dosages, hematocrit/hemoglobin concentrations, gender, weight, and/or other characteristics of the patient). The computing device may determine a patient specific HIF-PHI model (e.g., a patient HIF-PHI model) for the patient based on the individualized patient data (e.g., patient-individual data). To put it another way, initially, the computing system 108 may determine a treatment scheme and use the treatment scheme to determine HIF-PHI dosages for the patient. During the HIF stabilizer treatment, the clinic may take blood draws and/or perform other tests (e.g., weekly test data) to determine patient data that is specific to the patient. The computing system 108 may then use process 600 to determine a patient specific HIF-PHI model for the patient based on the individualized patient data, and may use the patient HIF-PHI model to determine next HIF-PHI dosages for the patient.

Additionally, and/or alternatively, process 600 may be performed separately from processes 400 and 500. In other words, the computing system 108 may obtain the individualized patient data and generate the patient HIF-PHI model without initially using a treatment scheme to determine HIF-PHI dosages for the patient. For instance, in some variations, the patient data may be static (e.g., the computing system 108 might not continuously receive updates to the patient data or may rarely receive updates to the patient data) and the computing system 108 may use process 400 and 500 to determine the next HIF-PHI dosages for patients. In other variations, the patient data may be dynamic (e.g., the computing system 108 may frequently receive updates to the patient data) and the computing system 108 may use process 600 alone or in combination with processes 400 and 500.

At block 602, the computing device (e.g., the HIF-PHI administration computing device 104 and/or the HIF-PHI model generation computing system 108) obtains individualized patient data for a patient. The individualized patient data indicates a previous HIF-PHI dosage and a hematocrit and/or hemoglobin measurement for the patient. The previous HIF-PHI dosage may be a dosage prescribed to the patient for HIF stabilizer treatment (e.g., using a treatment scheme such as treatment scheme 550 and/or determined by a clinician). Additionally, and/or alternatively, the individualized patient data may include, but is not limited to, include, but is not limited to, hemoglobin, hematocrit, HIF-PHI dosages (e.g., previous HIF-PHI dosages taken by the patients), iron doses, serum ferritin, low transferrin saturation (TSAT), gender, height, weight, clinical parameters such as oxygen saturation, treatment time, bleeding events, blood transfusions, hospitalizations, and so on for the patient.

At block 604, the computing device determines a patient HIF-PHI model for the patient based on the previous HIF-PHI dosage, the hematocrit and/or hemoglobin measurement for the patient, and a mathematical model for HIF stabilizer treatment (e.g., the mathematical model described below in FIG. 7). The patient HIF-PHI model indicates a set of individualized model parameters for the patient. For example, referring to block 404 of FIG. 4, the computing system 108 generates virtual patient avatars indicating a set of personalized model parameters based on population patient data. At block 604, rather than using population patient data associated with a plurality of patients, the computing device determines a set of personalized model parameters based on individualized patient data for a particular patient. The set of parameters for the patient may include, but are not limited to, the HIF-PHI bioavailability, the RBC lifespan, the parameters indicating patient-specific magnitude of the hemoglobin response to a given HIF-PHI dose, parameters indicating the patient's base hemoglobin levels in the absence of a pharmacologic therapy for anemia, and/or iron related parameters. Based on the set of personalized model parameters and the mathematical model, the computing device may determine a patient HIF-PHI model for the patient.

In some examples, the computing device may use the previous HIF-PHI dosage (e.g., historical HIF-PHI administration data points (Γₒᵣₐₗ^{s}(t))) and/or other information (e.g., ferritin levels, and/or patient characteristics such as gender, height weight, blood volume, and so on) to determine a patient HIF-PHI model.

In some variations, the computing device may select the patient HIF-PHI model from one of the plurality of virtual patient avatars determined at block 404. For instance, based on comparing the individualized patient data with subsets of population patient data, the computing device may determine the virtual patient avatar that most resembles the patient based on the individualized patient data (e.g., the virtual patient avatar that has the same or similar patient characteristics such as height, weight, gender, previous HIF-PHI dosages, hemoglobin concentration measurements, and so on).

At block 606, based on the patient's hematocrit and/or hemoglobin concentrations being outside of a patient threshold, the computing device employs the patient HIF-PHI model to determine a next HIF-PHI dosage for the patient. For instance, the computing device may obtain the patient's hematocrit and/or hemoglobin concentrations from the individualized patient data and compare these measurements with a patient threshold (e.g., a patient threshold set by a clinician). Based on the patient's hematocrit and/or hemoglobin concentrations being outside of the patient threshold (e.g., above or below the threshold), the computing device may employ the patient HIF-PHI model to determine a next HIF-PHI dosage for the patient. For example, the computing device may input a plurality of different HIF-PHI dosages into the patient HIF-PHI model (e.g., via Eq. (1.1) below) and the patient HIF-PHI model may provide outputs such as expected hematocrit/hemoglobin concentrations based on the input HIF-PHI dosage. The computing device may determine the next HIF-PHI dosage based on the outputs from the patient HIF-PHI model. For instance, the computing device may select a HIF-PHI dosage as the next HIF-PHI dosage based on the associated output (e.g., an expected hematocrit and/or hemoglobin concentration) from the patient HIF-PHI model being within the patient threshold. Additionally, and/or alternatively, the computing device may select the HIF-PHI dosage based on the HIF-PHI dosage being the least amount of HIF-PHI amount that causes the expected output from the patient HIF-PHI model to be within the patient threshold (e.g., if multiple HIF-PHI dosages causes the output to be within the patient threshold, the computing device may select the lowest amount of HIF-PHI dosages from the multiple HIF-PHI dosages). Additionally, and/or alternatively, the computing device may use other factors to select the HIF-PHI dosage for the patient.

At block 608, the computing device administers the next HIF-PHI dosage to the patient to adjust the hematocrit and/or hemoglobin concentrations to be within the patient threshold. For example, the computing device may cause display of the next HIF-PHI dosage on a display device connected to or included within the computing device. In some instances, the computing device may cause display of a plurality of HIF-PHI dosages output from the patient HIF-PHI model and a user (e.g., clinician) may select the next HIF-PHI dosage for the patient from the plurality of HIF-PHI dosages (e.g., the plurality of HIF-PHI dosages that caused the patient's expected hematocrit/hemoglobin concentrations to be within the patient threshold). In other instances, the computing device may display a single HIF-PHI dosage for the patient.

In some examples, the computing device may determine the patient threshold based on user input. For instance, a doctor, nurse, clinician, and/or other member may provide user input indicating the patient threshold (e.g., a specified hematocrit/hemoglobin concentration range).

In some variations, process 600 may repeat. For example, after a certain time interval (e.g., each week), the computing device may obtain new patient data. For example, the patient may provide blood samples every so often (e.g., weekly, bi-weekly, or monthly). The computing device may receive new data indicating lab results after each cycle, and may update the HIF-PHI model and/or generate a new HIF-PHI model for the patient based on the new data. The computing device may employ the new HIF-PHI model to determine the next HIF-PHI dosages for the patient.

FIG. 7 is a block diagram of an exemplary mathematical model 700 for the HIF stabilizer treatment according to one or more examples of the present application. As mentioned previously, the mathematical model 700 may be used to determine/generate the plurality of virtual patient avatars, adjust the HIF-PHI dosage to the patient to adjust the patient's hematocrit/hemoglobin concentrations, and/or assess a plurality of simulated treatment schemes.

For example, at block 702, the input to the mathematical model 700 includes HIF-PHD inhibitor doses and/or administration time points. In some instances, the input to the mathematical model 700 may include additional information.

Block 704 of the mathematical model 700 is the HIF-PHD inhibitor pharmacokinetics. For instance, from a biology standpoint, the HIF-PHIs may be administered orally. The pharmacokinetic trajectories of the serum concentration of HIF-PHIs show a typical one-peak structure with a quickly decaying and a slowly decaying component. Hence, the pharmacokinetic part of the model 700 model follows a dynamic of dose uptake and absorption into a compartment with fast clearance and leakage into a compartment with slow clearance. In total, this is described by three variables, which denote the HIF-PHI mass in the relevant part of the gastro-intestinal tract (\tilde{s}), the fast compartment (s₁) and the slow compartment (s₂). This is described in Eqs. (1.1), (1.1a), (1.2), and (1.3) below.

$$\frac{d \tilde{s}}{dt} = {Γ}_{oral}^{s} ( t ) - a \tilde{s} - η \tilde{s} , \qquad Eq . ( 1.1 )$$

$${Γ}_{oral}^{s} ( t ) = {\sum }_{i} {H}_{i} δ ( t - {t}_{i} ) \qquad Eq . ( 1.1 a )$$

$$\frac{{ds}_{1}}{dt} = α \tilde{s} - {ω}_{1 \to {2}^{{s}_{1}}} - {k}_{1} {s}_{1} \qquad Eq . ( 1.2 )$$

$$\frac{{ds}_{2}}{dt} = {ω}_{1 \to {2}^{{s}_{1}}} - {k}_{2} {s}_{2} \qquad Eq . ( 1.3 )$$

Above, Γₒᵣₐₗ^{s}(t) describes the time-dependent administrations of HIF-PHIs, α denotes the absorption rate, η denotes the clearance rate in the GI tract (before the HIF-PHI can be absorbed) and k₁ and k₂ denote the clearance rates after absorption in the fast and slow compartment, respectively. ω_{1→2} denotes the transition rate from the fast to the slow compartment. i refers to the i-th dose administration, Hᵢ is the administered dose, δ is the Dirac delta function, and tᵢ is the time point of administration. The total biologically active HIF-PHI mass, which is denoted by s, is defined by Eq. (1.4) below.

s=s₁+s₂ Eq. (1.4)

The bioavailable fraction of HIF-PHI, which is denoted by f, is provided by Eq. (1.5) below.

f=α/(α+η) Eq. (1.5)

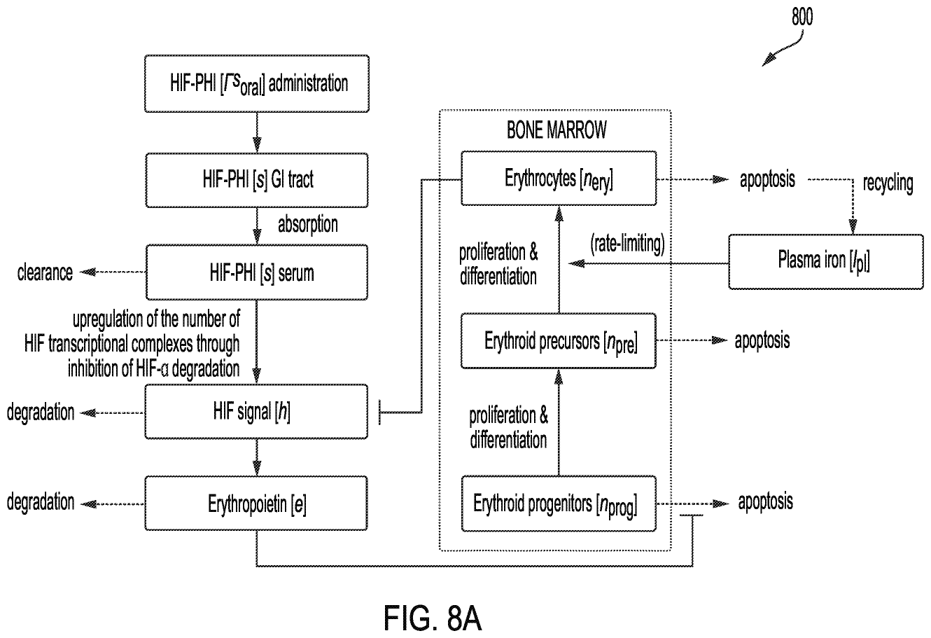

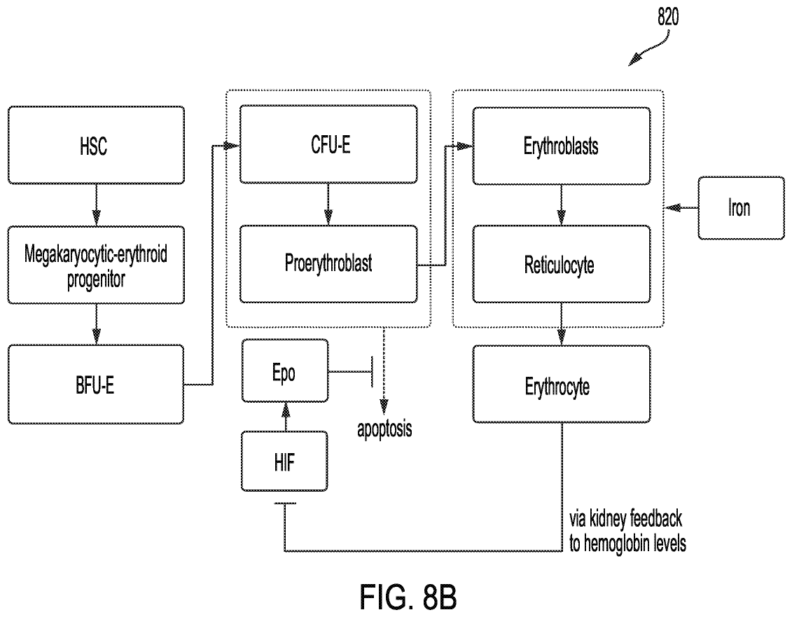

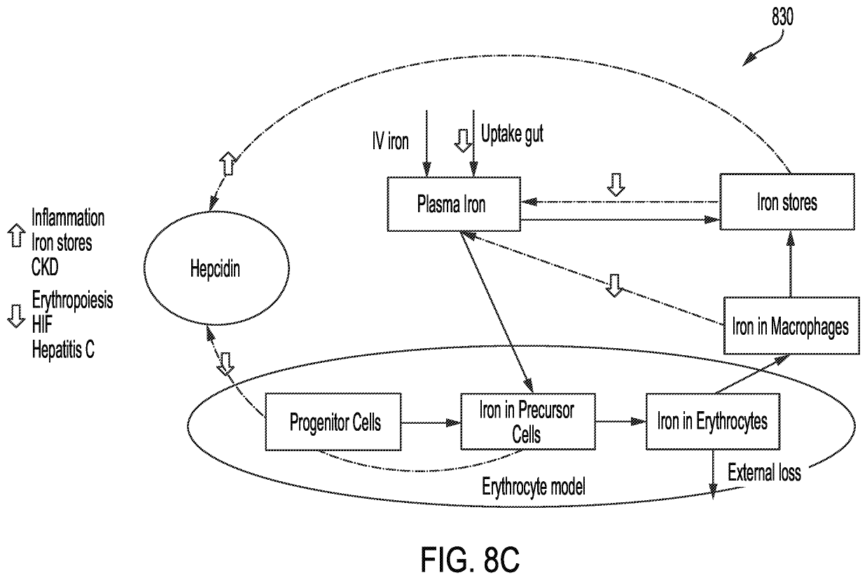

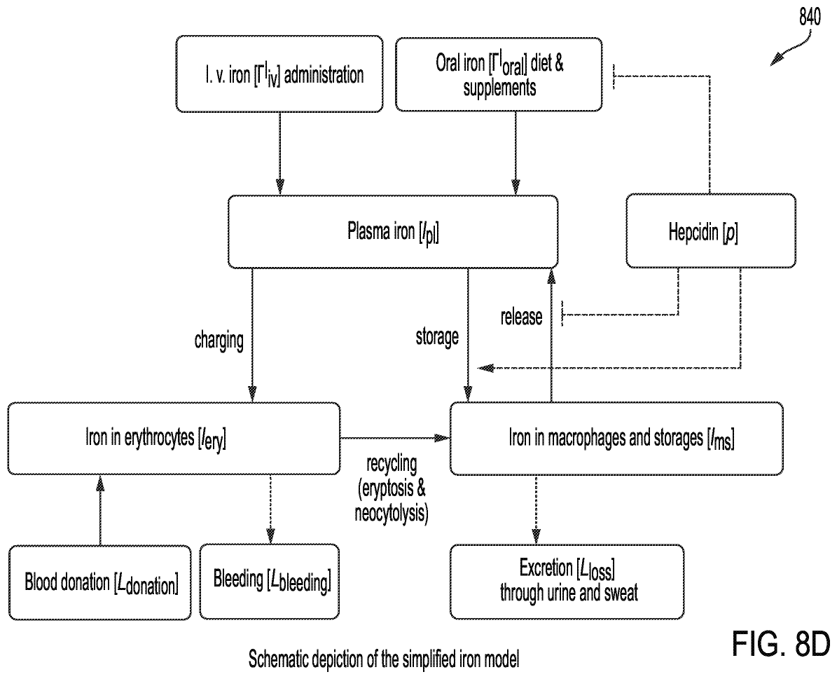

FIG. 8A shows a more detailed representation of FIG. 7. For instance, FIG. 8A includes various physiological processes at work such as clearance from serum, regulation of synthesis rates, degradation, cell fates, and so on.

Block 706 of the mathematical model 700 is the physiology of the HIF pathway and the erythropoietin (EPO). For instance, HIFs (hypoxia-inducible factors) are constitutively expressed transcription factors that elicit the response to decreased available oxygen levels by upregulating the expression of various target genes. The HIF transcriptional complex is a heterodimer of its subunits HIF-α and HIF-β. There are three isoforms of HIF-α termed HIF-1α, HIF-2α and HIF-3α. Under normoxic conditions, HIF-α is located in the cytoplasm; HIF-β is located in the nucleus. As O₂ concentration decreases, HIF-α (specifically, the isoform HIF-2α) is stabilized, translocates to the nucleus and binds to HIF-β to form the HIF transcriptional complex. The HIF complex regulates the transcription of target genes like EPO in kidney and liver. This is achieved by binding to hypoxia-responsive elements (HREs) in the respective gene promoters.

The HIF pathway includes multiple biochemical species such as HIF-α and HIF-β as well as the dimer that they form. To reduce model complexity, a single composite variable h is introduced that represents the “HIF signal”. More precisely, let n_{cmp} denote the amount of HIF transcriptional complexes bound to EPO promoter regions and n*_{cmp} denote its average in the absence of HIF-PHIs, then h can be defined by h(t)˜n_{cmp}(t)−n*_{cmp}.

Hence, the sign of h depends on whether n_{cmp} takes value above or below the steady state baseline level n*_{cmp}.

In healthy patients, the HIF signal is upregulated in the presence of hypoxia via the feedback in the kidneys. Here, a set point c*_{hgb} for the blood hemoglobin concentration is considered; the HIF signal reacts to any deviation from the set point. Moreover, since HIF-PHIs prevent HIF-α degradation, they effectively upregulate the amount of available HIF complexes that are able to bind EPO promoters. Therefore, h is upregulated by the presence of the HIF-PHI. These two contributions are combined in the following equations (1.6) and (1.7).

$$\frac{dh}{dt} = {β}_{h} ( θ ) [ {φ}_{{a}_{h} , {b}_{h} , {q}_{h , 0}} ( {k}_{h} [ {c}_{hgb} - {c}_{hgb}^{*} ] ) + {E}_{s} {ϕ}^{+} ( {[ \frac{s}{\overline{s}} ]}^{{m}_{h}} ) ] - {k}_{h} h , \qquad Eq . ( 1.6 )$$

$$where$$

$${c}_{hgb} = {λ}_{hgb} {n}_{ery} \qquad Eq . ( 1.7 )$$

c_{hgb} denotes the blood hemoglobin concentration as measured by the kidneys, given by the number of erythrocytes n_{ery} (a dynamic model variable, see Eq. (1.11)) and the conversion factor λ_{hgb}, which accounts for total blood volume and hemoglobin content per erythrocyte (see Eq. (1.24)). Furthermore, c*_{hgb} denotes the physiological hemoglobin set point and βₕ(θ) denotes the formation rate of HIF complexes. Functions of the type φ and ϕ^{±} are sigmoidal functions describing non-linear activation and inhibition. The functions φ and ϕ^{±} are defined as follows:

$${ϕ}^{+} ( x ) = \frac{x}{1 + x} \qquad Eq . ( A )$$

$${ϕ}^{-} ( x ) = \frac{1}{1 + x} \qquad Eq . ( B )$$

$${φ}_{a , b , q} ( x ) = a + \frac{b - a}{1 - {qe}^{- x}} \qquad Eq . ( C )$$

where x is an input variable and a, b and q are shape parameters determining the offset, height and steepness of the function. Eq. (A) and Eq. (B) may be referred to Hill type functions. Eq. (C) may be referred to as a Richards type equation.

Referring back to Eq. (1.6), the first term (φₐ_{ₕ}_{,b}_{ₕ}_{,q}_{_{h,0}}) describes the hypoxic upregulation of the HIF signal in response to decreases in the blood hemoglobin concentration. The second term

$$( {E}_{s} {ϕ}^{+} ( {[ \frac{s}{\overline{s}} ]}^{{m}_{h}} ) )$$

describes upregulation of the HIF signal in response to HIF-PHI administrations, where Eₛ is the HIF-PHI responsiveness, s̅ is the HIF-PHI EC50. EC50 is the half maximal effective concentration (e.g., the concentration at which half of the maximal effect is reached. mₕ is the hill coefficient, which describes the nonlinearity of the positive feedback of the HIF-PHI on the HIF signal. The parameter kₕ denotes the decay rate of the HIF signal. In other words, referring to Eq. (1.6), dh/dt indicates a rate of change of HIF signaling activity, βₕ(θ) indicates a formation rate of HIF complexes, φₐ_{ₕ}_{,b}_{ₕ}_{,q}_{_{h,0}} indicates a hypoxic upregulation of a HIF signal in response to decreases in blood hemoglobin concentration, kₕ indicates a decay rate of the HIF signal, c_{hgb} indicates a blood hemoglobin concentration measured by kidneys, c*_{hgb} indicates a physiological hemoglobin set point, Eₛ indicates a HIF-PHI responsiveness,

$${ϕ}^{+} ( {[ \frac{s}{\overline{s}} ]}^{{m}_{h}} )$$

indicates upregulation of the HIF signal in response to HIF-PHI administrations, and h indicates the HIF signal.

Serum EPO concentration is described by a second variable e, which is upregulated by the HIF signal and decays with a rate kₑ,

$$\frac{de}{dt} = {β}_{e} ( θ ) + {β}_{e} ( θ ) {φ}_{{a}_{e} , {b}_{e} , {q}_{e , 0}} ( {k}_{e} h ) - {k}_{e} e \qquad Eq . ( 1.8 )$$

The first term describes basal EPO synthesis under steady-state conditions. The second term describes additional EPO synthesis due to activation by the HIF signal. The third term describes EPO decay with rate kₑ. In other words, for Eq. (1.8), de/dt indicates a EPO concentration rate of change, βₑ(θ) indicates basal erythropoietin (EPO) synthesis under steady-state conditions, βₑ(θ)φₐ_{ₑ}_{,b}_{ₑ}_{,q}_{_{e,0}}(kₑh) indicates additional EPO synthesis due to activation by a HIF signal, and kₑe indicates EPO decay with a decay rate kₑ.

Blocks 708 and 712 of the mathematical model 700 are the physiology of red blood cell formation in the bone marrow and the blood hemoglobin concentration (clinical target variable). FIG. 8B is a schematic depiction of the erythropoietic lineage in the bone marrow and will be used to describe these blocks in more detail. In particular, red blood cells (erythrocytes) are continuously generated through the differentiation and proliferation of a hierarchy of stem and progenitor cells. Hematopoietic stem cells (HSCs) are at the apex of the hierarchy. They self-renew, while part of their progeny successively differentiates into more lineage-committed cell types (megakaryocytic-erythroid progenitors, erythroid burst-forming units [BFU-E], erythroid colony-forming units [CFU-E], proerythroblasts, erythroblasts and reticulocytes) as shown in FIG. 8B. This process is regulated through the HIF pathway, which in turn, regulates endogenous EPO production. EPO inhibits apoptosis (cell death) of progenitor cells. The final step (formation of red blood cells) also requires iron.

The erythropoietic lineage in the bone marrow consists of a multitude of different cell types and states. These cell types may be grouped according to whether their cell fates are regulated by EPO or iron, the master regulators in the mathematical model 700. As a result, the population sizes n_{prog}, nₚᵣₑ and n_{ery} of three composite cell populations are introduced that represent (i) progenitor populations (colony-forming units-erythroid (CFU-E) and proerythroblasts), (ii) immediate precursors of erythrocytes (erythroblasts and reticulocytes) and (iii) erythrocytes, respectively. To avoid huge numbers, all of these population sizes are normalized such that they denote the average progeny of a representative unit pool of BFU-Es (erythroid burst-forming unit).

In this scheme, apoptosis of the progenitor population is downregulated by EPO. Differentiation of precursors into erythrocytes is limited by iron availability; if not enough iron is available, cells that are primed for differentiation immediately die off, so that the total loss of precursors due to ‘attempted’ differentiation is constant. The corresponding dynamics is given by the following equations.

$$\frac{{dn}_{prog}}{dt} = {β}_{prog} ( θ ) - {J}_{prog \to pre} - {K}_{prog} , \qquad Eq . ( 1.9 )$$

$$\frac{{dn}_{pre}}{dt} = {2}^{{d}_{prog}} {J}_{prog \to pre} - {J}_{pre \to ery} - {K}_{pre} , \qquad Eq . ( 1.1 )$$

$$\frac{{dn}_{ery}}{dt} = {2}^{{d}_{pre}} {J}_{pre \to ery} - {K}_{ery} - {L}_{bleeding} + {L}_{donation} \qquad Eq . ( 1.11 )$$

Here, β_{prog}(θ) denotes the progenitor formation rate, which depends on patient specific demographic and physiological parameters θ, including weight, sex, etc. Thus, β_{prog} is proportional to the product of the number of available BFU-Es and the rate at which they differentiate. J_{i→j} denotes the differentiation flux from population i to j (e.g., population progenitor (prog) to precursors (pre) or pre to erythrocytes (ery)), Kᵢ (e.g., K_{prog} or Kₚᵣₑ) denotes the cell flux due to apoptosis, and the functions Lᵢ denote cell fluxes due to events external to erythropoiesis: L_{bleeding} and L_{donation} refer to bleeding events and blood donations, respectively. The factors of 2^{d}^{ᵢ} tindicate amplification due to an average number of d_{prog} or dₚᵣₑ rounds of cell division in the progenitor or precursor compartment, respectively. In other words, for Eq. (1.11), dn_{ery}/dt indicates a rate of change of the total amount of red blood cells, 2^{d}^{ₚᵣₑ} J_{pre→ery} indicates a differentiation flux from a precursors population to an erythrocytes population, K_{ery} indicates cell fluxes due to apoptosis, L_{bleeding} indicates cell fluxes due to bleeding events, and L_{donation} indicates cell fluxes due to blood donation events.

The total differentiation fluxes are given by the following equations.

$${J}_{prog} {\to }_{pre} = {ω}_{prog \to pre} {n}_{prog} , \qquad Eq . ( 1.12 )$$

$${J}_{pre \to ery} = {ω}_{pre \to ery} {v}_{mat} ( e ) {ϕ}^{+} ( {[ \frac{{ι}_{pl}}{{\overline{ι}}_{pl}} ]}^{{m}_{pre}} ) {n}_{pre} , \qquad Eq . ( 1.13 )$$

Here, ω_{i→j} (e.g., ω_{pre→ery} and ω_{prog}→ₚᵣₑ) denotes the differentiation rate from population i to j, lₚₗ denotes the total iron mass in plasma (a dynamic variable described further below) and l̅ₚₗ denotes the plasma iron EC50 for upregulating differentiation into erythrocytes. Furthermore, vₘₐₜ(e) denotes an epo-dependent regulation factor that leads to a de- or acceleration of maturation with changing Epo levels, which is described in the following equation.

vₘₐₜ(e)=φₐ_{ₘₐₜ}_{,b}_{ₘₐₜ}_{,q}_{_{mat,1}}(kₘₐₜ[e−e*ₘₐₜ]) Eq. (1.14)

where e*ₘₐₜ denotes Epo setpoint levels.

The cell death fluxes are given by the following equations.

$${K}_{prog} = {κ}_{prog} {ϕ}^{-} ( {[ \frac{e}{\overline{e}} ]}^{{m}_{prog}} ) {n}_{prog} , \qquad Eq . ( 1.15 )$$

$${K}_{pre} = {ω}_{pre \to ery} [ 1 - {ϕ}^{+} ( {[ \frac{{ι}_{pl}}{{\overline{ι}}_{pl}} ]}^{{m}_{pre}} ) ] {n}_{pre} , \qquad Eq . ( 1.16 )$$

$${K}_{ery} = {k}_{ery} {n}_{ery} + {k}_{neo} ( e ) {n}_{ery} , \qquad Eq . ( 1.17 )$$

where kᵢ (e.g., k_{ery} and K_{prog}) denote the apoptosis rates for cell population i and ē is the Epo EC50 for downregulation of progenitor apoptosis, kₙₑₒ denotes the rate of neocytolysis regulated by the hormone Epo given by:

kₙₑₒ(e)=φₐ_{ₙₑₒ}_{,b}_{ₙₑₒ}_{,q}_{ₙₑₒ}(kₙₑₒ[e−eₙₑₒ]), Eq. (1.18)

where eₙₑₒ denotes the threshold for neocytolysis.

Note that the amount of available iron determines the relative fraction of precursor cells that are primed for apoptosis or differentiation, respectively; however, the overall ‘loss’ from the precursor cell compartment is independent of iron and given by J_{pre→ery}+Kₚᵣₑ=ω_{pre→ery}*nₚᵣₑ.

Loss and gain terms describing bleedings and blood donations are of the form,

$${L}_{bleeding} = {\sum }_{i} δ ( t - {t}_{i} ) \frac{{n}_{ery} ( {t}_{i} )}{{V}_{blood} ( {t}_{i} )} Δ {V}_{i} \qquad Eq . ( 1.19 )$$

$${L}_{donation} = {\sum }_{i} δ ( t - {t}_{i} ) Δ {n}_{ery , i} \qquad Eq . ( 1.2 )$$

Here, the sums run over all bleedings and blood donations, respectively, and ΔVᵢ denotes the volume of blood lost or donated. V_{blood}(ti) is the total blood volume at the time tᵢ of loss/gain. In the case of donations, n_{ery,i} denotes the average number or erythrocytes per blood bottle. δ is the Dirac delta function, which is an idealized mathematical construct used to represent instantaneous changes in a variable (e.g., the quasi-instantaneous change in erythrocyte numbers due to bleedings or blood donations).

If explicit iron dynamics is not considered (assumption of sufficient iron availability for all dynamical regimes), the iron regulatory factor is considered to be constant, which leads to the following equation.

$${ϕ}^{+} [ \frac{{ι}_{pl}}{{\overline{ι}}_{pl}} ] \to {ϕ}_{iron} \qquad Eq . ( 1.21 )$$

which implies that, for self-consistency reasons, l must be chosen such that

$${ϕ}^{+} [ \frac{{ι}_{pl}^{*}}{{ι}_{pl}} ] = {ϕ}_{iron} \qquad Eq . ( 1.22 )$$

is fulfilled, where ι̅ₚₗ denotes the steady state of ιₚₗ if the iron module is turned on.

Regarding the hemoglobin and observables, since the variables nᵢ denote the average progeny of a representative pool of BFU-Es (erythroid burst-forming unit), absolute cell population sizes are given by Ni=Q_{prog}nᵢ, where Q_{prog}=10⁹ is the conversion factor. Accordingly, the blood hemoglobin concentration is given by

$${c}_{hgb} = {λ}_{hgb} {n}_{ery} \qquad Eq . ( 1.7 )$$

$$where$$

$${λ}_{hgb} = \frac{MCH}{{V}_{blood}} {Q}_{prog} \qquad Eq . ( 1.23 )$$

In typical units, this reads:

$${{λ}_{hgb} [ g / dl ]}^{- 1} = {10}^{- 1} * \frac{{MCH [ pg ]}^{- 1}}{{{V}_{blood} [ ml ]}^{- 1}} \qquad Eq . ( 1.24 )$$

Block 710 of the mathematical model 700 is the physiology of iron balance. Regarding the biology, 4 iron pools are considered within the body: a plasma pool, iron in precursor cells, iron in erythrocytes and a pool describing iron in the macrophages and stores. Hepcidin regulates iron homeostasis (rapid regulation and rapid clearance in plasma). Increased erythropoiesis, increased HIF levels and depleted stores decrease the amount of hepcidin released into plasma. Large iron stores and inflammation increase hepcidin levels. Iron is lost via loss of cells (red blood cells).

Iron is a part of hemoglobin which provides the means of O2 transport to the tissue. The rate at which iron can be released to plasma from existing stores and macrophages may easily limit the rate of iron delivery for hemoglobin synthesis. Therefore, a strong relationship between impaired erythropoiesis and total body iron burden exists. Iron is a substance which is very tightly controlled from the body as iron overload is toxic. Iron homeostasis can be only achieved by the control of absorption because the human body, in contrast to other mammals, is not able to influence the excretion of iron. Under normal circumstances the average daily loss of iron is very small (Male :˜1 mg, Female :˜2 mg, because of menstruation). However, in hemodialysis daily iron need is around 5-7 mg or even higher. This is above the normal absorptive capacity from diet. Consequently, 80-90% of patients on rHuEPO require iron supplementation at some stage in their therapy.

For the iron submodel, five dynamic iron pools are considered within the body: a plasma pool (called “available iron”), iron in precursor cells, iron in erythrocytes, iron in the macrophages and a storage pool (ferritin and hemosiderin stores are lumped together). Other iron in the body is ignored. FIG. 8C below shows the organizational structure of the iron sub-model. The available iron pool refers to transferrin bound iron in plasma only and we do not distinguish between monoferric and diferric transferrin. Further, free iron is ignored. Hence, the assumption is that the amount of iron in plasma is proportional to the amount of transferrin molecules carrying iron. Hepcidin is the main regulator of iron homeostasis and its effect is exerted through a change in ferroportin, a transmembrane protein that transports iron from the inside of a cell to the outside of the cell. Ferroportin is the only known iron exporter and thus is crucial for the uptake and redistribution of iron in the body. Note, the effects of changes in hepcidin levels can last longer than the change in the substance in plasma as the main effect is through a change in ferroportin. Hepcidin is rapidly upregulated in the case of inflammation or infection. It also increases in the case of large iron stores. However, the effect of an increased erythropoiesis outweighs the effect of large iron stores. In chronic kidney disease (CKD) patients, hepcidin levels are in general increased, independent of inflammation (hepcidin is partly cleared by the kidneys). Further, decreased hepcidin levels are observed in patients suffering from Hepatitis C. Elevated hepcidin levels are sufficient to cause anemia and hypoferremia. The amount of iron lost via urine and sweat is neglected. Thus, in the model 700, excretion of iron takes only place when cells are lost. This can be due to loss of RBCs, i.e. external bleeding, loss of red cells in the dialyzer, blood draws. Further, there is a daily loss of epithelial cells, which amount for about 1 mg/day. Note, this is not modeled directly but is effectively lost from the erythrocyte compartment. An assumption is made that only the precursor cells in the bone marrow uptake iron and ineffective erythropoiesis depends on the amount of iron which can be provided for erythropoiesis. All circulating red blood cells carry an average corpuscular hemoglobin content throughout their lifespan. Loss of iron from erythrocytes during senescence is not accounted for in the model 700.

Regarding the model for iron and in particular, the hepcidin feedback, the regulator of iron homeostasis is hepcidin, a peptide hormone produced by the liver. Hepcidin (hepatic bactericidal protein) negatively regulates the availability of iron in plasma by direct inhibition of ferroportin. This is the iron exporter required for iron egress from iron exporting cells, as for instance, enterocytes and macrophages. It acts as a medium to withhold iron from certain invasive bacteria, as they are not able to proliferate under conditions of insufficient supply of iron. Hepcidin is synthesized and secreted by the liver, catabolized by ferroportin expressing cells and partly excreted by the kidneys. The simplest description of this dynamic is of the form:

$$\frac{dp}{dt} = {β}_{p} ( θ ) [ 1 + {φ}_{{a}_{p} , {b}_{p} , {q}_{p , 0}} ( x - {x}^{*} ) ] - {κ}_{p} p \qquad Eq . ( 1.25 )$$

Here, p(t) describes the level of hepcidin in plasma, βₚ(θ) is the concentration of secreted hepcidin at baseline, the function φ describes the up- and downregulation of hepcidin secretion due to various factors x with baseline values x* and κₚ denotes the degradation (catabolism and excretion) of hepcidin. Note, hepcidin is upregulated rapidly and degrades very quickly. Thus, the dynamics of the hepcidin concentration in plasma can be calculated using an equilibrium assumption:

$$p = \frac{{β}_{p} ( θ )}{{κ}_{p}} [ 1 + {φ}_{{a}_{p} , {b}_{p} , {q}_{p , 0}} ( x - {x}^{*} ) ] \qquad Eq . ( 1.26 )$$

The production of hepcidin beyond baseline value is obtained as the result of a feedback loop involving the precursor cell population nₚᵣₑ, the effective HIF concentration h, the amount of stored iron lₘₛ and the level of inflammation of the patient. The latter two effects can be lumped together and serum ferritin levels c_{fer} may be used as a proxy to describe the influence of the two processes. The Richards' curve function argument x−x* can be written as

$$x - {x}^{*} = {α}_{pre} ( \frac{{n}_{pre}^{*}}{{n}_{pre}} - 1 ) + {α}_{pre} ( \frac{{c}_{fer}}{{c}_{fer}^{*}} - 1 ) - {α}_{h} h \qquad Eq . ( 1.27 )$$

Moreover, the secretion rate is downregulated in patients suffering from Hepatitis C, which can be realized by changing the baseline production βₚ to βₚ^{HepC}. Further, the degradation rate kp is lowered in patients with impaired kidney function due to a decreased clearance of hepcidin by the kidneys:

κₚ=f(m_{rrf}) Eq. (1.28)

Here m_{rrf} refers to the residual renal function of the patient. For patients on hemodialysis, who rapidly loose any remaining residual renal function after initiating this maintenance treatment modality, this can be simplified to

κₚ=κₚ^{HD} Eq. (1.29)

where κₚ^{HD} is a constant degradation rate reflecting the metabolization of hepcidin within the body.

Regarding iron update, two types of iron update is considered: oral and intravenous (i.v.). Oral uptake of iron is described through an intermediate gastrointestinal (GI) compartment. The dynamics of the iron mass \tilde{ι} in the GI tract is given by:

$$\frac{d \tilde{ι}}{dt} = {Γ}_{oral}^{ι} ( t ) - {\tilde{α}}_{ι} ( p ) \tilde{ι} - {\tilde{k}}_{ι} \tilde{ι} , \qquad Eq . ( 1.3 )$$

Here, \tilde{α}_{ι}(p) denotes the Hepcidin-dependent absorption rate and \tilde{k}_{ι} denotes the clearance rate in the GI tract. If the absorption rate \tilde{α}_{ι} is parameterized as:

$${\tilde{α}}_{ι} ( p ) = \frac{{\tilde{k}}_{ι}}{{{f}_{ι} ( p )}^{- 1} - 1} , {f}_{ι} ( p ) - {ω}_{ι} {ϕ}^{-} ( {[ \frac{p}{{\tilde{p}}_{gi}} ]}^{{m}_{gi}} ) , \qquad Eq . ( 1.31 )$$

where ωι is a constant factor, the bioavailability of iron is given by fι, which is described further in Eq. (1.5) above. In the limit \tilde{k}_{ι}→∞, the GI iron mass equilibrates infinitely fast, leading to an instantaneous absorption rate of \tilde{α}_{ι}(p)\tilde{ι}→f_{ι}(p)Γₒᵣₐₗ^{ι}(t) while the mass resident in the GI tract tends to zero \tilde{ι}→0.

I.V. iron administrations enter directly into the plasma compartment, which is described below.

FIG. 8C shows the organizational diagram of the iron sub-model. For instance, FIG. 8C depicts the main iron compartments within the body. The red cell lineage are shown by the progenitor, the precursor and the erythroid cells—named “erythrocyte model”. The plasma iron, iron stores, iron in macrophages, iron in precursor cells, and iron in erythrocytes denote blocks with iron. The progenitor cells are not denoted with iron, as these type of cells only carry an insignificant amount of iron. The plasma iron block refers to iron circulating in the plasma. Hepcidin is the main regulator of iron homeostasis and determines to a large extent the availability of iron for red cell production. Transfer rates that are influenced by hepcidin include iron stores to hepcidin, iron in precursor cells to hepcidin, iron in macrophages to plasma iron, and iron stores to plasma iron; constant rates include plasma iron to iron stores, iron in macrophages to iron stores, iron in erythrocytes to iron in macrophages, external loss, plasma iron to iron in precursor cells, progenitor cells to iron in precursor cells, and iron in erythrocytes. The up and down arrows depict a down-or upregulation of the transfer with increasing hepcidin levels. The arrows and labels next to the “hepcidin box” indicate whether a condition leads to an up- or downregulation of hepcidin.

The full model for iron exchange between physiological compartments is described below. The general form of the compartmental iron model is as follows:

$$\frac{d {ι}_{pl}}{dt} = {Γ}_{iv}^{ι} ( t ) + {\tilde{α}}_{ι} ( p ) \tilde{ι} + {J}_{ms \to pl}^{ι} - {J}_{pl \to ms}^{ι} - {J}_{pl \to ery}^{ι} , \qquad Eq . ( 1.32 )$$

$$\frac{d {ι}_{pre}}{dt} = {J}_{pl \to pre}^{ι} - {J}_{pre \to ery}^{ι} ,$$

$$\frac{d {ι}_{ery}}{dt} = {J}_{pl \to ery}^{ι} - {J}_{ery \to ms}^{ι} - {L}_{bleeding} {Q}_{ery}^{ι} + {L}_{transfusion} {Q}_{ery}^{ι}$$

$$\frac{d {ι}_{ms}}{dt} = {J}_{ery \to ms}^{ι} - {J}_{ms \to pl}^{ι} + {J}_{pl \to ms}^{ι} - {L}_{loss}^{ι} ,$$

Note, in the above equations, the dependence of particular components on t was not explicitly stated to simplify notation. Furthermore, ι denotes the amount of iron in the different compartments and J^{ι} describes the fluxes of iron from red cell compartments to other compartments. Further, administration of iron is denoted by Γᵢᵥ^{ι} (intravenously injected iron, oral supplementations and iron in diet). The terms Lι describe loss of iron that occurs unrelated to loss of red cells and gain of iron due to blood transfusion, respectively. Since the time needed to administer iron intravenously is very short compared to other time constants in the system, administrations as series of Dirac delta can be described as,

Γᵢᵥ^{ι}(t)=Σᵢιᵢδ_{σ}(t−tᵢ) Eq. (1.33)

Here, ιᵢ are the uptaken iron masses, Γ_{diet}^{ι} is a constant influx of iron through the diet (a system parameter), Γᵢᵥ^{ι}(t) refers to the iron administered to the patient intravenously, and

$${δ}_{σ} ( t ) = t - {t}_{i} {e}^{\frac{- {t}^{2}}{2 {σ}^{2}}} / ( \sqrt{2 π} σ )$$

is a normalized Gaussian serving as a regularized delta pulse with width σ.

FIG. 8D is similar to FIG. 8C and describes how iron is lost from the system (e.g., via bleeding, execration) and gained in case of blood transfusion (e.g., blood donation in FIG. 8).

A condensed model for iron exchange between physiological compartments is described below. Considering the model structure of the erythrocyte model described above, the equations stated in Eq. (1.32) can be further simplified. Iron is synthesized into hemoglobin by the erythroid precursor cell population and recycled when mature erythroid cells are phagocytosed. Further, iron is lost from the body when red blood cells are lost (bleeding, blood draws, dialyzer, etc). However, because of the structure of the erythroid cell compartment the iron flux and dynamics in the erythroid compartments does not need to be described explicitly but can effectively be linked to the differentiation flux Jpre→ery, the eryptosis and neocytolysis flux Kery and loss functions Li of erythroid cells. Thus Eq. (1.32) simplifies to:

$$\frac{d {ι}_{pl}}{dt} = {Γ}_{iv}^{ι} ( t ) + {\tilde{α}}_{ι} ( p ) \tilde{ι} + {J}_{ms \to pl}^{ι} - {J}_{pl \to ms}^{ι} - {J}_{pl \to ery}^{ι} , \qquad Eq . ( 1.34 )$$

$$\frac{d {ι}_{ms}}{dt} = {J}_{ery \to ms}^{ι} - {J}_{ms \to pl}^{ι} + {J}_{pl \to ms}^{ι} - {L}_{loss}^{ι} ,$$

Here, p denotes Hepcidin levels and Lₗₒₛₛ^{ι} is an effective loss term accounting for iron excretion loss through urine and sweat. The iron fluxes between compartments are given by:

$${J}_{ms \to pl}^{ι} = {ϕ}^{-} ( {[ \frac{p}{{\overline{p}}_{ms}} ]}^{{m}_{ms}} ) {ω}_{ms \to {pl}^{ι} ms} \qquad Eq . ( 1.35 )$$

$${J}_{pl \to ms}^{ι} = {ϕ}^{+} ( {[ \frac{{ι}_{pl}}{{\overline{ι}}_{pl , 2}} ]}^{{m}_{pl}} ) {ω}_{pl \to {ms}^{ι} pl}$$

$${J}_{pl \to ery}^{ι} = {2}^{{d}_{pre}} {J}_{pre \to ery} {Q}_{ery}^{ι} ,$$

$${J}_{ery \to ms}^{ι} = {K}_{ery} {Q}_{ery}^{ι} ,$$

J_{pre→ery}, K_{ery}, kₙₑₒ and L_{bleeding} are defined in Eqs. (1.11) and (1.17). Here, Q_{ery}^{ι} denotes the iron content per erythrocyte.

For reference, the entrained dynamics of the erythrocytic iron ι_{ery}=n_{ery}Q_{ery}^{ι} are noted as:

$$\frac{d {ι}_{ery}}{dt} = {J}_{pl \to ery}^{ι} - {J}_{ery \to ms}^{ι} - {L}_{bleeding} {Q}_{ery}^{ι} + {L}_{transfusion} {Q}_{ery}^{ι} .$$

The iron content per erythrocyte can be calculated as:

Q_{ery}^{ι}=(Q_{prog}Q_{hgb}MCH)/(Q_{|g|→|pg|}) Eq. (1.36)

Ferritin can be computed self-consistently as:

$${c}_{fer} ( t ) = \frac{{ι}_{ms}}{{ι}_{ms}^{*} / {c}_{fer}^{*}} \qquad Eq . ( 1.37 )$$

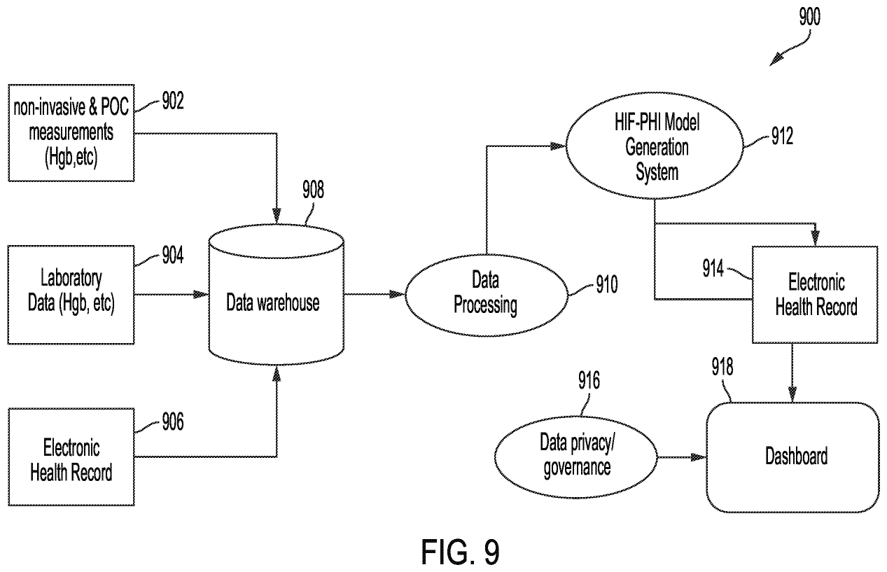

FIG. 9 is a data flowchart of an exemplary process for obtaining and using the population patient data according to one or more examples of the present application. For instance, a data warehouse 908 may be a storage entity (e.g., a server and/or other type of computing apparatus that includes memory for storing information). The data warehouse 908 may receive information for the patients from a plurality of sources. For instance, the data warehouse 908 may receive non-invasive and point of care (POC) measurements 902 such as HGB and/or other measurements. The data warehouse 908 may further receive laboratory data 904 (e.g., HGB and/or so on) as well as electronic health records 906 (e.g., information for patients from an electronic health record).

Afterwards, data processing 910 may be performed for the information stored in the data warehouse 908. For instance, data processing 910 may be performed so as to standardize the information within the data warehouse 908 prior to the HIF-PHI model generation system 912 using the population patient data. For example, the data warehouse 908 may receive data (e.g., non-invasive measurements and/or laboratory data) in many different data formats and from many different sources. In order to use the data for determining a plurality of virtual patient avatars and/or a set of individualized model parameters for a patient, the different data formats may need to be standardized. As such, the data processing 910 standardizes (e.g., converts) the data from the data warehouse 908 into a standardized data format. Subsequently, the HIF-PHI model generation system 912 may use the standardized data format (e.g., the population patient data) as described above. For instance, the HIF-PHI model generation system 912 may use the standardized data for determining (e.g., developing and/or updating) an anemia protocol using a large cohort of HIF avatars, and/or by conducting a large number of virtual clinical trials. Additionally, and/or alternatively, the HIF-PHI model generation system 912 may include anemia software (e.g., instructions stored in memory) that when executed by a processor, determines personalized HIF avatars (e.g., personalized or individualized HIF-PHI models), and updates these personalized HIF avatars regularly with patient-specific data so as to provide optimal treatment recommendations for patients.

Furthermore, the HIF-PHI model generation system 912 may provide information to the electronic health record 914. This information may in turn be provided to a dashboard 918 (e.g., a medical dashboard that displays a next recommended HIF-PHI dosage such as a medical dashboard of the HIF-PHI administration computing device 104 shown and described in FIG. 2). Furthermore, data privacy/governance 916 may also be used to ensure the data displayed by the dashboard 918 is compliant.

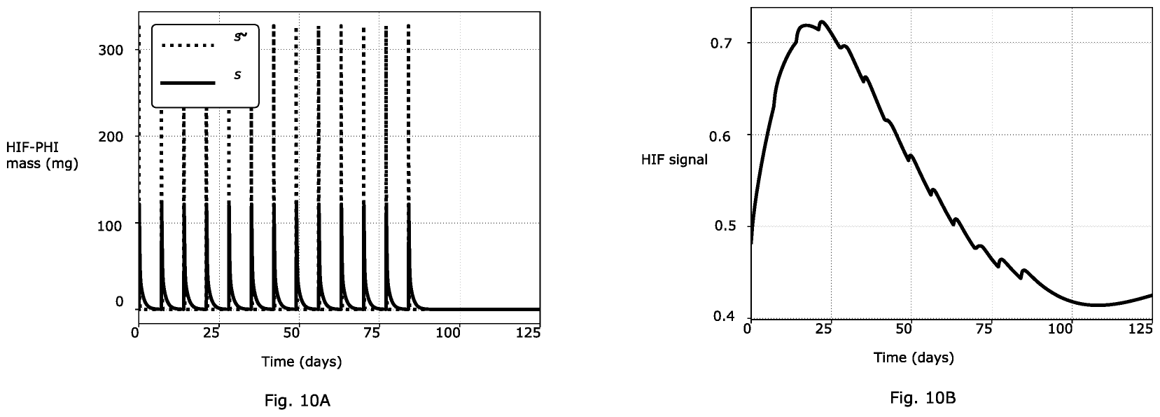

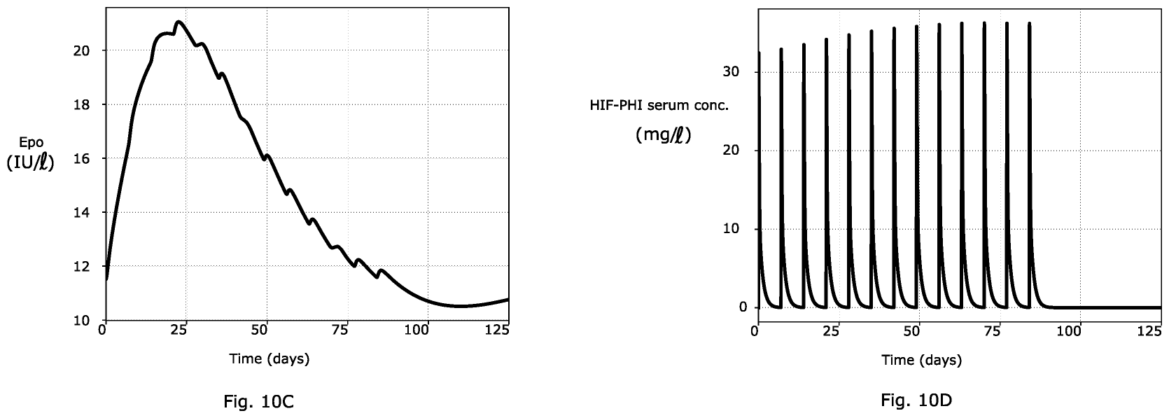

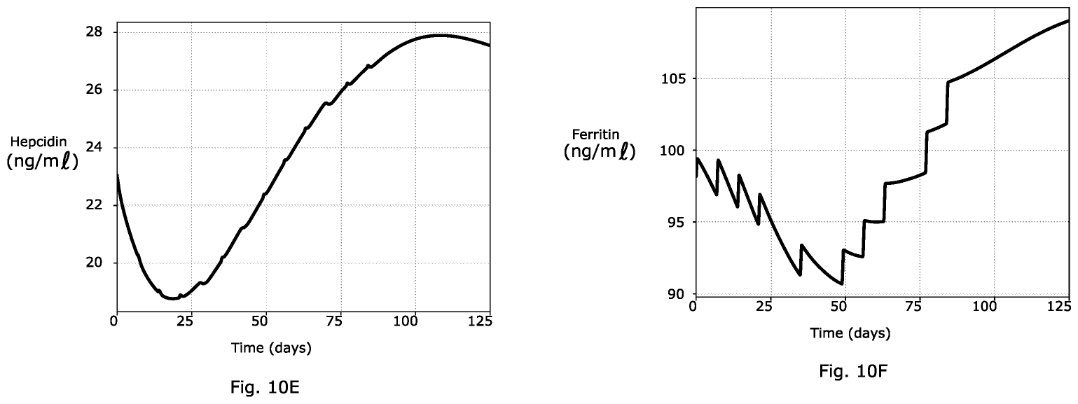

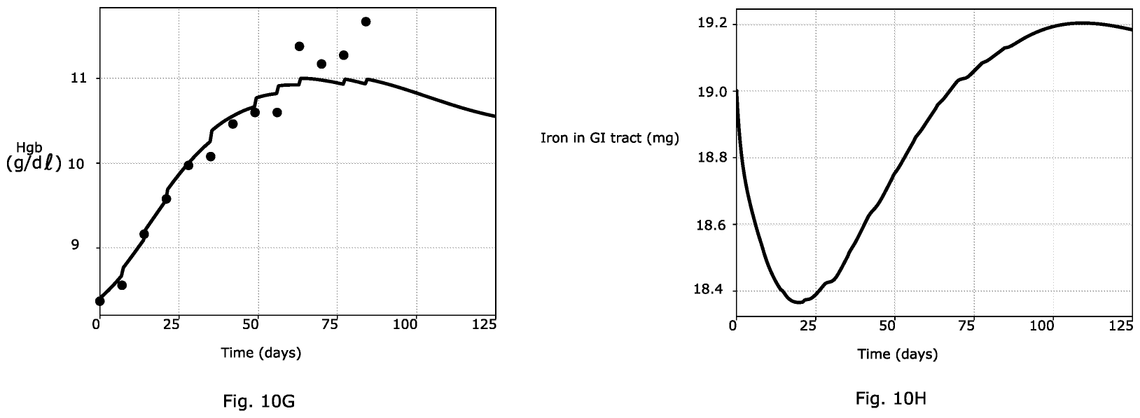

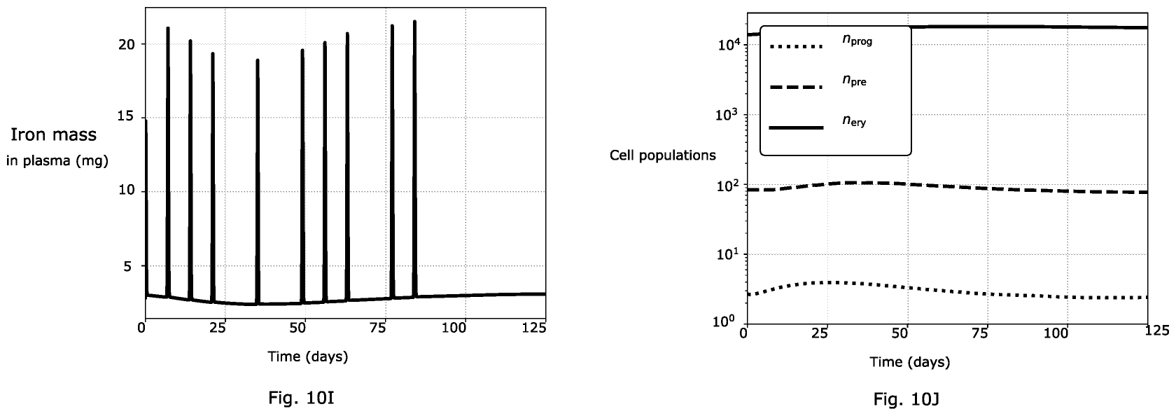

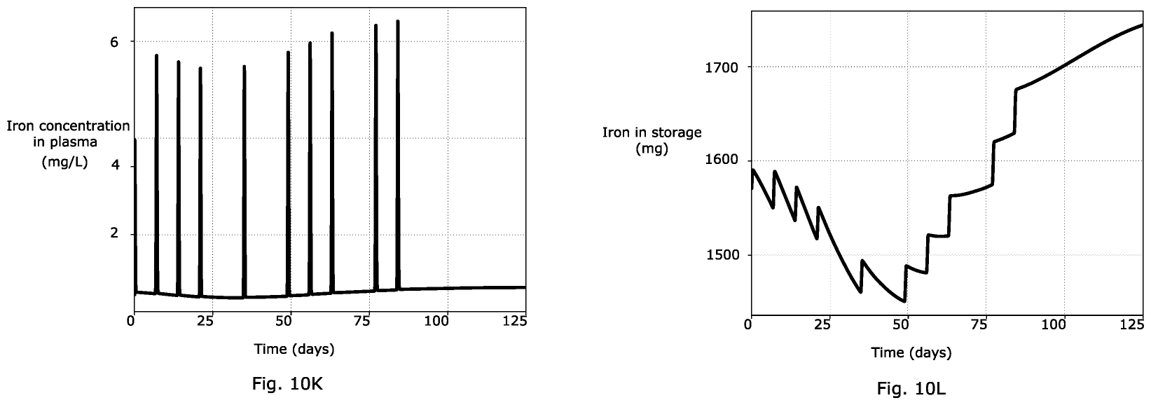

As will be described below, FIGS. 10A-17L show graphical representations for modeling the HIF-PHI administrations using the HIF-PHI model and the mathematical model described above. In particular, FIGS. 10A-17L show output results (e.g., the personalized model parameters/variables described above) over a period of time, and for a specific set of patients. The data that is input into the models to generate the graphical representations shown in FIGS. 10A-17L is from the sources provided below.

FIGS. 10A-10L show graphical representations for modeling HIF-PHI administrations according to one or more examples of the present application. For instance, as mentioned previously, obtained patient data may be input into the mathematical model described above. Based on inputting the patient data into the mathematical model, output results may be determined. The graphical representations of FIGS. 10A-10L show the output results (e.g., the personalized model parameters/variables described above) over a period of time, and for a specific set of patients (e.g., patients that took a series of HIF-PHI ROXADUSTAT administrations with concomitant intravenous iron administrations). For instance, FIG. 10A shows the HIF-PHI mass (model variables \tilde{s} and s from Eqs. 1.1 and 1.2-1.4, respectively) over a period of time. FIG. 10B shows the HIF signal (model variable h, Eq. 1.6) over a period of time. FIG. 10C shows the EPO mass (model variable e, Eq. 1.8) over a period of time. FIG. 10D shows the HIF-PHI serum concentration (model variable s, Eq. 1.1, divided by the plasma volume) over a period of time. FIG. 10E shows the Hepcidin (model variable p, Eq. 1.26) over a period of time. FIG. 10F shows the Ferritin (model variable

$${c}_{fer} = \frac{{ι}_{ms}}{{ι}_{ms}^{*} / {c}_{fer}^{*}} ,$$

Eq. 1.32) over a period of time. FIG. 10G shows the hemoglobin (model variable c_{hgb}, Eq. 1.7) over a period of time as well as shows the actual measured HGB data points (e.g., the dots). The data points are digitized from FIG. 2 in Besarab et al., “Roxadustat (FG-4592): Correction of Anemia in Incident Dialysis Patients”, J. Am. Soc. Nephrol. 27, 1225-1233 (2016). In other words, FIG. 10G shows a curve denoting the calculated HGB based on inputting the patient data into the mathematical model as well as data points denoting the actual measured HGB from the patient data. FIG. 10H shows the iron mass in GI tract (model variable \tilde{ι}, Eq. 1.30) over a period of time. FIG. 10I shows the iron mass in plasma (model variable ιₚₗ, Eq. 1.32) over a period of time. FIG. 10J shows the abundance of cell populations (model variables n_{prog}, nₚᵣₑ, n_{ery}, Eqs. 1.9-1.11) over a period of time. FIG. 10K shows the iron concentration in plasma (model variable ιₚₗ, Eq. 1.32, divided by the plasma volume) over a period of time. FIG. 10L shows the iron mass in storage (model variable ιₘₛ, Eq. 1.32) over a period of time.

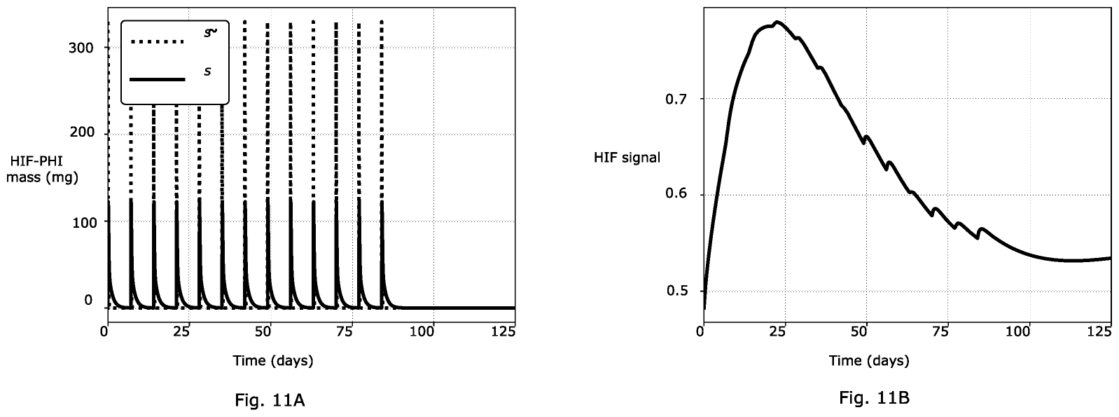

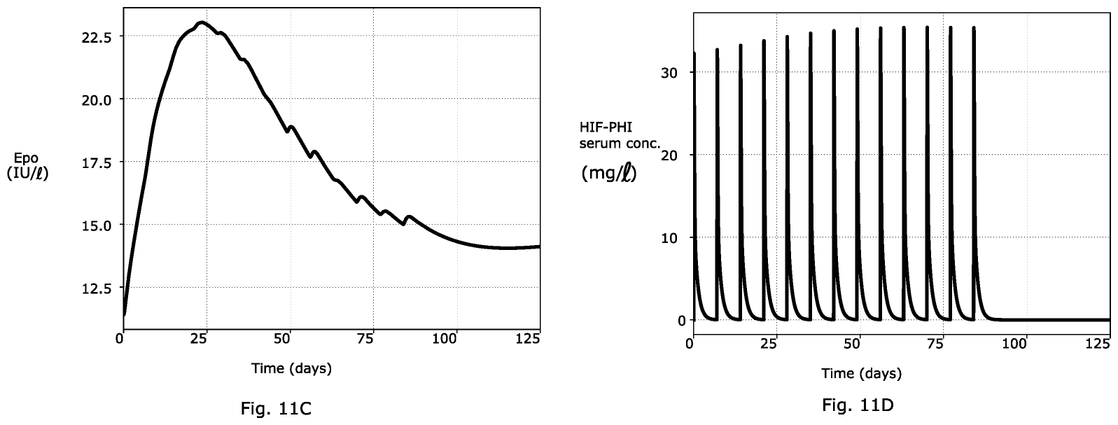

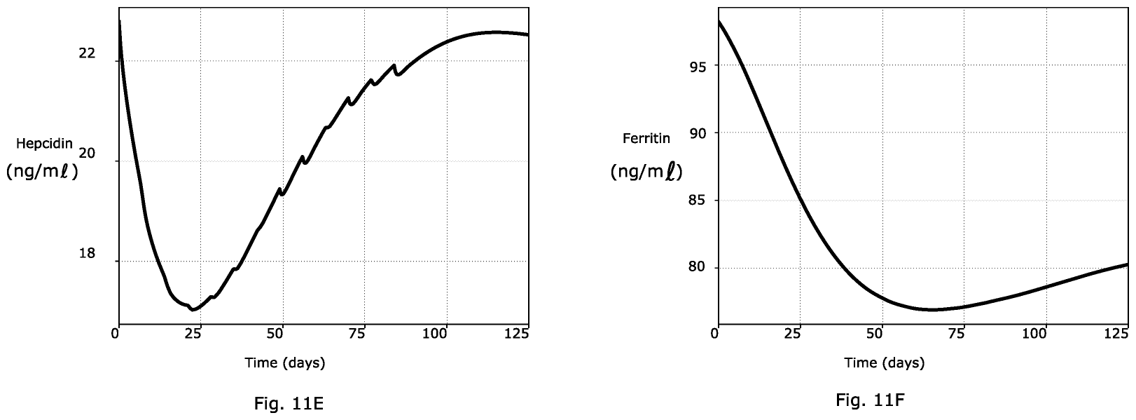

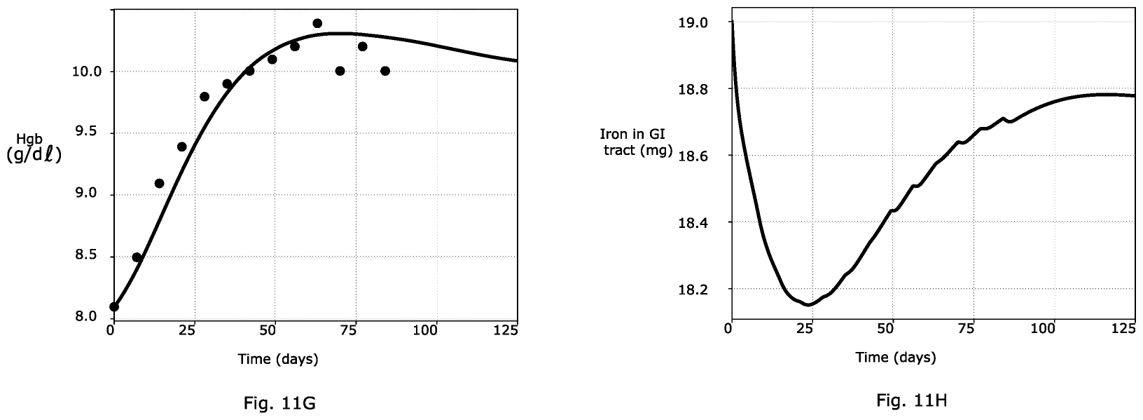

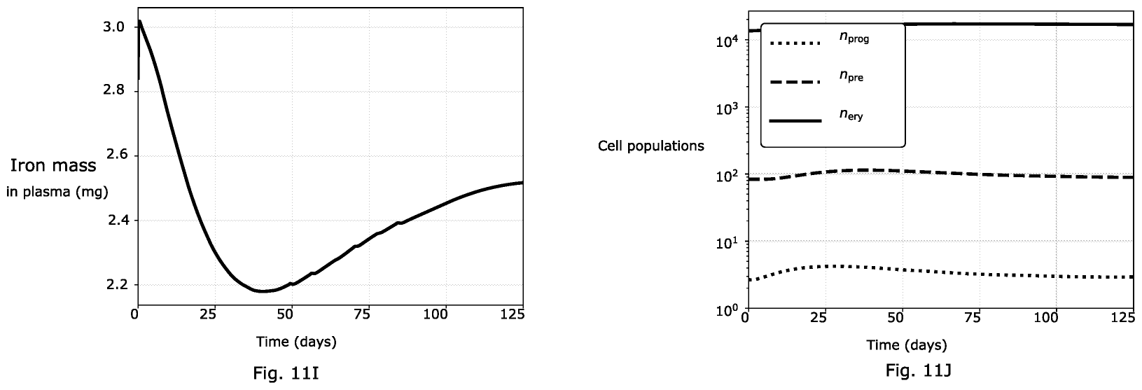

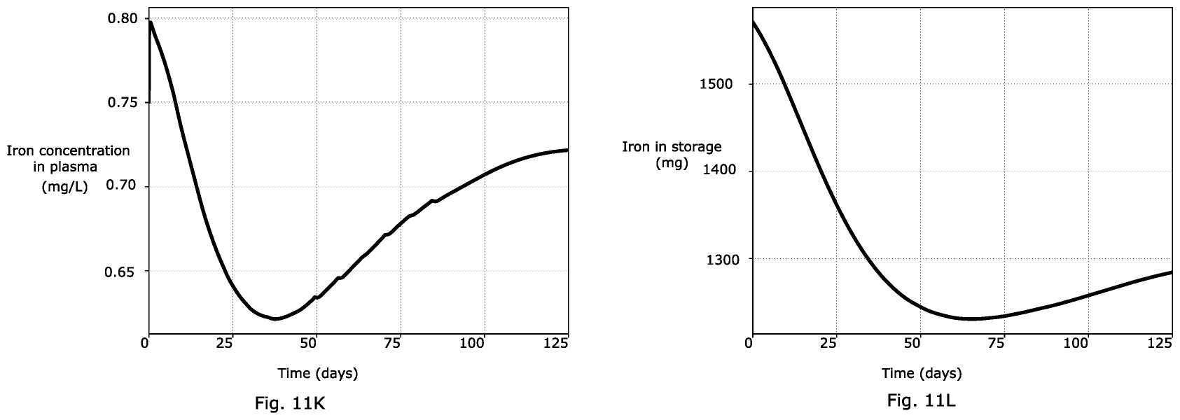

FIGS. 11A-11L show graphical representations for modeling HIF-PHI administrations according to one or more examples of the present application. The graphical representations of FIGS. 11A-11L show the output results (e.g., the personalized model parameters/variables described above) over a period of time, and for a specific set of patients (e.g., patients that took a series of HIF-PHI ROXADUSTAT administrations with no concomitant iron administrations). For instance, FIG. 11A shows the HIF-PHI mass (model variables \tilde{s} and s from Eqs. 1.1 and 1.2-1.4, respectively) over a period of time. FIG. 11B shows the HIF signal (model variable h, Eq. 1.6) over a period of time. FIG. 11C shows the EPO mass (model variable e, Eq. 1.8) over a period of time. FIG. 11D shows the HIF-PHI serum concentration (model variable s, Eq. 1.1, divided by the plasma volume) over a period of time. FIG. 11E shows the Hepcidin (model variable p, Eq. 1.26) over a period of time. FIG. 11F shows the Ferritin (model variable

$${c}_{fer} = \frac{{ι}_{ms}}{{ι}_{ms}^{*} / {c}_{fer}^{*}} ,$$

Eq. 1.32) over a period of time. FIG. 11G shows the hemoglobin (model variable c_{hgb}, Eq. 1.7) over a period of time as well as shows the actual measured HGB data points (e.g., the dots). The data points are digitized from FIG. 2 in Besarab et al., “Roxadustat (FG-4592): Correction of Anemia in Incident Dialysis Patients”, J. Am. Soc. Nephrol. 27, 1225-1233 (2016). In other words, FIG. 11G shows a curve denoting the calculated HGB based on inputting the patient data into the mathematical model as well as data points denoting the actual measured HGB from the patient data. FIG. 11H shows the iron mass in GI tract (model variable \tilde{ι}, Eq. 1.30) over a period of time. FIG. 11I shows the iron mass in plasma (model variable ιₚₗ, Eq. 1.32) over a period of time. FIG. 11J shows the abundance of cell populations (model variables n_{prog}, nₚᵣₑ, n_{ery}, Eqs. 1.9-1.11) over a period of time. FIG. 11K shows the iron concentration in plasma (model variable ιₚₗ, Eq. 1.32, divided by the plasma volume) over a period of time. FIG. 11L shows the iron mass in storage (model variable ιₘₛ, Eq. 1.32) over a period of time.

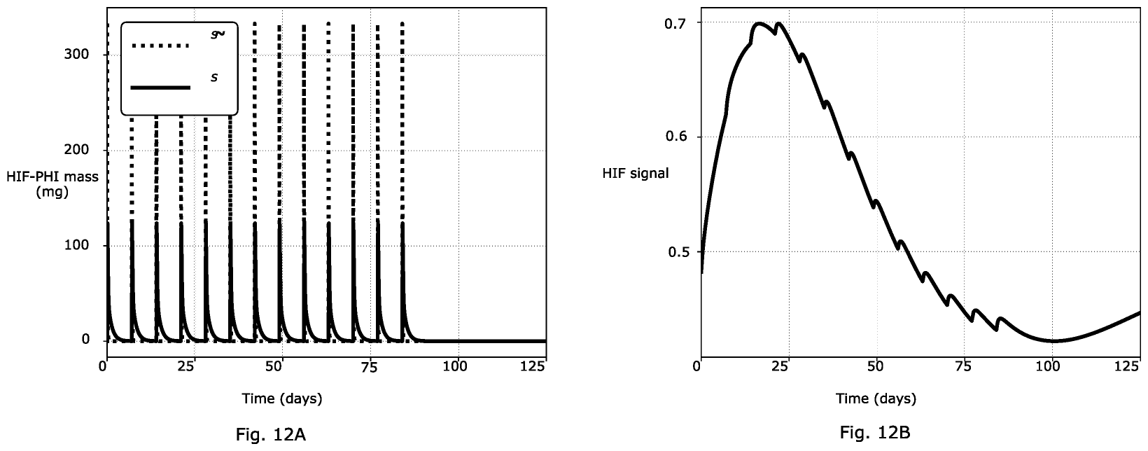

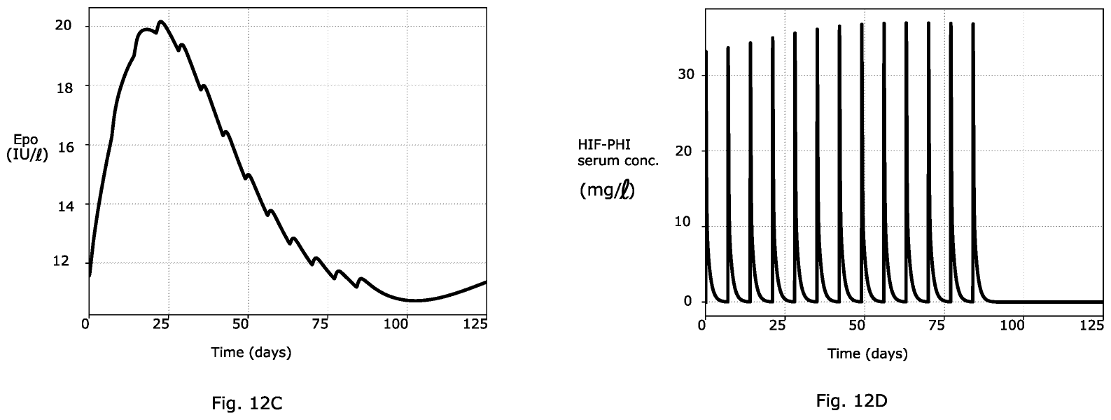

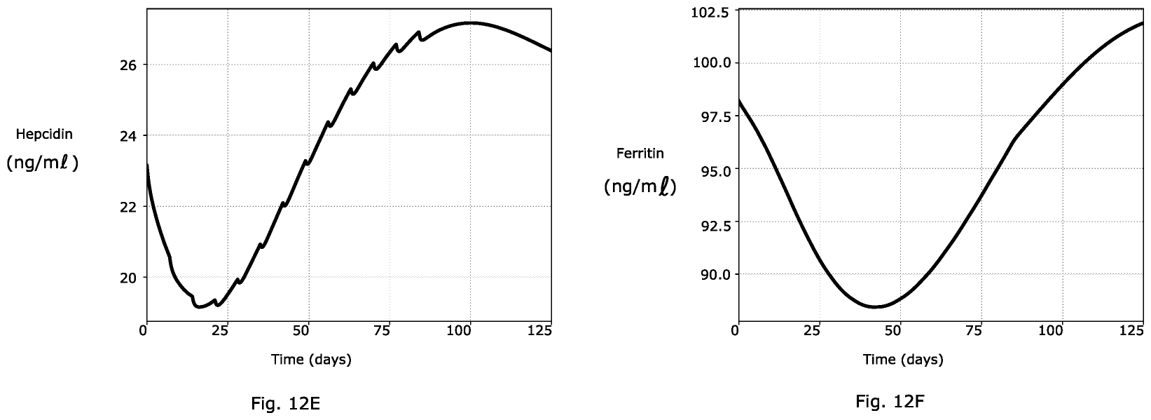

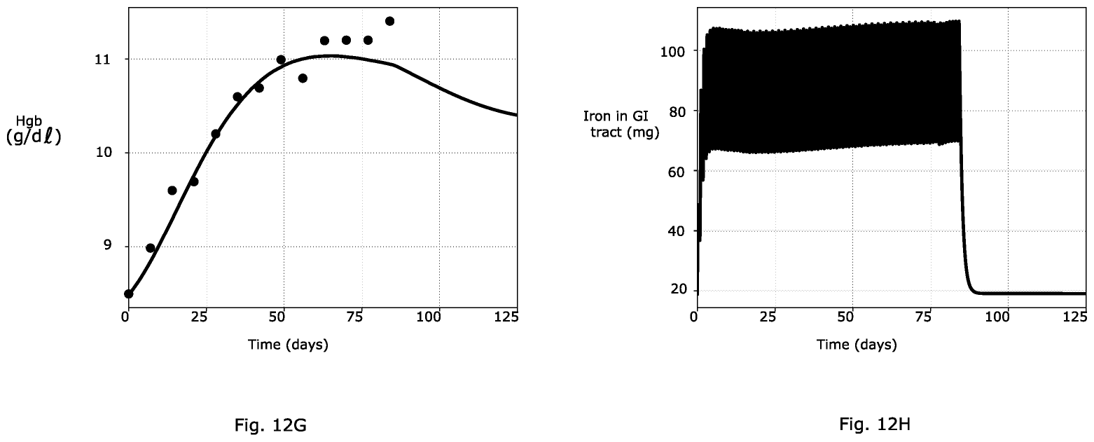

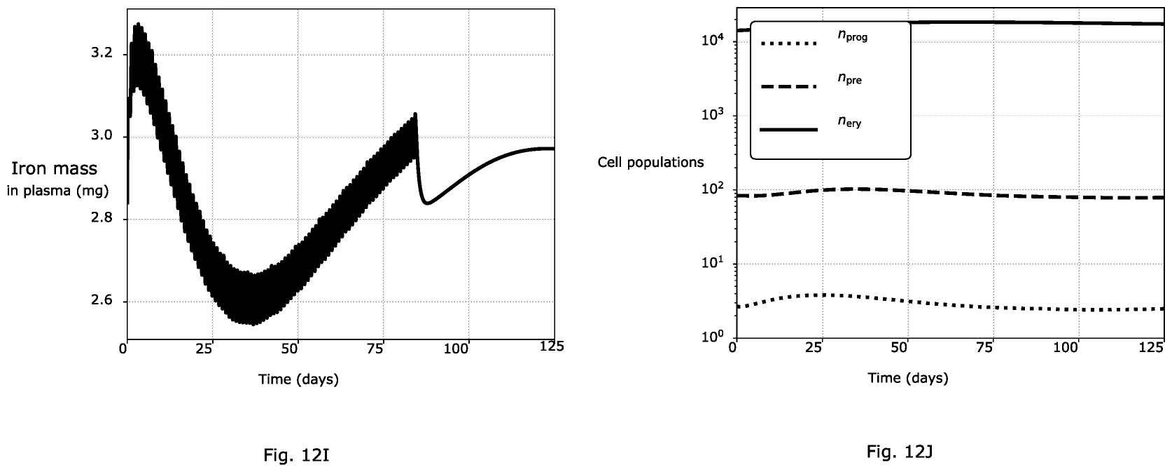

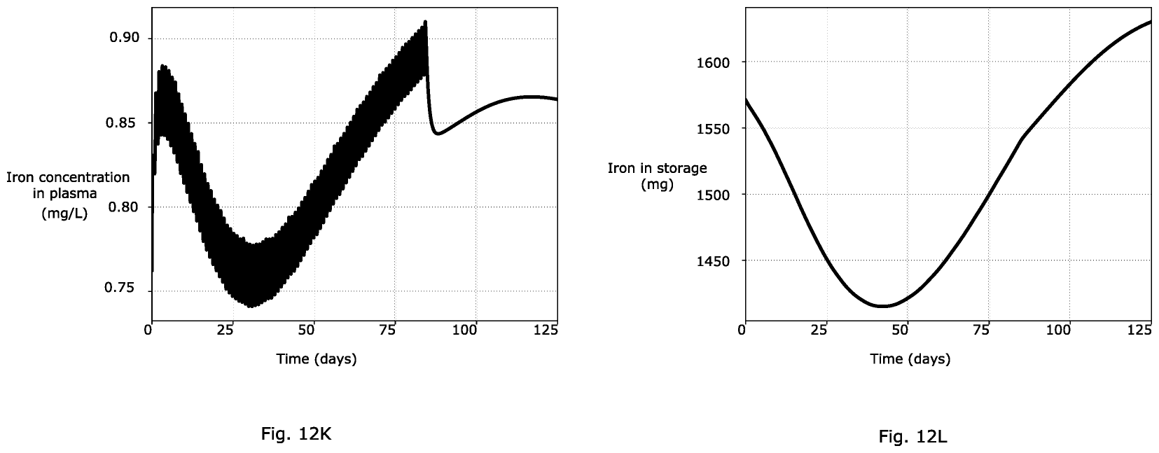

FIGS. 12A-12L show graphical representations for modeling HIF-PHI administrations according to one or more examples of the present application. The graphical representations of FIGS. 12A-12L show the output results (e.g., the personalized model parameters/variables described above) over a period of time, and for a specific set of patients (e.g., patients that took a series of HIF-PHI ROXADUSTAT administrations with concomitant oral iron administrations). For instance, FIG. 12A shows the HIF-PHI mass (model variables \tilde{s} and s from Eqs. 1.1 and 1.2-1.4, respectively) over a period of time. FIG. 12B shows the HIF signal (model variable h, Eq. 1.6) over a period of time. FIG. 12C shows the EPO mass (model variable e, Eq. 1.8) over a period of time. FIG. 12D shows the HIF-PHI serum concentration (model variable s, Eq. 1.1, divided by the plasma volume) over a period of time. FIG. 12E shows the Hepcidin (model variable p, Eq. 1.26) over a period of time. FIG. 12F shows the Ferritin (model variable

$${c}_{fer} = \frac{{ι}_{ms}}{{ι}_{ms}^{*} / {c}_{fer}^{*}} ,$$

Eq. 1.32) over a period of time. FIG. 12G shows the hemoglobin (model variable c_{hgb}, Eq. 1.7) over a period of time as well as shows the actual measured HGB data points (e.g., the dots). The data points digitized from FIG. 2 in Besarab et al., “Roxadustat (FG-4592): Correction of Anemia in Incident Dialysis Patients”, J. Am. Soc. Nephrol. 27, 1225-1233 (2016). In other words, FIG. 12G shows a curve denoting the calculated HGB based on inputting the patient data into the mathematical model as well as data points denoting the actual measured HGB from the patient data. FIG. 12H shows the iron mass in GI tract (model variable \tilde{ι}, Eq. 1.30) over a period of time. FIG. 12I shows the iron mass in plasma (model variable ιₚₗ, Eq. 1.32) over a period of time. FIG. 12J shows the abundance of cell populations (model variables n_{prog}, nₚᵣₑ, n_{ery}, Eqs. 1.9-1.11) over a period of time. FIG. 12K shows the iron concentration in plasma (model variable ιₚₗ, Eq. 1.32, divided by the plasma volume) over a period of time. FIG. 12L shows the iron mass in storage (model variable ιₘₛ, Eq. 1.32) over a period of time.

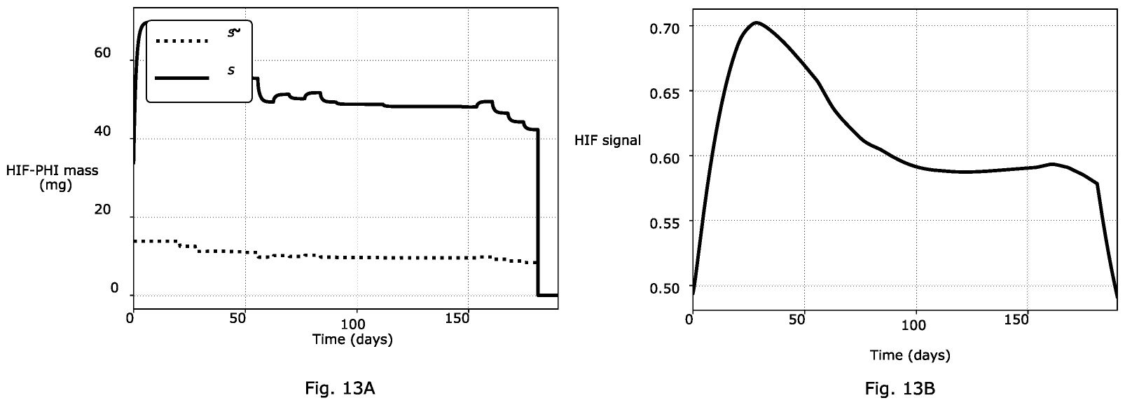

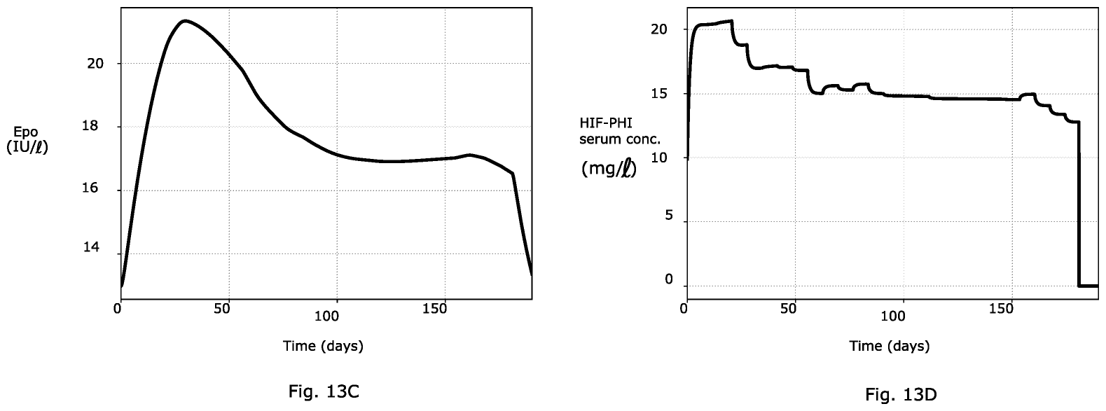

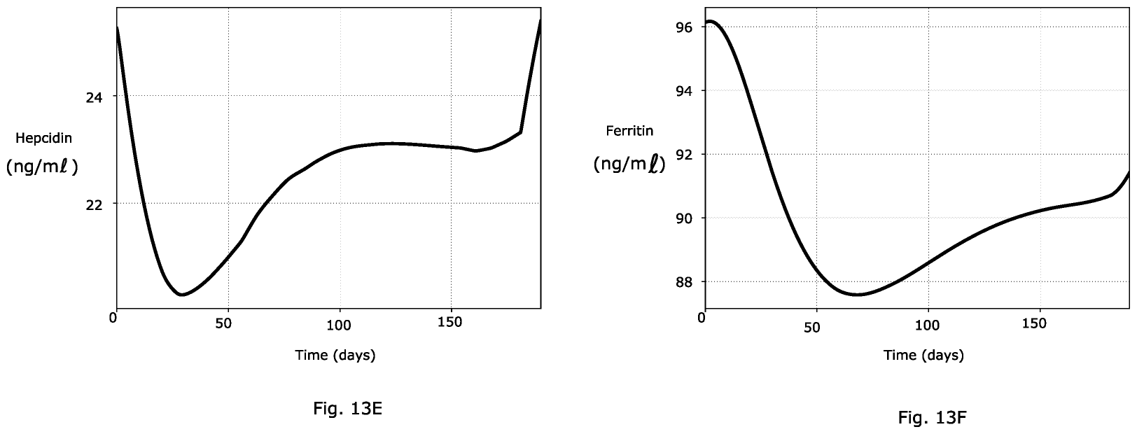

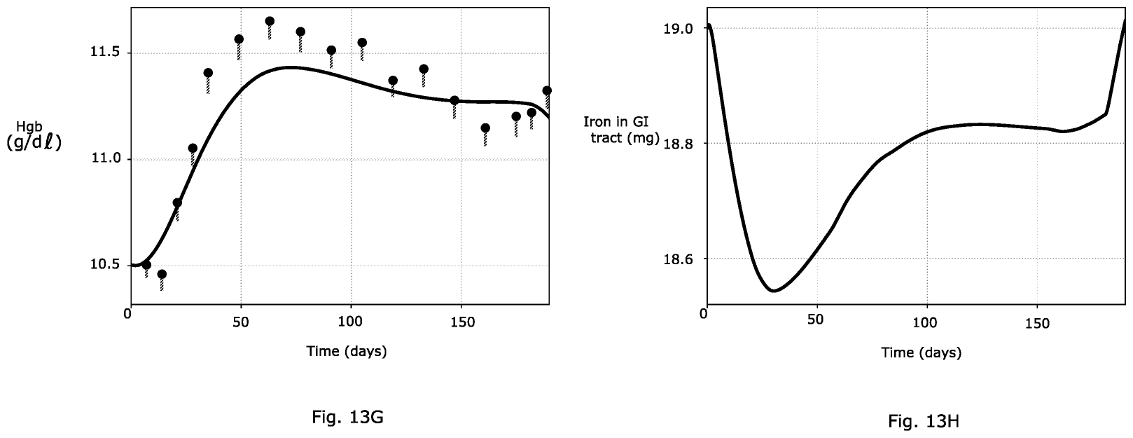

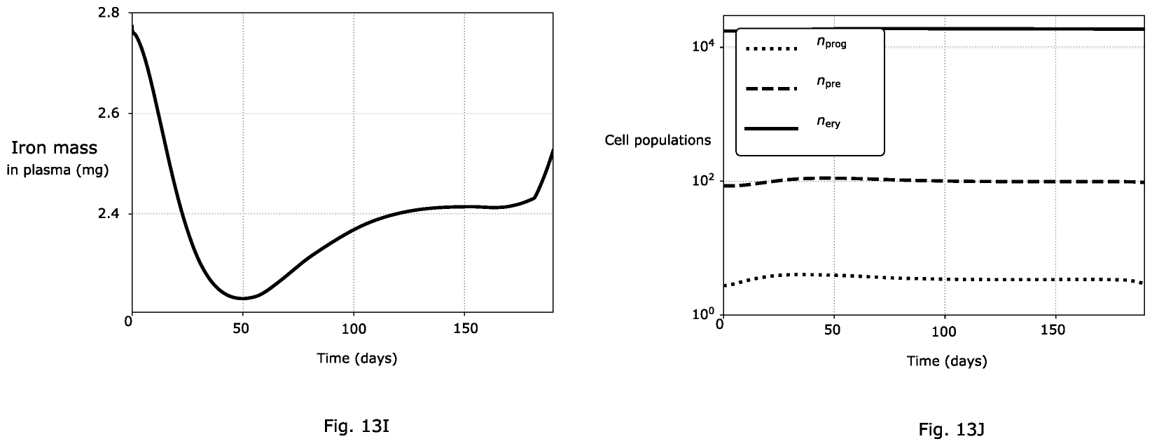

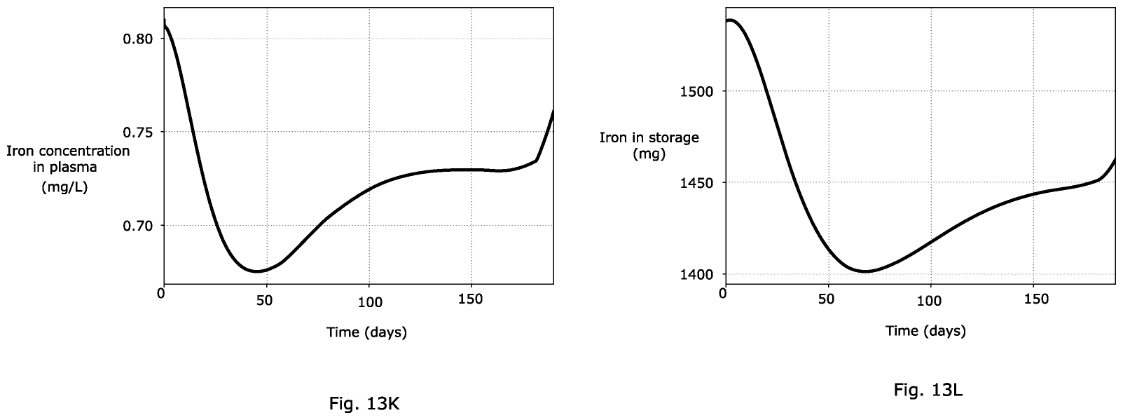

FIGS. 13A-13L show graphical representations for modeling HIF-PHI administrations according to one or more examples of the present application. The graphical representations of FIGS. 13A-13L show the output results (e.g., the personalized model parameters/variables described above) over a period of time, and for a specific set of patients (e.g., patients that took a series of HIF-PHI ROXADUSTAT administrations, and with a reference hepcidin production rate βₚ(θ) (Eq. 1.32), which represent patient populations with a C-reactive protein (CRP) concentration that is greater than an upper limit of the normal range (ULN)). For instance, FIG. 13A shows the HIF-PHI mass (model variables \tilde{s} and s from Eqs. 1.1 and 1.2-1.4, respectively) over a period of time. FIG. 13B shows the HIF signal (model variable h, Eq. 1.6) over a period of time. FIG. 13C shows the EPO mass (model variable e, Eq. 1.8) over a period of time. FIG. 13D shows the HIF-PHI serum concentration (model variable s, Eq. 1.1, divided by the plasma volume) over a period of time. FIG. 13E shows the Hepcidin (model variable p, Eq. 1.26) over a period of time. FIG. 13F shows the Ferritin (model variable

$${c}_{fer} = \frac{{ι}_{ms}}{{ι}_{ms}^{*} / {c}_{fer}^{*}} ,$$

Eq. 1.32) over a period of time. FIG. 13G shows the hemoglobin (model variable c_{hgb}, Eq. 1.7) over a period of time as well as shows the actual measured HGB data points (e.g., the dots). The data points are digitized from FIG. 2A in Chen et al., “Roxadustat Treatment for Anemia in Patients Undergoing Long-Term Dialysis”, New Engl. J. Med. 381, 1011-1022 (2019). In other words, FIG. 13G shows a curve denoting the calculated HGB based on inputting the patient data into the mathematical model as well as data points denoting the actual measured HGB from the patient data. FIG. 13H shows the iron mass in GI tract (model variable \tilde{ι}, Eq. 1.30) over a period of time. FIG. 13I shows the iron mass in plasma (model variable ιₚₗ, Eq. 1.32) over a period of time. FIG. 13J shows the abundance of cell populations (model variables n_{prog}, nₚᵣₑ, n_{ery}, Eqs. 1.9-1.11) over a period of time. FIG. 13K shows the iron concentration in plasma (model variable ιₚₗ, Eq. 1.32, divided by the plasma volume) over a period of time. FIG. 13L shows the iron mass in storage (model variable ιₘₛ, Eq. 1.32) over a period of time.

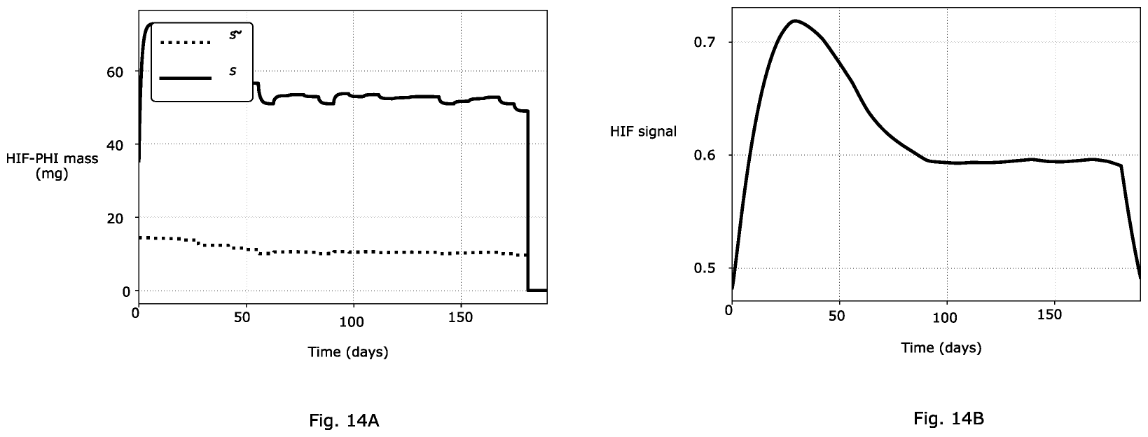

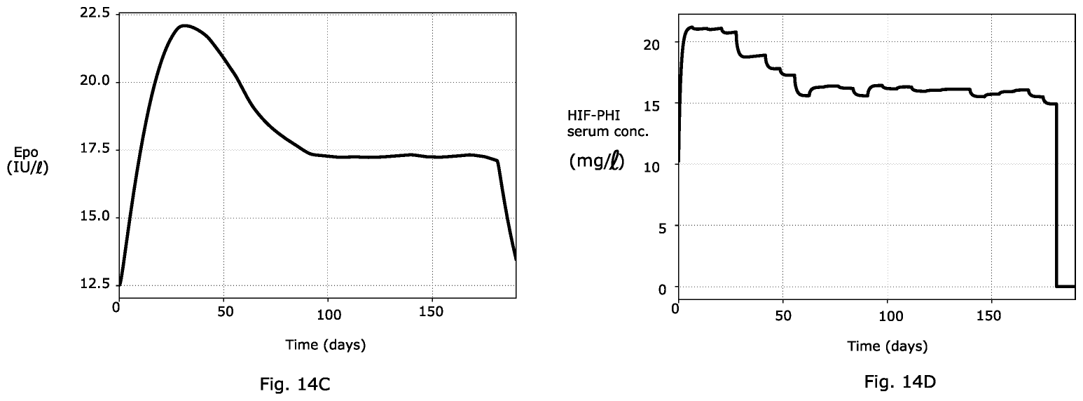

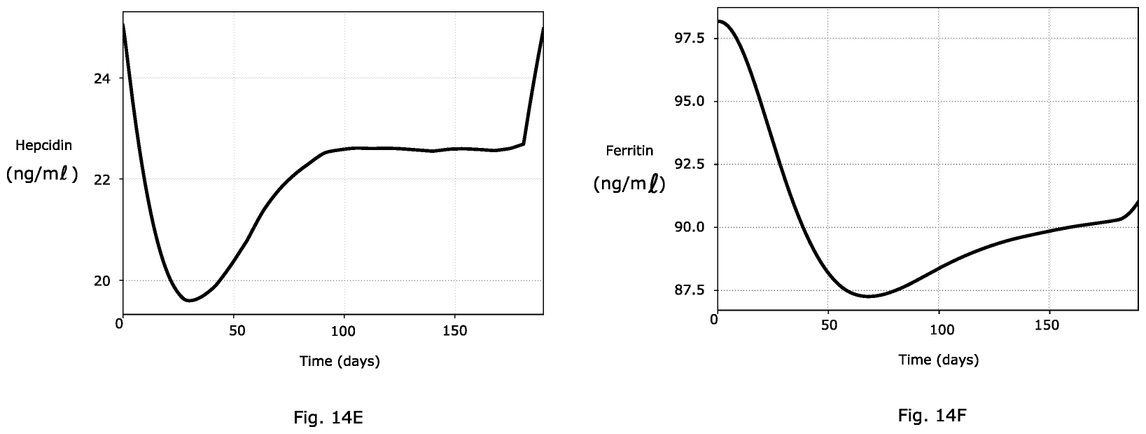

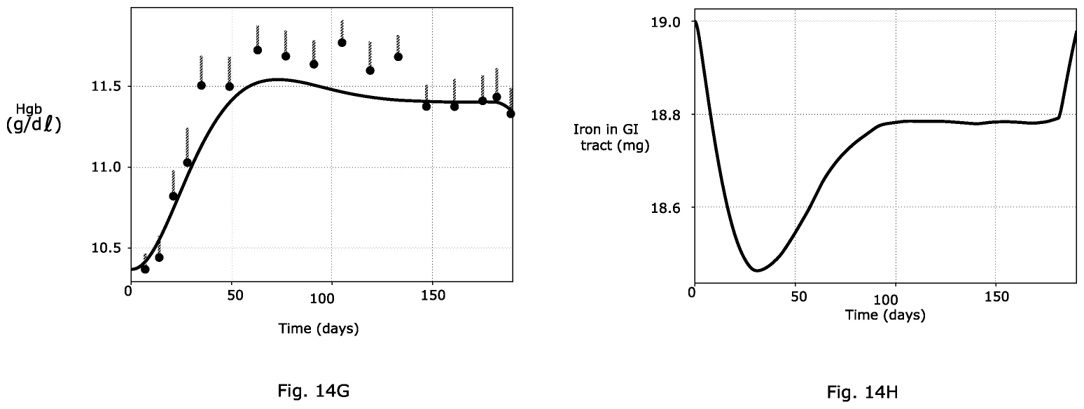

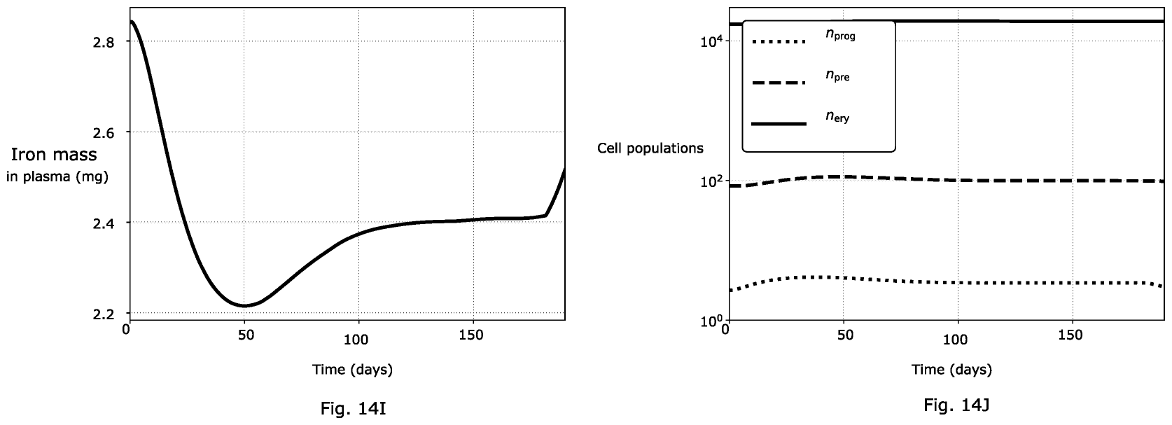

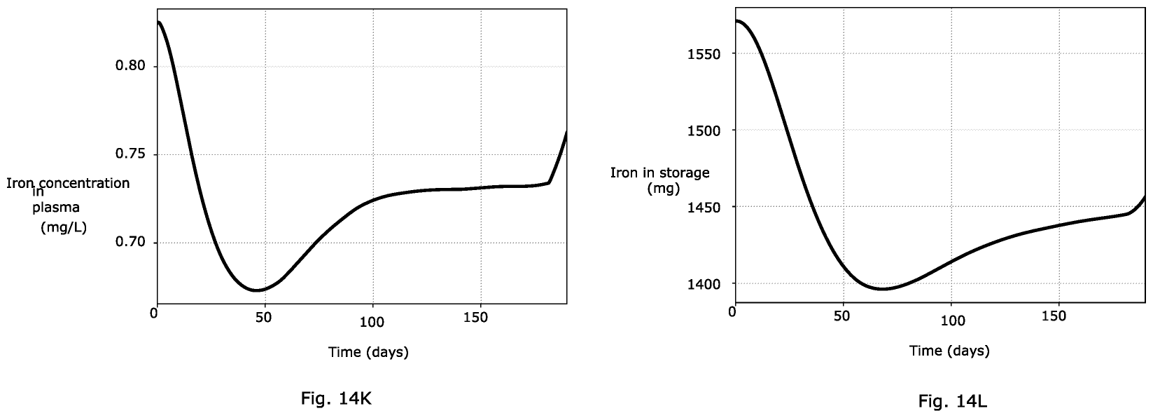

FIGS. 14A-14L show graphical representations for modeling HIF-PHI administrations according to one or more examples of the present application. The graphical representations of FIGS. 14A-14L show the output results (e.g., the personalized model parameters/variables described above) over a period of time, and for a specific set of patients (e.g., patients that took a series of HIF-PHI ROXADUSTAT administrations, and with a 2% increased hepcidin production rate βₚ(θ) (Eq. 1.32) as compared to a reference rate used in FIG. 13, which represent patient populations with a C-reactive protein (CRP) concentration that is less than an upper limit of the normal range (ULN)). For instance, FIG. 14A shows the HIF-PHI mass (model variables \tilde{s} and s from Eqs. 1.1 and 1.2-1.4, respectively) over a period of time. FIG. 14B shows the HIF signal (model variable h, Eq. 1.6) over a period of time. FIG. 14C shows the EPO mass (model variable e, Eq. 1.8) over a period of time. FIG. 14D shows the HIF-PHI serum concentration (model variable s, Eq. 1.1, divided by the plasma volume) over a period of time. FIG. 14E shows the Hepcidin (model variable p, Eq. 1.26) over a period of time. FIG. 14F shows the Ferritin (model variable

$${c}_{fer} = \frac{{ι}_{ms}}{{ι}_{ms}^{*} / {c}_{fer}^{*}} ,$$

Eq. 1.32) over a period of time. FIG. 14G shows the hemoglobin (model variable c_{hgb}, Eq. 1.7) over a period of time as well as shows the actual measured HGB data points (e.g., the dots). The data points are digitized from FIG. 2A in Chen et al., “Roxadustat Treatment for Anemia in Patients Undergoing Long-Term Dialysis”, New Engl. J. Med. 381, 1011-1022 (2019). In other words, FIG. 14G shows a curve denoting the calculated HGB based on inputting the patient data into the mathematical model as well as data points denoting the actual measured HGB from the patient data. FIG. 14H shows the iron mass in GI tract (model variable \tilde{ι}, Eq. 1.30) over a period of time. FIG. 14I shows the iron mass in plasma (model variable ιₚₗ, Eq. 1.32) over a period of time. FIG. 14J shows the abundance of cell populations (model variables n_{prog}, nₚᵣₑ, n_{ery}, Eqs. 1.9-1.11) over a period of time. FIG. 14K shows the iron concentration in plasma (model variable ιₚₗ, Eq. 1.32, divided by the plasma volume) over a period of time. FIG. 14L shows the iron mass in storage (model variable ιₘₛ, Eq. 1.32) over a period of time.

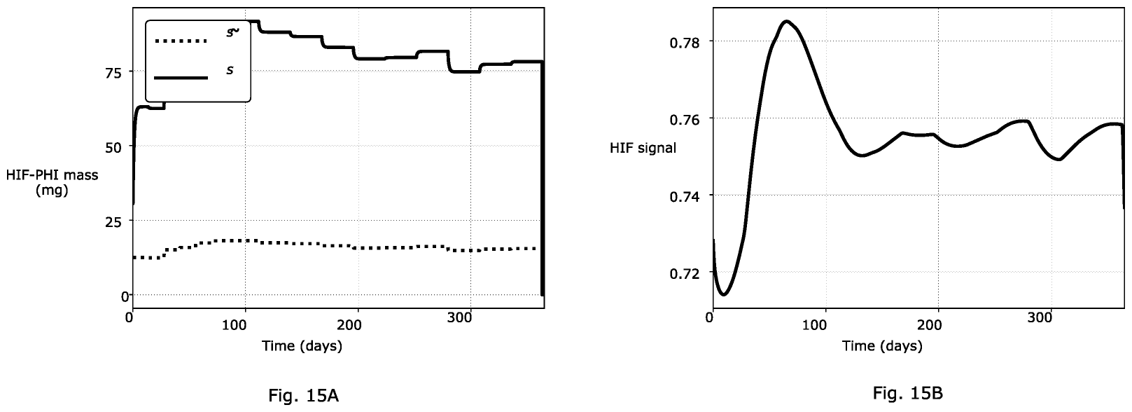

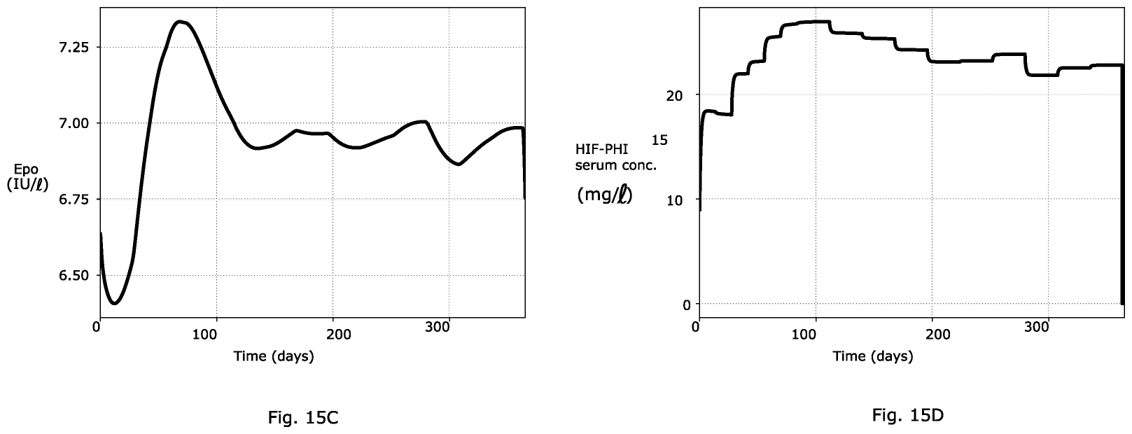

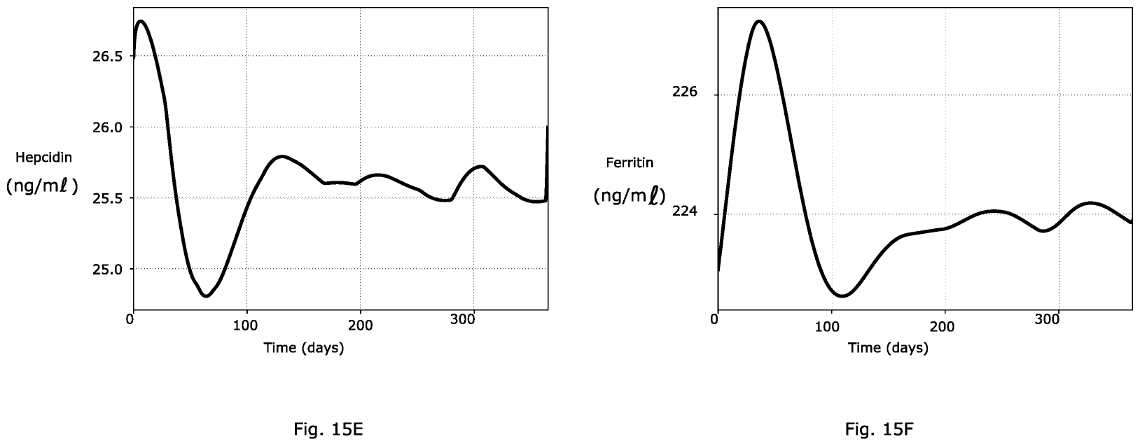

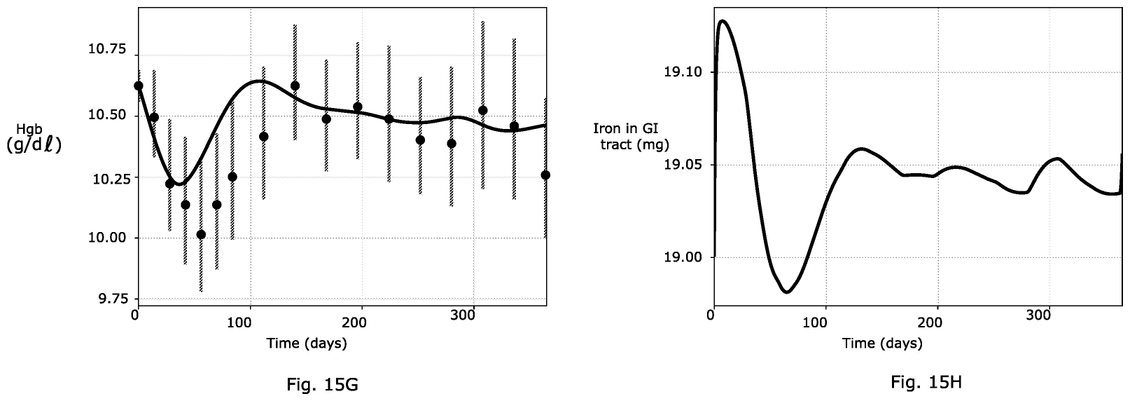

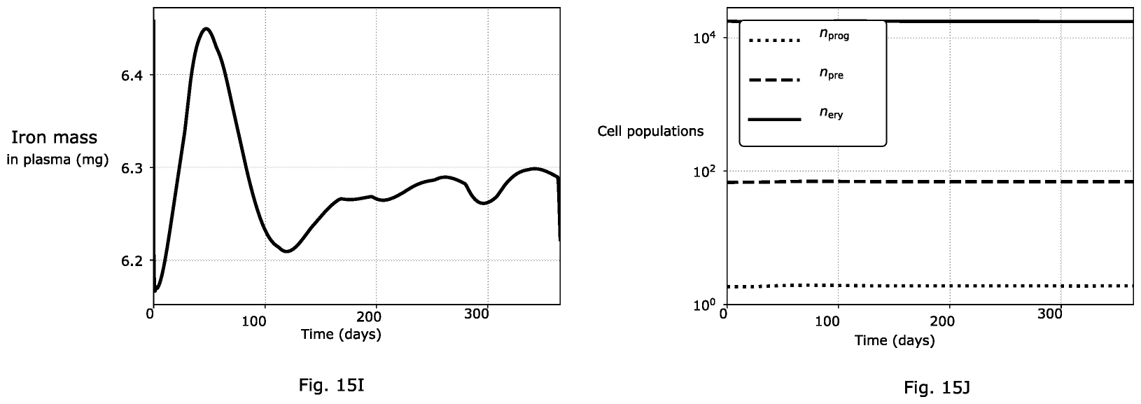

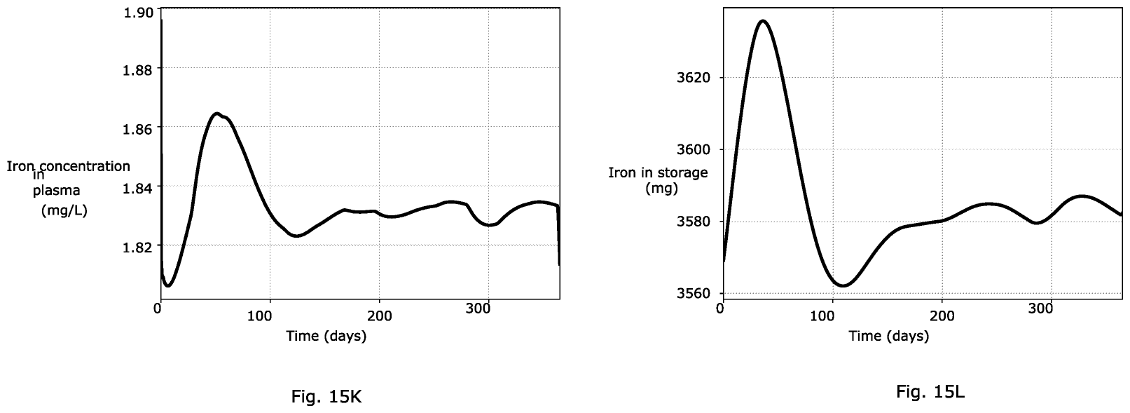

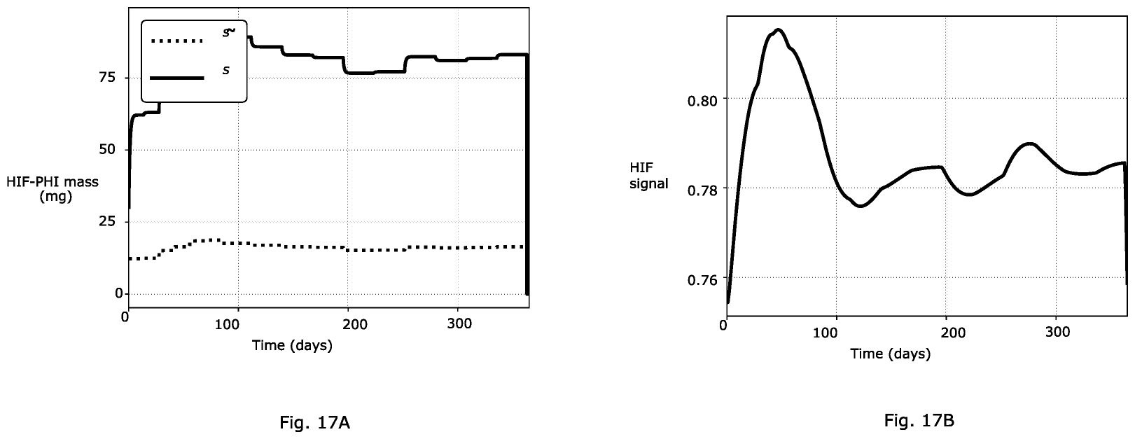

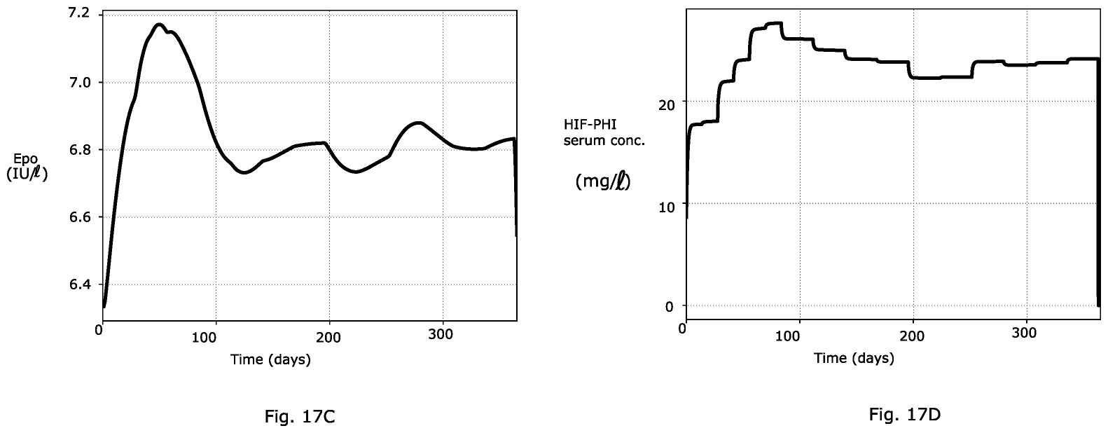

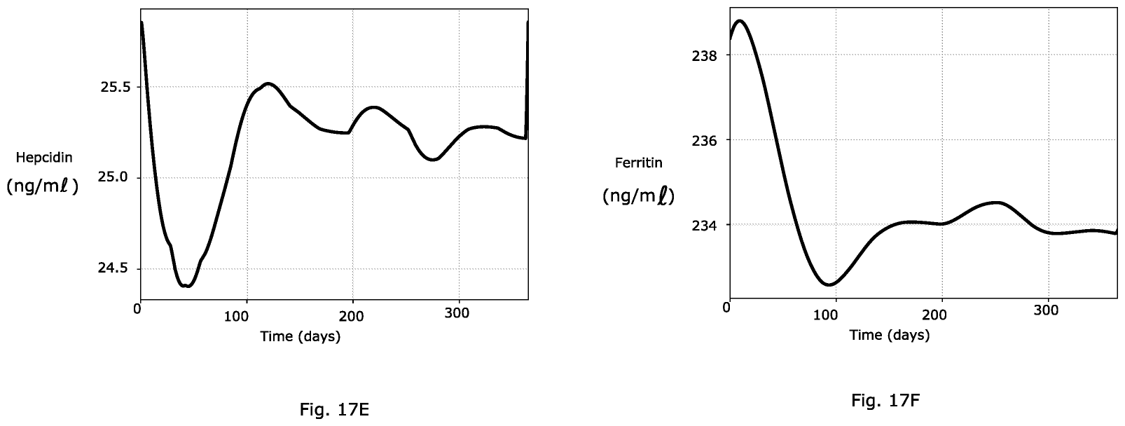

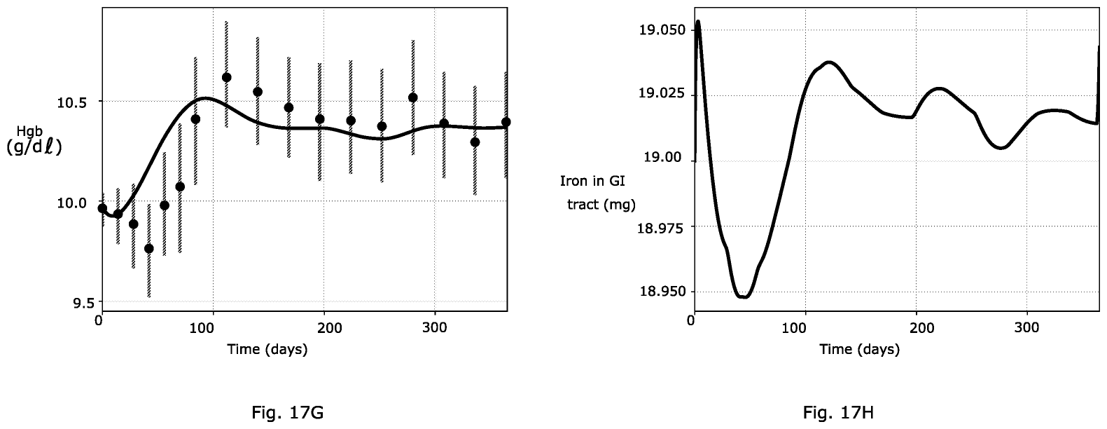

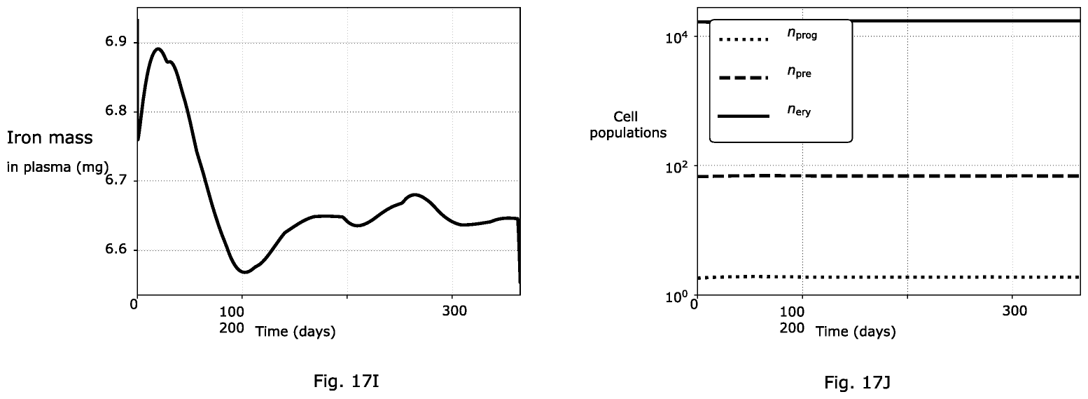

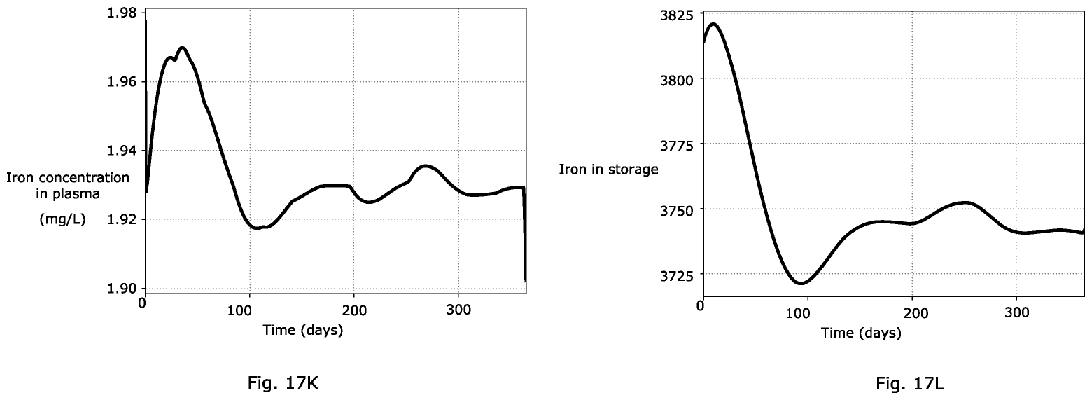

FIGS. 15A-15L show graphical representations for modeling HIF-PHI administrations according to one or more examples of the present application. The graphical representations of FIGS. 15A-15L show the output results (e.g., the personalized model parameters/variables described above) over a period of time, and for a specific set of patients (e.g., patients that took a series of HIF-PHI VADADUSTAT administrations, and with a 7% increased EPO production rate βₑ(θ) (Eq. 1.32) as compared to a reference rate used in FIG. 17, which represent patient populations with a base hemoglobin of between 10.4 g/dL and 11 g/dL). For instance, FIG. 15A shows the HIF-PHI mass (model variables \tilde{s} and s from Eqs. 1.1 and 1.2-1.4, respectively) over a period of time. FIG. 15B shows the HIF signal (model variable h, Eq. 1.6) over a period of time. FIG. 15C shows the EPO mass (model variable e, Eq. 1.8) over a period of time. FIG. 15D shows the HIF-PHI serum concentration (model variable s, Eq. 1.1, divided by the plasma volume) over a period of time. FIG. 15E shows the Hepcidin (model variable p, Eq. 1.26) over a period of time. FIG. 15F shows the Ferritin (model variable

$${c}_{fer} = \frac{{ι}_{ms}}{{ι}_{ms}^{*} / {c}_{fer}^{*}} ,$$

Eq. 1.32) over a period of time. FIG. 15G shows the hemoglobin (model variable c_{hgb}, Eq. 1.7) over a period of time as well as shows the actual measured HGB data points (e.g., the dots). The data points are digitized from FIG. 2A in Nangaku et al., “Efficacy and safety of vadadustat compared with darbepoetin alfa in Japanese anemic patients on hemodialysis: a Phase 3 multicenter, randomized, double-blind study”, Nephrol. Dial. Transplant. 36, 1731-1741 (2021). In other words, FIG. 15G shows a curve denoting the calculated HGB based on inputting the patient data into the mathematical model as well as data points denoting the actual measured HGB from the patient data. FIG. 15H shows the iron mass in GI tract (model variable \tilde{ι}, Eq. 1.30) over a period of time. FIG. 15I shows the iron mass in plasma (model variable ιₚₗ, Eq. 1.32) over a period of time. FIG. 15J shows the abundance of cell populations (model variables n_{prog}, nₚᵣₑ, n_{ery}, Eqs. 1.9-1.11) over a period of time. FIG. 15K shows the iron concentration in plasma (model variable ιₚₗ, Eq. 1.32, divided by the plasma volume) over a period of time. FIG. 15L shows the iron mass in storage (model variable ιₘₛ, Eq. 1.32) over a period of time.

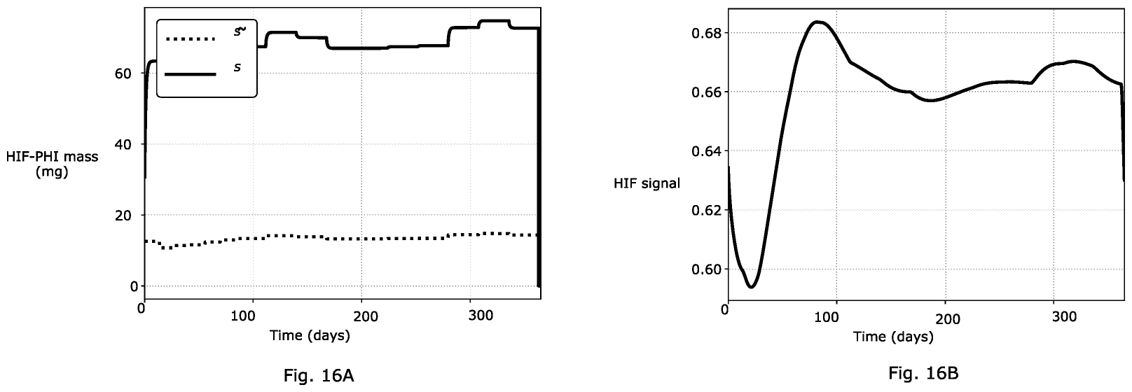

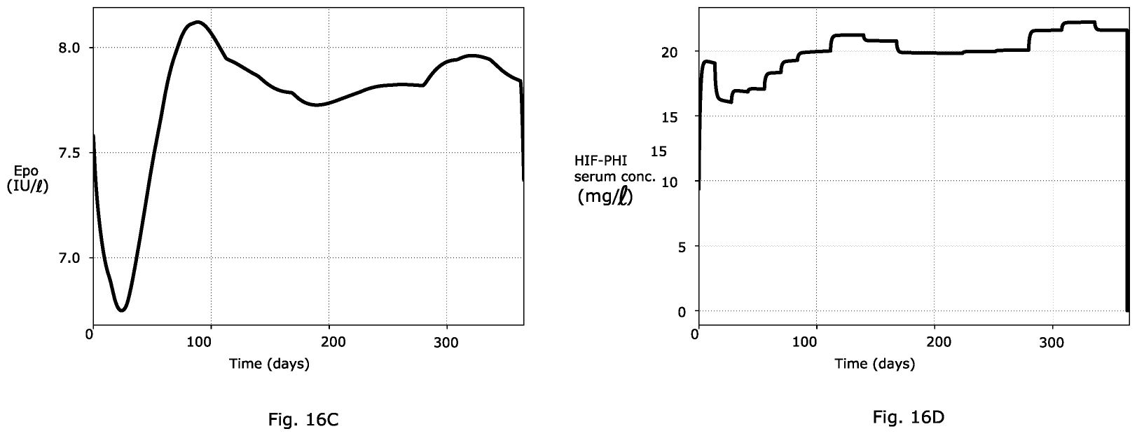

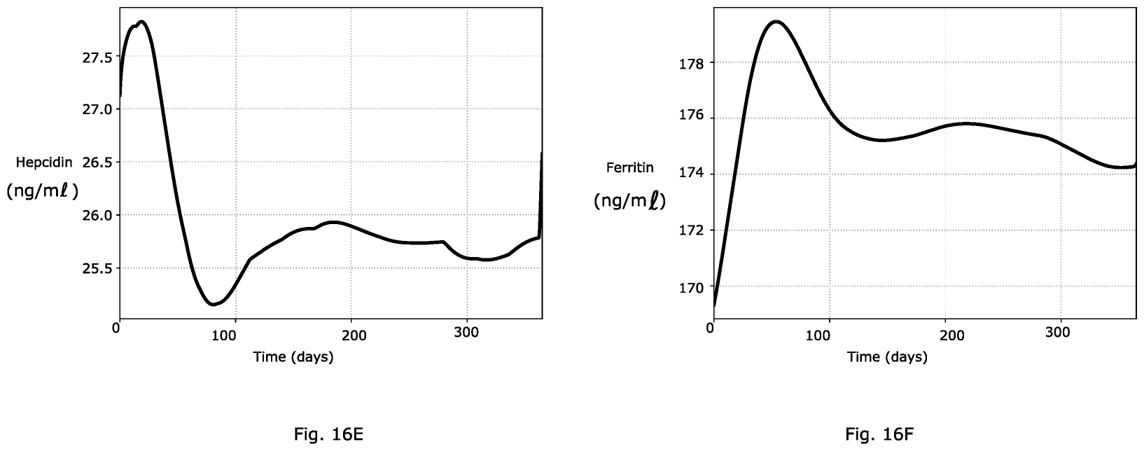

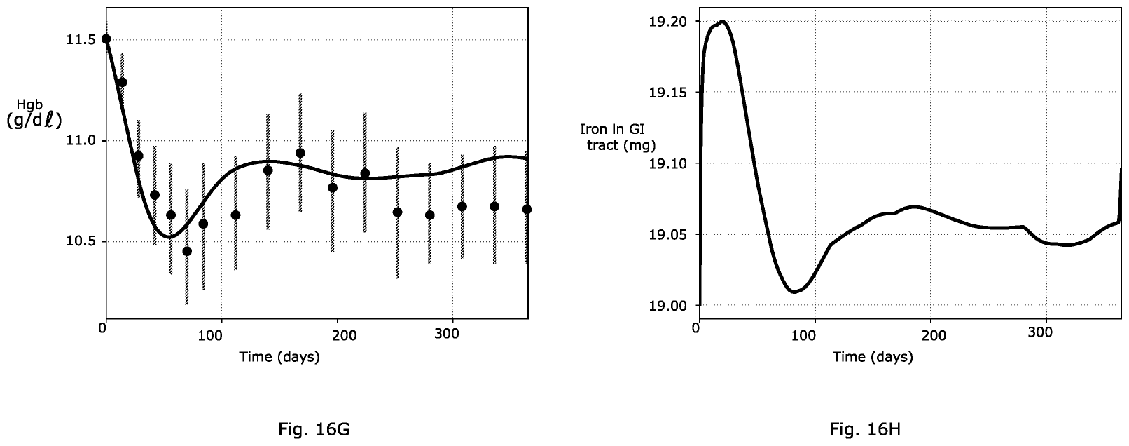

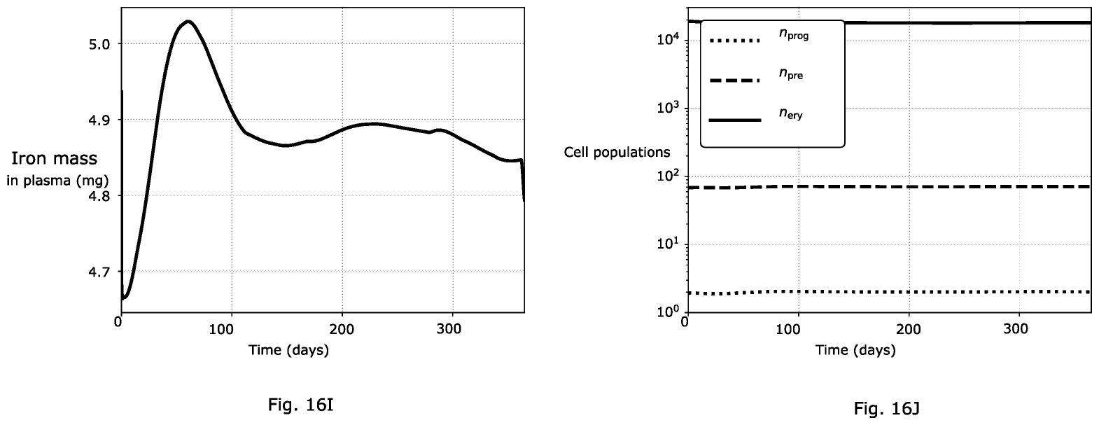

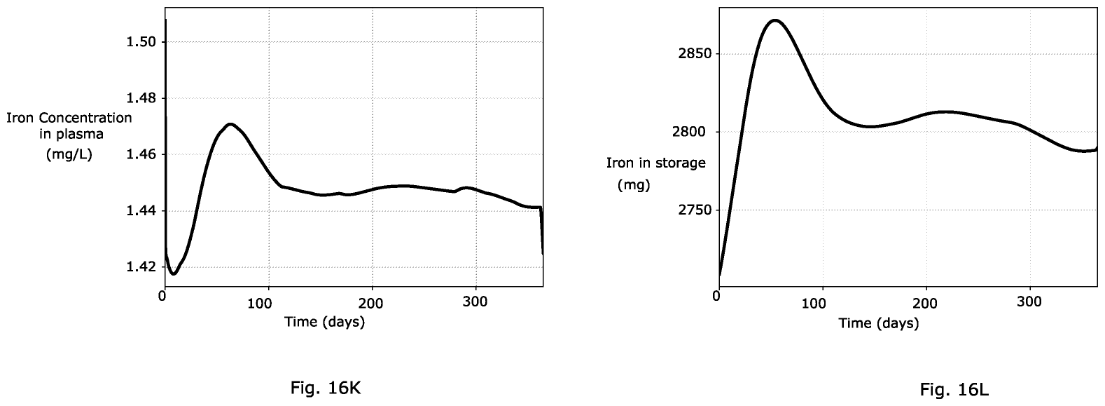

FIGS. 16A-16L show graphical representations for modeling HIF-PHI administrations according to one or more examples of the present application. The graphical representations of FIGS. 16A-16L show the output results (e.g., the personalized model parameters/variables described above) over a period of time, and for a specific set of patients (e.g., patients that took a series of HIF-PHI VADADUSTAT administrations, and with a 42% increased EPO production rate βₑ(θ) (Eq. 1.32) as compared to a reference rate used in FIG. 17, which represent patient populations with a base hemoglobin greater than 11 g/dL). For instance, FIG. 16A shows the HIF-PHI mass (model variables \tilde{s} and s from Eqs. 1.1 and 1.2-1.4, respectively) over a period of time. FIG. 16B shows the HIF signal (model variable h, Eq. 1.6) over a period of time. FIG. 16C shows the EPO mass (model variable e, Eq. 1.8) over a period of time. FIG. 16D shows the HIF-PHI serum concentration (model variable s, Eq. 1.1, divided by the plasma volume) over a period of time. FIG. 16E shows the Hepcidin (model variable p, Eq. 1.26) over a period of time. FIG. 16F shows the Ferritin (model variable

$${c}_{fer} = \frac{{ι}_{ms}}{{ι}_{ms}^{*} / {c}_{fer}^{*}} ,$$

Eq. 1.32) over a period of time. FIG. 16G shows the hemoglobin (model variable c_{hgb}, Eq. 1.7) over a period of time as well as shows the actual measured HGB data points (e.g., the dots). The data points are digitized from FIG. 2A in Nangaku et al., “Efficacy and safety of vadadustat compared with darbepoetin alfa in Japanese anemic patients on hemodialysis: a Phase 3 multicenter, randomized, double-blind study”, Nephrol. Dial. Transplant. 36, 1731-1741 (2021). In other words, FIG. 16G shows a curve denoting the calculated HGB based on inputting the patient data into the mathematical model as well as data points denoting the actual measured HGB from the patient data. FIG. 16H shows the iron mass in GI tract (model variable \tilde{ι}, Eq. 1.30) over a period of time. FIG. 16I shows the iron mass in plasma (model variable ιₚₗ, Eq. 1.32) over a period of time. FIG. 16J shows the abundance of cell populations (model variables n_{prog}, nₚᵣₑ, n_{ery}, Eqs. 1.9-1.11) over a period of time. FIG. 16K shows the iron concentration in plasma (model variable ιₚₗ, Eq. 1.32, divided by the plasma volume) over a period of time. FIG. 16L shows the iron mass in storage (model variable ιₘₛ, Eq. 1.32) over a period of time.

FIGS. 17A-17L show graphical representations for modeling HIF-PHI administrations according to one or more examples of the present application. The graphical representations of FIGS. 17A-17L show the output results (e.g., the personalized model parameters/variables described above) over a period of time, and for a specific set of patients (e.g., patients that took a series of HIF-PHI VADADUSTAT administrations, and with a reference EPO production rate βₑ(θ) (Eq. 1.32), which represent patient populations with a base hemoglobin less than 10.4 g/dL). For instance, FIG. 17A shows the HIF-PHI mass (model variables \tilde{s} and s from Eqs. 1.1 and 1.2-1.4, respectively) over a period of time. FIG. 17B shows the HIF signal (model variable h, Eq. 1.6) over a period of time. FIG. 17C shows the EPO mass (model variable e, Eq. 1.8) over a period of time. FIG. 17D shows the HIF-PHI serum concentration (model variable s, Eq. 1.1, divided by the plasma volume) over a period of time. FIG. 17E shows the Hepcidin (model variable p, Eq. 1.26) over a period of time. FIG. 17F shows the Ferritin (model variable

$${c}_{fer} = \frac{{ι}_{ms}}{{ι}_{ms}^{*} / {c}_{fer}^{*}} ,$$

Eq. 1.32) over a period of time. FIG. 17G shows the hemoglobin (model variable c_{hgb}, Eq. 1.7) over a period of time as well as shows the actual measured HGB data points (e.g., the dots). The data points are digitized from FIG. 2A in Nangaku et al., “Efficacy and safety of vadadustat compared with darbepoetin alfa in Japanese anemic patients on hemodialysis: a Phase 3 multicenter, randomized, double-blind study”, Nephrol. Dial. Transplant. 36, 1731-1741 (2021). In other words, FIG. 17G shows a curve denoting the calculated HGB based on inputting the patient data into the mathematical model as well as data points denoting the actual measured HGB from the patient data. FIG. 17H shows the iron mass in GI tract (model variable \tilde{ι}, Eq. 1.30) over a period of time. FIG. 17I shows the iron mass in plasma (model variable ιₚₗ, Eq. 1.32) over a period of time. FIG. 17J shows the abundance of cell populations (model variables n_{prog}, nₚᵣₑ, n_{ery}, Eqs. 1.9-1.11) over a period of time. FIG. 17K shows the iron concentration in plasma (model variable ιₚₗ, Eq. 1.32, divided by the plasma volume) over a period of time. FIG. 17L shows the iron mass in storage (model variable ιₘₛ, Eq. 1.32) over a period of time.

It will be appreciated that the various machine-implemented operations described herein may occur via the execution, by one or more respective processors, of processor-executable instructions stored on a tangible, non-transitory computer-readable medium, such as a random access memory (RAM), read-only memory (ROM), programmable read-only memory (PROM), and/or another electronic memory mechanism. Thus, for example, operations performed by any device described herein may be carried out according to instructions stored on and/or applications installed on the device, and via software and/or hardware of the device.

All references, including publications, patent applications, and patents, cited herein are hereby incorporated by reference to the same extent as if each reference were individually and specifically indicated to be incorporated by reference and were set forth in its entirety herein.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present application covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method of determining a next hypoxia-inducible factor prolyl hydroxylase inhibitor (HIF-PHI) dosage for a first patient using a patient HIF-PHI model, comprising:
obtaining population patient data associated with a plurality of patients, wherein the population patient data indicates previous HIF-PHI dosages and hemoglobin measurements for the plurality of patients;
generating a plurality of virtual patient avatars based on the population patient data, wherein each of the plurality of virtual patient avatars indicates a set of personalized model parameters;
determining a plurality of HIF-PHI models for the plurality of virtual patient avatars based on the set of personalized model parameters;
determining one or more HIF-PHI treatment schemes for administration of HIF-PHI dosages based on the plurality of HIF-PHI models; and
administering the next HIF-PHI dosage for the first patient, of the plurality of patients, based on using the one or more determined HIF-PHI treatment schemes and a hematocrit and/or hemoglobin concentration for the first patient.

2. The method of claim 1, further comprising:
obtaining individualized patient data for a second patient, of the plurality of patients, wherein the individualized patient data indicates a previous HIF-PHI dosage and a hemoglobin measurement for the second patient;
determining a patient HIF-PHI model for the second patient based on the previous HIF-PHI dosage, the hemoglobin measurement, and a mathematical model for hydroxylase inhibitor (HIF) stabilizer treatment, wherein the HIF-PHI model indicates a set of individualized model parameters for the second patient;
based on the second patient's hemoglobin measurement being outside of a patient threshold, employing the patient HIF-PHI model to determine a next HIF-PHI dosage for the second patient; and
administering the next HIF-PHI dosage to the second patient to adjust the hemoglobin concentration to be within the patient threshold.

3. The method of claim 2, wherein administering the next HIF-PHI dosage comprises:
causing display of the next HIF-PHI dosage on a display device.

4. The method of claim 1, further comprising:
obtaining a mathematical model for hydroxylase inhibitor (HIF) stabilizer treatment;
wherein determining the plurality of HIF-PHI models for the plurality of virtual patient avatars comprises generating the plurality of HIF-PHI models by inserting the set of personalized model parameters into the mathematical model.

5. The method of claim 4, wherein each of the one or more HIF-PHI treatment schemes indicates a decision tree comprising a plurality of branches indicating different HIF-PHI dosages based on hemoglobin concentrations.

6. The method of claim 5, wherein determining the one or more HIF-PHI treatment schemes comprises:
generating a plurality of HIF-PHI treatment schemes for performing HIF stabilizer treatment;
simulating a plurality of virtual trials using the plurality of HIF-PHI treatment schemes and the plurality of HIF-PHI models for the plurality of virtual patient avatars; and
determining the one or more HIF-PHI treatment schemes based on simulating the plurality of virtual trials.

7. The method of claim 6, wherein determining the one or more HIF-PHI treatment schemes is based on a number of patients, from the plurality of patients, within a target hemoglobin threshold and an amount of HIF-PHI medication administered to a plurality of simulated patients within the plurality of virtual trials.

8. The method of claim 6, wherein administering the next HIF-PHI dosage for the first patient comprises causing display of the next HIF-PHI dosage for the first patient on a display device.

9. The method of claim 1, wherein the set of personalized model parameters comprises a HIF-PHI bioavailability parameter, a red blood cell (RBC) lifespan parameter, and a hemoglobin set point parameter, and
wherein generating the plurality of virtual patient avatars comprises determining the HIF-PHI bioavailability parameter, the red blood cell (RBC) lifespan parameter, and the hemoglobin set point parameter for each of the plurality of virtual patient avatars.

10. The method of claim 9, wherein the set of personalized model parameters further comprises a basal erythropoietin (EPO) synthesis rate parameter, a HIF signal threshold parameter, and a hepcidin decay rate parameter, and
wherein generating the plurality of virtual patient avatars further comprises determining the basal EPO synthesis rate parameter, the HIF signal threshold parameter, and the hepcidin decay rate parameter for each of the plurality of virtual patient avatars.

11. The method of claim 1, wherein determining the plurality of HIF-PHI models for the plurality of virtual patient avatars is further based on the following equation:
$$\frac{dh}{dt} = {β}_{h} ( θ ) [ {φ}_{{a}_{h} , {p}_{h} , {q}_{h , 0}} ( {k}_{h} [ {c}_{hgb} - {c}_{hgb}^{*} ] ) + {E}_{s} {ϕ}^{+} ( {[ \frac{s}{\overline{s}} ]}^{{m}_{h}} ) ] - {k}_{h} h ,$$
where dh/dt indicates a rate of change of HIF signaling activity, βₕ(θ) indicates a formation rate of HIF complexes, φₐ_{ₕ}_{,b}_{ₕ}_{,q}_{_{h,0}} indicates a hypoxic upregulation of a HIF signal in response to decreases in blood hemoglobin concentration, kₕ indicates a decay rate of the HIF signal, c_{hgb} indicates a blood hemoglobin concentration measured by kidneys, c*_{hgb} indicates a physiological hemoglobin set point, Eₛ indicates a HIF-PHI responsiveness,
$${ϕ}^{+} ( {[ \frac{s}{\overline{s}} ]}^{{m}_{h}} )$$
indicates upregulation of the HIF signal in response to HIF-PHI administrations, and h indicates the HIF signal.

12. The method of claim 1, wherein determining the plurality of HIF-PHI models for the plurality of virtual patient avatars is further based on the following equation:
$$\frac{de}{dt} = {β}_{e} ( θ ) + {β}_{e} ( θ ) {φ}_{{a}_{e} , {b}_{e} , {q}_{e , 0}} ( {k}_{e} h ) - {k}_{e} e ,$$
where de/dt indicates a EPO concentration rate of change, βₑ(θ) indicates basal erythropoietin (EPO) synthesis under steady-state conditions, βₑ(θ)φₐ_{ₑ}_{,b}_{ₑ}_{,q}_{_{e,0}}(kₑh) indicates additional EPO synthesis due to activation by a HIF signal, and kₑe indicates EPO decay with a decay rate kₑ.

13. The method of claim 1, wherein determining the plurality of HIF-PHI models for the plurality of virtual patient avatars is further based on the following equation:
$$\frac{{dn}_{ery}}{dt} = {2}^{{d}_{pre}} {J}_{pre \to ery} - {K}_{ery} - {L}_{bleeding} + {L}_{donation} ,$$
where dn_{ery}/dt indicates a rate of change of the total amount of red blood cells, 2^{d}^{ₚᵣₑ} J_{pre→ery} indicates a differentiation flux from a precursors population to an erythrocytes population, K_{ery} indicates cell fluxes due to apoptosis, L_{bleeding} indicates cell fluxes due to bleeding events, and L_{donation} indicates cell fluxes due to blood donation events.

14. A method of adjusting a patient's hematocrit and/or hemoglobin concentration using a patient hypoxia-inducible factor prolyl hydroxylase inhibitor (HIF-PHI) model, comprising:
obtaining individualized patient data for the patient, wherein the individualized patient data indicates a previous HIF-PHI dosage and a hemoglobin measurement for the patient;
determining a patient HIF-PHI model for the patient based on the previous HIF-PHI dosage, the hemoglobin measurement, and a mathematical model for hydroxylase inhibitor (HIF) stabilizer treatment, wherein the HIF-PHI model indicates a set of individualized model parameters for the patient;
based on the patient's hematocrit and/or hemoglobin concentration being outside of a patient threshold, employing the patient HIF-PHI model to determine a next HIF-PHI dosage for the patient; and
administering the next HIF-PHI dosage to the patient to adjust the hematocrit and/or the hemoglobin concentration to be within the patient threshold.

15. The method of claim 14, wherein administering the next HIF-PHI dosage comprises:
causing display of the next HIF-PHI dosage on a display device.

16. The method of claim 14, wherein the set of individualized model parameters comprises a HIF-PHI bioavailability parameter, a red blood cell (RBC) lifespan parameter, and a hemoglobin set point parameter, and
wherein determining the next HIF-PHI dosage for the patient is based on using the HIF-PHI bioavailability parameter, the red blood cell (RBC) lifespan parameter, and the hemoglobin set point parameter.

17. The method of claim 16, wherein the set of individualized model parameters further comprises a basal erythropoietin (EPO) synthesis rate parameter, a HIF signal threshold parameter, and a hepcidin decay rate parameter, and
wherein determining the next HIF-PHI dosage for the patient is further based on the basal EPO synthesis rate parameter, the HIF signal threshold parameter, and the hepcidin decay rate parameter.

18. The method of claim 14, wherein employing the patient HIF-PHI model to determine the next HIF-PHI dosage for the patient comprises:
inputting a plurality of next HIF-PHI dosages into the HIF-PHI model to determine a plurality of outputs indicating expected hematocrit or hemoglobin concentrations; and
selecting the next HIF-PHI dosage from the plurality of next HIF-PHI dosages based on an output, of the plurality of outputs, being within the patient threshold.

19. The method of claim 14, wherein employing the patient HIF-PHI model to determine the next HIF-PHI dosage for the patient comprises:
inputting a plurality of next HIF-PHI dosages into the HIF-PHI model to determine a plurality of outputs indicating expected hematocrit or hemoglobin concentrations;
determining a subset of next HIF-PHI dosages, of the plurality of next HIF-PHI dosages, based on one or more outputs, of the plurality of outputs, associated with the subset of next HIF-PHI dosages being within the patient threshold; and
selecting the next HIF-PHI dosage from the subset of next HIF-PHI dosages based on the next HIF-PHI dosage being a lowest amount of HIF-PHI dosage within the subset of next HIF-PHI dosages.

20. A computing device, comprising:
a display configured to display information associated with a patient hypoxia-inducible factor prolyl hydroxylase inhibitor (HIF-PHI) model;
one or more processors; and
a non-transitory computer-readable medium having processor-executable instructions stored thereon, wherein the processor-executable instructions, when executed by the one or more processors, facilitate:
obtaining population patient data associated with a plurality of patients, wherein the population patient data indicates previous HIF-PHI dosages and hemoglobin measurements for the plurality of patients;
generating a plurality of virtual patient avatars based on the population patient data, wherein each of the plurality of virtual patient avatars indicates a set of personalized model parameters;
determining a plurality of HIF-PHI models for the plurality of virtual patient avatars based on the set of personalized model parameters;
determining one or more HIF-PHI treatment schemes for administration of HIF-PHI dosages based on the plurality of HIF-PHI models; and
displaying the next HIF-PHI dosage for the first patient, of the plurality of patients, on the display based on using the one or more determined HIF-PHI treatment schemes and a hematocrit and/or hemoglobin concentration for the first patient.
